SEQUENCE LISTING (39 sequences)

SEQ ID NO: 1 - DNA, 5546 bp, Drosophila
  Drosophila Serrate (Ser)
ccgagtcgagcgccgtgcttcgagcggtgatgagccccttttctgtcaacgctaaagatctacaaaacatcagcgcctatcaagtggaagtgtcaagtgtgaacaaaacaaaaacgagagaagcacatactaaggtccatataaataataaataataattgtgtgtgataacaacattatccaaacaaaaccaaacaaaacgaaggcaaagtggagaaaatgatacagcatccagagtacggccagttattcagctatccagagcaagtgtagtgtggcaaaatagaaacaaacaaaggcaccaaaatctgcatacatgggctaattaaggctgcccagcgaatttacatttgtgtggtgccaatccagagtgaatccgaaacaaactccatctagatcgccaaccagcatcacgctcgcaaacgcccccagaatgtacaaaatgtttaggaaacattttcggcgaaaaccagctacgtcgtcgtcgttggagtcaacaatagaatcagcagacagcctgggaatgtccaagaagacggcgacaaaaaggcagcgtccgaggcatcgggtacccaaaatcgcgaccctgccatcgacgatccgcgattgtcgatcattaaagtctgcctgcaacttaattgctttaattttaatactgttagtccataagatatccgcagctggtaacttcgagctggaaatattagaaatctcaaataccaacagccatctactcaacggctattgctgcggcatgccagcggaacttagggccaccaagacgataggctgctcgccatgcacgacggcattccggctgtgcctgaaggagtaccagaccacggagcagggtgccagcatatccacgggctgttcgtttggcaacgccaccaccaagatactgggtggctccagctttgtgctcagcgatccgggtgtgggagccattgtgctgccctttacgtttcgttggacgaagtcgtttacgctgatactgcaggcgttggatatgtacaacacatcctatccagatgcggagaggttaattgaggaaacatcatactcgggcgtgatactgccgtcgccggagtggaagacgctggaccacatcgggcggaacgcgcggatcacctaccgtgtccgggtgcaatgcgccgttacctactacaacacgacctgcacgaccttctgccgtccgcgggacgatcagttcggtcactacgcctgcggctccgagggtcagaagctctgcctgaatggctggcagggcgtcaactgcgaggaggccatatgcaaggcgggctgcgaccccgtccacggcaagtgcgatcgtccgggggaatgcgaatgcagacccggctggcgtggtccattgtgcaacgagtgcatggtctatcccggctgcaagcatggttcctgcaacggcagcgcctggaaatgcgtgtgcgacaccaactggggtggcatattgtgcgatcaagatttaaatttctgcggcacccatgaaccctgcaagcacggcggcacctgcgaaaataccgctccggacaaatatcggtgcacatgcgccgagggcctctcgggcgagcagtgcgagatcgtggagcacccatgtgccaccaggccatgccgcaacggcggcacatgcacactcaagacgagtaaccgaactcaagcccaagtgtatcgcacatcacatggcaggagcaacatgggccggccggtaagacgcagcagttcgatgcgcagcctggatcacctgcggccggaggggcaggcgctgaatggcagcagctcctcgggattggtgtccctaggttcgctgcagctgcagcagcaactggcccccgacttcacttgcgactgcgcagccggatggacgggaccgacatgcgaaataaatatcgacgagtgcgccgggggtccctgcgagcatggtggcacttgcatcgatctaatcggtggctttcgatgtgaatgtccgccggagtggcatggcgatgtctgtcaggtggatgtgaacgagtgcgaggcgccgcattccgccggaatcgctgcgaacgcattgctgaccaccacagccaccgcgattattggtagtaatctgagcagtactgctcttctggccgctctgaccagtgcagtggcatccacatccttggccatcggaccctgcatcaatgccaaggagtgtcgcaatcagccgggttcctttgcctgcatctgcaaggagggctggggcggagtgacctgtgccgagaatctagatgactgtgtgggtcagtgccggaatggagccacctgcattgatctggtcaacgactataggtgcgcctgtgcctctggattcacgggtcgcgattgcgagacggacatagacgagtgcgccacttccccgtgccgaaacggaggcgaatgtgtggacatggtgggcaaattcaattgcatttgcccacttggctactcgggttctctgtgcgaggaggccaaggagaactgcacaccgtcgccatgtttggagggtcactgcctcaacacgcccgaaggatactactgccattgtccaccggatcgcgccggaaagcactgcgagcaactgcgtccgctctgctcccagccgccctgcaacgagggctgcttcgccaatgtcagcctagcgacgtcagcgacaacgacgacgacaaccaccacaacggcgacaacgacaaggaagatggccaagccaagcggattgccctgcagcggacacggcagctgcgagatgagcgacgtgggcaccttctgcaaatgccatgtgggccacaccggcaccttctgcgagcacaatctcaacgaatgctcgccgaatccttgtcgaaatgggggaatttgccttgacggcgacggcgattttacatgcgagtgcatgtcgggctggacaggtaaacgctgctcggagcgcgctacaggttgttatgccggtcagtgccagaatggtggtacctgcatgcctggagccccggacaaggctctgcagccgcattgccgctgtgcgccaggttggactggtctgttttgcgccgaggctattgaccagtgtcgcgggcagccgtgccacaatggcggaacgtgcgagtcgggagcgggctggttccgctgcgtctgcgctcagggattctctggtccagactgccgcatcaatgtgaacgagtgctcgccacagccttgccagggcggtgccacctgcatcgacggaatcggtggatacagctgcatctgcccaccaggaaggcatggattgcggtgtgaaattttgctctccgatcccaagtccgcctgccagaacgcaagcaacactatctctccgtatacagctctaaaccgaagccaaaactggctggatattgctctaaccggaagaacagaagacgatgagaactgcaatgcgtgtgtctgcgaaaacggcacctctcggtgcacgaatctctggtgtggattgcccaattgctataaggtggatccgctctccaagtcctcgaatctgtccggtgtttgcaaacagcacgaggtgtgcgttccggcactgagtgagacatgcctgtcatcgccttgtaatgttcgtggagattgccgggcactggaaccatcgcgtcgggttgctccaccccgactgccagccaaatctagctgctggcccaatcaggccgtggtcaacgagaactgcgcccgactcaccatccttttggccctggagcgagtgggcaagggagcttcggtggagggtctctgctccctggtaagggtgctgctggctgcccagttgatcaagaagccggcgagtacttttggccaggatccgggaatgcttatggtgctctgcgatctcaaaacgggcaccaatgataccgttgaactaactgtgtcgtccagtaaattaaatgatccccagctgccagtggcggtgggtctgctgggtgaactcctgagctccaggcagttgaatggcatccagcggcgcaaggaactggagctgcagcatgcaaaattggctgccctcacctccattgtggaggtcaagttggaaacggcccgcgtggccgatggatcgggtcatagtctgctgataggagtgctatgcggtgtctttatagtcctggtgggattctcggtgttcatcagtctttactggaaacagcgtctggcttatcgcaccagttcgggaatgaacttaactccctccctggatgcactgcgtcacgaggaggagaagtcgaataatctgcagaacgaggagaatctgcgaaggtatacaaatccgctgaagggcagcaccagttccctaagagcggccaccggcatggaactaagcctcaatcccgctccggaattagccgcctcggcggcgagtagttccgccttgcacagatcgcagccactattcccgccatgcgatttcgagcgtgagctggactccagtacgggcctgaagcaggcgcacaagcggagctcacagattctgctgcacaaaacccaaaactcggacatgcggaagaacactgtgggctcgctggacagtccgcgtaaggactttggcaagcggtcgatcaactgcaagtccatgccaccctcttcgggcgacgagggctccgatgtccttgccaccactgtgatggtttagccgtgatctcaccaaccaaccaatcaagaaaccaaccagccgcccacagccagctcaaagttccaattgccacagcacgggcgctatttccaagtgcattagtagcgtaattaaaactaggatattgttaaggataccaaggtaggccacaacggagtggctctgttgaaaacgtaaagttctaaaaatccaggtctctcagacaaagatgaggtacacaaataaattggctagttaatcaagcatgttatggccacgggatgggcaaatttatttgtatacctgatcttatcttaatactaaaccagttttctactattttttttttgtggatcaagcttaaaagttcagctaggcaggcgttttccgcagtgccatgtcgatgtggaagcccaaaatatttaggttagatagtgtaatttcgaactcttctcttcgctaagcaacatcctacacagtgtgatatttagtgtaacccaggcgcgcatttacattcaattaaagacaatgatatataaatataaacgaaatcaactccttggctagcacaagctgtatgtatatagttctcatttaggatcgtcgcgctctatattgtgtataagctgtaaatactgtaaattagcagttaccgttattgtattttgtctatagttagattggtactattaaactaagaaccagccgcaacgcgttagactttaaaagttgtttgcaattgtacgcaataatatagttttatgctcgtagttaggtagctgtgtaaccgggtaagattcaaacgattttgtactgtattatatacctatctgtgtagtaatatttatttattatattaaatttgatctagacgcaataaagtaatatcaataaagatagtaaaagacat

SEQ ID NO: 2 - protein, 1404 aa, Drosophila
  Drosophila Serrate (Ser)
MFRKHFRRKPATSSSLESTIESADSLGMSKKTATKRQRPRHRVPKIATLPSTIRDCRSLKSACNLIALILILLVHKISAAGNFELEILEISNTNSHLLNGYCCGMPAELRATKTIGCSPCTTAFRLCLKEYQTTEQGASISTGCSFGNATTKILGGSSFVLSDPGVGAIVLPFTFRWTKSFTLILQALDMYNTSYPDAERLIEETSYSGVILPSPEWKTLDHIGRNARITYRVRVQCAVTYYNTTCTTFCRPRDDQFGHYACGSEGQKLCLNGWQGVNCEEAICKAGCDPVHGKCDRPGECECRPGWRGPLCNECMVYPGCKHGSCNGSAWKCVCDTNWGGILCDQDLNFCGTHEPCKHGGTCENTAPDKYRCTCAEGLSGEQCEIVEHPCATRPCRNGGTCTLKTSNRTQAQVYRTSHGRSNMGRPVRRSSSMRSLDHLRPEGQALNGSSSSGLVSLGSLQLQQQLAPDFTCDCAAGWTGPTCEINIDECAGGPCEHGGTCIDLIGGFRCECPPEWHGDVCQVDVNECEAPHSAGIAANALLTTTATAIIGSNLSSTALLAALTSAVASTSLAIGPCINAKECRNQPGSFACICKEGWGGVTCAENLDDCVGQCRNGATCIDLVNDYRCACASGFTGRDCETDIDECATSPCRNGGECVDMVGKFNCICPLGYSGSLCEEAKENCTPSPCLEGHCLNTPEGYYCHCPPDRAGKHCEQLRPLCSQPPCNEGCFANVSLATSATTTTTTTTTATTTRKMAKPSGLPCSGHGSCEMSDVGTFCKCHVGHTGTFCEHNLNECSPNPCRNGGICLDGDGDFTCECMSGWTGKRCSERATGCYAGQCQNGGTCMPGAPDKALQPHCRCAPGWTGLFCAEAIDQCRGQPCHNGGTCESGAGWFRCVCAQGFSGPDCRINVNECSPQPCQGGATCIDGIGGYSCICPPGRHGLRCEILLSDPKSACQNASNTISPYTALNRSQNWLDIALTGRTEDDENCNACVCENGTSRCTNLWCGLPNCYKVDPLSKSSNLSGVCKQHEVCVPALSETCLSSPCNVRGDCRALEPSRRVAPPRLPAKSSCWPNQAVVNENCARLTILLALERVGKGASVEGLCSLVRVLLAAQLIKKPASTFGQDPGMLMVLCDLKTGTNDTVELTVSSSKLNDPQLPVAVGLLGELLSSRQLNGIQRRKELELQHAKLAALTSIVEVKLETARVADGSGHSLLIGVLCGVFIVLVGFSVFISLYWKQRLAYRTSSGMNLTPSLDALRHEEEKSNNLQNEENLRRYTNPLKGSTSSLRAATGMELSLNPAPELAASAASSSALHRSQPLFPPCDFERELDSSTGLKQAHKRSSQILLHKTQNSDMRKNTVGSLDSPRKDFGKRSINCKSMPPSSGDEGSDVLATTVMV

SEQ ID NO: 3 - DNA, 5590 bp, Homo sapiens
  human Serrate-1
ctgcggccggcccgcgagctaggctgggtttttttttttctcccctccctcccccctttttccatgcagctgatctaaaagggaataaaaggctgcgcataatcataataataaaagaaggggagcgcgagagaaggaaagaaagccgggaggtggaagaggagggggagcgtctcaaagaagcgatcagaataataaaaggaggccgggctctttgccttctggaacgggccgctcttgaaagggcttttgaaaagtggtgttgttttccagtcgtgcatgctccaatcggcggagtatattagagccgggacgcggcggccgcaggggcagcggcgacggcagcaccggcggcagcaccagcgcgaacagcagcggcggcgtcccgagtgcccgcggcgcgcggcgcagcgatgcgttccccacggacgcgcggccggtccgggcgccccctaagcctcctgctcgccctgctctgtgccctgcgagccaaggtgtgtggggcctcgggtcagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccgaggtcctatacgttgcttgtggaggcgtgggattccagtaatgacaccgttcaacctgacagtattattgaaaaggcttctcactcgggcatgatcaaccccagccggcagtggcagacgctgaagcagaacacgggcgttgcccactttgagtatcagatccgcgtgacctgtgatgactactactatggctttggctgcaataagttctgccgccccagagatgacttctttggacactatgcctgtgaccagaatggcaacaaaacttgcatggaaggctggatgggccccgaatgtaacagagctatttgccgacaaggctgcagtcctaagcatgggtcttgcaaactcccaggtgactgcaggtgccagtatggctggcaaggcctgtactgtgataagtgcatcccacacccgggatgcgtccacggcatctgtaatgagccctggcagtgcctctgtgagaccaactggggcggccagctctgtgacaaagatctcaattactgtgggactcatcagccgtgtctcaacgggggaacttgtagcaacacaggccctgacaaatatcagtgttcctgccctgaggggtattcaggacccaactgtgaaattgctgagcacgcctgcctctctgatccctgtcacaacagaggcagctgtaaggagacctccctgggctttgagtgtgagtgttccccaggctggaccggccccacatgctctacaaacattgatgactgttctcctaataactgttcccacgggggcacctgccaggacctggttaacggatttaagtgtgtgtgccccccacagtggactgggaaaacgtgccagttagatgcaaatgaatgtgaggccaaaccttgtgtaaacgccaaatcctgtaagaatctcattgccagctactactgcgactgtcttcccggctggatgggtcagaattgtgacataaatattaatgactgccttggccagtgtcagaatgacgcctcctgtcgggatttggttaatggttatcgctgtatctgtccacctggctatgcaggcgatcactgtgagagagacatcgatgaatgtgccagcaacccctgtttgaatgggggtcactgtcagaatgaaatcaacagattccagtgtctgtgtcccactggtttctctggaaacctctgtcagctggacatcgattattgtgagcctaatccctgccagaacggtgcccagtgctacaaccgtgccagtgactatttctgcaagtgccccgaggactatgagggcaagaactgctcacacctgaaagaccactgccgcacgaccccctgtgaagtgattgacagctgcacagtggccatggcttccaacgacacacctgaaggggtgcggtatatttcctccaacgtctgtggtcctcacgggaagtgcaagagtcagtcgggaggcaaattcacctgtgactgtaacaaaggcttcacgggaacatactgccatgaaaatattaatgactgtgagagcaacccttgtagaaacggtggcacttgcatcgatggtgtcaactcctacaagtgcatctgtagtgacggctgggagggggcctactgtgaaaccaatattaatgactgcagccagaacccctgccacaatgggggcacgtgtcgcgacctggtcaatgacttctactgtgactgtaaaaatgggtggaaaggaaagacctgccactcacgtgacagtcagtgtgatgaggccacgtgcaacaacggtggcacctgctatgatgagggggatgcttttaagtgcatgtgtcctggcggctgggaaggaacaacctgtaacatagcccgaaacagtagctgcctgcccaacccctgccataatgggggcacatgtgtggtcaacggcgagtcctttacgtgcgtctgcaaggaaggctgggaggggcccatctgtgctcagaataccaatgactgcagccctcatccctgttacaacagcggcacctgtgtggatggagacaactggtaccggtgcgaatgtgccccgggttttgctgggcccgactgcagaataaacatcaatgaatgccagtcttcaccttgtgcctttggagcgacctgtgtggatgagatcaatggctaccggtgtgtctgccctccagggcacagtggtgccaagtgccaggaagtttcagggagaccttgcatcaccatggggagtgtgataccagatggggccaaatgggatgatgactgtaatacctgccagtgcctgaatggacggatcgcctgctcaaaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataatcgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattacgagaacaagaactccaaaatgtctaaaataaggacacacaattctgaagtagaagaggacgacatggacaaacaccagcagaaagcccggtttgccaagcagccggcgtatacgctggtagacagagaagagaagccccccaacggcacgccgacaaaacacccaaactggacaaacaaacaggacaacagagacttggaaagtgcccagagcttaaaccgaatggagtacatcgtatagcagaccgcgggcactgccgccgctaggtagagtctgagggcttgtagttctttaaactgtcgtgtcatactcgagtctgaggccgttgctgacttagaatccctgtgttaatttaagttttgacaagctggcttacactggcaatggtagtttctgtggttggctgggaaatcgagtgccgcatctcacagctatgcaaaaagctagtcaacagtaccctggttgtgtgtccccttgcagccgacacggtctcggatcaggctcccaggagcctgcccagccccctggtctttgagctcccacttctgccagatgtcctaatggtgatgcagtcttagatcatagttttatttatatttattgactcttgagttgtttttgtatattggttttatgatgacgtacaagtagttctgtatttgaaagtgcctttgcagctcagaaccacagcaacgatcacaaatgactttattatttatttttttaattgtatttttgttgttgggggaggggagactttgatgtcagcagttgctggtaaaatgaagaatttaaagaaaaaaatgtcaaaagtagaactttgtatagttatgtaaataattcttttttattaatcactgtgtatatttgatttattaacttaataatcaagagccttaaaacatcattcctttttatttatatgtatgtgtttagaattgaaggtttttgatagcattgtaagcgtatggctttatttttttgaactcttctcattacttgttgcctataagccaaaattaaggtgtttgaaaatagtttattttaaaacaataggatgggcttctgtgcccagaatactgatggaattttttttgtacgacgtcagatgtttaaaacaccttctatagcatcacttaaaacacgttttaaggactgactgaggcagtttgaggattagtttagaacaggtttttttgtttgtttgttttttgtttttctgctttagacttgaaaagagacaggcaggtgatctgctgcagagcagtaagggaacaagttgagctatgacttaacatagccaaaatgtgagtggttgaatatgattaaaaatatcaaattaattgtgtgaacttggaagcacaccaatctgactttgtaaattctgatttcttttcaccattcgtacataatactgaaccacttgtagatttgattttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttgtattgtcctattagtgttatatgaacatacaaatgcatctttgatgtgttgttcttggcaataaattttgaaaagtaatatttattaaatttttttgtatgaaaac

SEQ ID NO: 4 - protein, 1218 aa, Homo sapiens
  human Serrate-1
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 5 - DNA, 4702 bp, Homo sapiens
  human Serrate-2
ggccggggccgggcgggcgggtcgcgggggcaatgcgggcgcagggccgggggcgccttccccggcggctgctgctgctgctggcgctctgggtgcaggcggcgcggcccatgggctatttcgagctgcagctgagcgcgctgcggaacgtgaacggggagctgctgagcggcgcctgctgtgacggcgacggccggacaacgcgcgcggggggctgcggccacgacgagtgcgacacgtacgtgcgcgtgtgccttaaggagtaccaggccaaggtgacgcccacggggccctgcagctacggccacggcgccacgcccgtgctgggcggcaactccttctacctgccgccggcgggcgctgcgggggaccgagcgcgggcgcgggcccgggccggcggcgaccaggacccgggcctcgtcgtcatccccttccagttcgcctggccgcgctcctttaccctcatcgtggaggcctgggactgggacaacgataccaccccgaatgaggagctgctgatcgagcgagtgtcgcatgccggcatgatcaacccggaggaccgctggaagagcctgcacttcagcggccacgtggcgcacctggagctgcagatccgcgtgcgctgcgacgagaactactacagcgccacttgcaacaagttctgccggccccgcaatgactttttcggccactacacctgcgaccagtacggcaacaaggcctgcatggacggctggatgggcaaggagtgcaaggaagctgtgtgtaaacaagggtgtaatttgctccacgggggatgcaccgtgcctggggagtgcaggtgcagctacggctggcaagggaggttctgcgatgagtgtgtcccctaccccggctgcgtgcatggcagttgtgtggagccctggcagtgcaactgtgagaccaactggggcggcctgctctgtgacaaagacctgaactactgtggcagccaccacccctgcaccaacggaggcacgtgcatcaacgccgagcctgaccagtaccgctgcacctgccctgacggctactcgggcaggaactgtgagaaggctgagcacgcctgcacctccaacccgtgtgccaacgggggctcttgccatgaggtgccgtccggcttcgaatgccactgcccatcgggctggagcgggcccacctgtgcccttgacatcgatgagtgtgcttcgaacccgtgtgcggccggtggcacctgtgtggaccaggtggacggctttgagtgcatctgccccgagcagtgggtgggggccacctgccagctggacgccaatgagtgtgaagggaagccatgccttaacgctttttcttgcaaaaacctgattggcggctattactgtgattgcatcccgggctggaagggcatcaactgccatatcaacgtcaacgactgtcgcgggcagtgtcagcatgggggcacctgcaaggacctggtgaacgggtaccagtgtgtgtgcccacggggcttcggaggccggcattgcgagctggaacgagacaagtgtgccagcagcccctgccacagcggcggcctctgcgaggacctggccgacggcttccactgccactgcccccagggcttctccgggcctctctgtgaggtggatgtcgacctttgtgagccaagcccctgccggaacggcgctcgctgctataacctggagggtgactattactgcgcctgccctgatgactttggtggcaagaactgctccgtgccccgcgagccgtgccctggcggggcctgcagagtgatcgatggctgcgggtcagacgcggggcctgggatgcctggcacagcagcctccggcgtgtgtggcccccatggacgctgcgtcagccagccagggggcaacttttcctgcatctgtgacagtggctttactggcacctactgccatgagaacattgacgactgcctgggccagccctgccgcaatgggggcacatgcatcgatgaggtggacgccttccgctgcttctgccccagcggttgggagggcgagctctgcgacaccaatcccaacgactgccttcccgatccctgccacagccgcggccgctgctacgacctggtcaatgacttctactgtgcgtgcgacgacggctggaagggcaagacctgccactcacgcgagttccagtgcgatgcctacacctgcagcaacggtggcacctgctacgacagcggcgacaccttccgctgcgcctgcccccccggctggaagggcagcacctgcgccgtcgccaagaacagcagctgcctgcccaacccctgtgtgaatggtggcacctgcgtgggcagcggggcctccttctcctgcatctgccgggacggctgggagggtcgtacttgcactcacaataccaacgactgcaaccctctgccttgctacaatggtggcatctgtgttgacggcgtcaactggttccgctgcgagtgtgcacctggcttcgcggggcctgactgccgcatcaacatcgacgagtgccagtcctcgccctgtgcctacggggccacgtgtgtggatgagatcaacgggtatcgctgtagctgcccacccggccgagccggcccccggtgccaggaagtgatcgggttcgggagatcctgctggtcccggggcactccgttcccacacggaagctcctgggtggaagactgcaacagctgccgctgcctggatggccgccgtgactgcagcaaggtgtggtgcggatggaagccttgtctgctggccggccagcccgaggccctgagcgcccagtgcccactggggcaaaggtgcctggagaaggccccaggccagtgtctgcgaccaccctgtgaggcctggggggagtgcggcgcagaagagccaccgagcaccccctgcctgccacgctccggccacctggacaataactgtgcccgcctcaccttgcatttcaaccgtgaccacgtgccccagggcaccacggtgggcgccatttgctccgggatccgctccctgccagccacaagggctgtggcacgggaccgcctgctggtgttgctttgcgaccgggcgtcctcgggggccagtgctgtggaggtggccgtgtccttcagccctgccagggacctgcctgacagcagcctgatccagggcgcggcccacgccatcgtggccgccatcacccagcgggggaacagctcactgctcctggctgtcaccgaggtcaaggtggagacggttgttacgggcggctcttccacaggtctgctggtgcctgtgctgtgtggtgccttcagcgtgctgtggctggcgtgcgtggtcctgtgcgtgtggtggacacgcaagcgcaggaaagagcgggagaggagccggctgccgcgggaggagagcgccaacaaccagtgggccccgctcaaccccatccgcaaccccattgagcggccgggggggcacaaggacgtgctctaccagtgcaagaacttcacgccgccgccgcgcagggcggacgaggcgctgcccgggccggccggccacgcggccgtcagggaggatgaggaggacgaggatcttggccgcggtgaggaggactccctggaggcggagaagttcctctcacacaaattcaccaaagatcctggccgctcgccggggaggccggcccactgggcctcaggccccaaagtggacaaccgcgcggtcaggagcatcaatgaggcccgctacgtcggcaaggaatagggcggctgcagctgggccgggacccagggccctcggtgggagccatgccgtctgccggacccggaggccgaggccatgtgcatagtttctttattttgtgtaaaaaaaccaccaaaaacaaaaaccaaatgtttattttctacgtttctttaaccttgtataaattattcagtaactgtcaggctgaaaacaatggagtattctcggatagttgctatttttgtaaagtagccgtgcgtggcactcgctgtatgaaaggagagagcaaagggtgtctgcgtcgtcaccaaatcgtcgcgtttgttaccagaggttgtgcactgtttacagaatcttccttttattcctcactcgggtttctctgtgctccaggccaaagtgccggtgagacccatggctgtgttggtgtggcccatggctgttggtgggacccgtggctgatggtgtggcctgtggctgtcggtgggactcgtggctgtcaatgggacctgtggctgtcggtgggacctacggtggtcggtgggaccctggttattgatgtggccctggctgccggcacggcccgtggctgttgacgcacctgtggttgttagtggggcctgaggtcatcggcgtggcccaaggccggcaggtcaacctcgcgcttgctggccagtccaccctgcctgccgtctgtgcttcctcctgcccagaacgcccgctccagcgatctctccactgtgctttcagaagtgcccttcctgctgcgcagttctcccatcctgggacggcggcagtattgaagctcgtgacaagtgccttcacacagacccctcgcaactgtccacgcgtgccgtggcaccaggcgctgcccacctgccggccccggccgcccctcctcgtgaaagtgcatttttgtaaatgtgtacatattaaaggaagcactctgtataaaaaaaaaaaaccggaattcc

SEQ ID NO: 6 - protein, 1238 aa, Homo sapiens [sequence omitted]

SEQ ID NO: 7 - protein, 58 aa, Artificial Sequence
  Sequence removed from ELR4 for alignment
AQVVRTSHGRSNMGRPVRRSSSMRSLDHLRPEGQALNGSSSSGLVLGSLGLGGGLAPD

SEQ ID NO: 8 - protein, 44 aa, Artificial Sequence
  sequence removed from ELR6 for alignment
HSAGIAANALLTTTATAIIGSNLSSTALLAALTSAVASTSLAIG

SEQ ID NO: 9 - DNA, 45 bp, Artificial Sequence
  primer used to create an in frame Dra I site at the beginning of EGF-like repeat 4
cgagatcgtttaaatttctgtgccaccaggccatgccgcaacggc

SEQ ID NO: 10 - DNA, 39 bp, Artificial Sequence
  primer for placing an artificial Ppu MI site at position 1619 of the Ser cDNA
gccgccgttgcggcagggacccgtggcacatgggtgctc

SEQ ID NO: 11 - DNA, 45 bp, Artificial Sequence
  primer for placing an artificial Kas I site at position 1916 of the Ser cDNA
gccaccatgctcgcagggcggcgcctcgcactcgtcgatatttat

SEQ ID NO: 12 - DNA, 30 bp, Artificial Sequence
  primer for placing an artificial Kas I site at position 2277 of the Ser cDNA
gactgtgtggcgccgtgccggaatggagcc

SEQ ID NO: 13 - DNA, 37 bp, Artificial Sequence
  primer used to introduce a unique Sac I site at position 2029
ccggatcgatgcgggagctcgcactcgttcacatcca

SEQ ID NO: 14 - DNA, 33 bp, Artificial Sequence
  primer used to introduce a unique Sac I site at position 2161
cacatccttggagctcggaccctgcatcaatgc

SEQ ID NO: 15 - DNA, 33 bp, Artificial Sequence
  primer used to placing an artificial Xba I site at position 2373
gcgagacggatctagacgagtgcgccacttccc

SEQ ID NO: 16 - DNA, 43 bp, Artificial Sequence
  N-terminal encoding primer used to generate the Nterm6 construct
tgccgagaatctagatgacatggtgagcaagggcgaggaggtc

SEQ ID NO: 17 - DNA, 36 bp, Artificial Sequence
  C-terminal encoding primer used to generate the Nterm6 construct
tgatcggaattcttacttgtacagctcgtccatgcc

SEQ ID NO: 18 - DNA, 39 bp, Artificial Sequence
  primer used to introduc Bgl II site into the N-terminal region of Ser
ggtatttgagatttctaagatctccagctcgaagttacc

SEQ ID NO: 19 - DNA, 39 bp, Artificial Sequence
  primer used to generate a second Bgl II site
tgcgagatcgtggagcagatctgtgccaccaggccatgc

SEQ ID NO: 20 - DNA, 30 bp, Artificial Sequence
  N-terminal primer used to generate tomato tag
gctccggaaatggtgagcaagggcgaggag

SEQ ID NO: 21 - DNA, 33 bp, Artificial Sequence
  C-terminal primer used to generate tomato tag
taattccggagccttgtacagctcgtccatgcc

SEQ ID NO: 22 - protein, 41 aa, Artificial Sequence
  Drosophila Serrate ELR4
CATRPCRNGGTCTLKTSNRTQFTCDCAAGWTGPTCEINIDE

SEQ ID NO: 23 - protein, 38 aa, Homo sapiens
  Human Jagged 1 (Jagged-1-1)
CLSDPCHNRGSCKETSLGFECECSPGWTGPTCSTNIDD

SEQ ID NO: 24 - protein, 38 aa, Chicken
  Chicken Serrate 1 (C-Serrate-1)
CLSDPCHNGGSCLETSTGFECVCAPGWAGPTCTDNIDD

SEQ ID NO: 25 - protein, 38 aa, Xenopus
  Xenopus Jagged 1 (X-Jagged-1)
CLSDPCHNGGSCLETSVGFECQCARGWTGPTCSINIDE

SEQ ID NO: 26 - protein, 38 aa, Zebrafish
  Zebrafish Jagged 1 (Jagged-1a)
CLSEPCLNGGLCVESSLGFECQCAAGWTGPSCNINEDD

SEQ ID NO: 27 - protein, 43 aa, Drosophila
  Drosophila Delta
CDADVNPCQNGGTCIDEPHTKTGYKCHCANGWSGKMCEEKVLT

SEQ ID NO: 28 - protein, 38 aa, Artificial Sequence
  Drosophila Serrate ELR5
CAGGPCEHGGTCIDLIGGFRCECPPEWHGDVCQVDVNE

SEQ ID NO: 29 - protein, 38 aa, Homo sapiens
  Human Jagged 1 (Jagged-1-1)
CSPNNCSHGGTCQDLVNGFKCVCPPQWTGKTCQLDANE

SEQ ID NO: 30 - protein, 38 aa, Chicken
  Chicken Serrate 1 (C-Serrate-1)
CSPNPCGHGGTCQDLVDGFKCICPPQWTGKTCQLDANE

SEQ ID NO: 31 - protein, 38 aa, Xenopus
  Xenopus Jagged 1 (X-Jagged-1)
CSPNPCGYGGTCQDLIDGFKCICPSQWTGKTCQIDANE

SEQ ID NO: 32 - protein, 38 aa, Zebrafish
  Zebrafish Jagged 1 (Jagged-1a)
CSPNPCNHSGVCVDLVDGFKCICPVQWTGKTCLIDANE

SEQ ID NO: 33 - protein, 44 aa, Drosophila
  Drosophila Delta
CSDKPCHQGICRNVRPGLGSKGQGYQCECPIGYSGPNCDLQLDN

SEQ ID NO: 34 - protein, 38 aa, Artificial Sequence
  Drosophila Serrate ELR6
CEAPPCINAKECRNQPGSFACICKEGWGGVTCAENLDD

SEQ ID NO: 35 - protein, 38 aa, Homo sapiens
  Human Jagged 1 (Jagged-1-1)
CEAKPCVNAKSCKNLIASYYCDCLPGWMGQNCDININD

SEQ ID NO: 36 - protein, 38 aa, Chicken
  Chicken Serrate 1 (C-Serrate-1)
CEGKPCVNANSCRNLIGSYYCDCITGWSGHNCDININD

SEQ ID NO: 37 - protein, 38 aa, Xenopus
  Xenopus Jagged 1 (X-Jagged-1)
CEAKPCVNANSCRNLIGSYYCNCLPGWTGQNCDININD

SEQ ID NO: 38 - protein, 38 aa, Zebrafish
  Zebrafish Jagged 1 (Jagged-1a)
CEESPCVNAHSCRNLIGGYFCECLPGWTGQNCDINVND

SEQ ID NO: 39 - protein, 35 aa, Drosophila
  Drosophila Delta
CSPNPCINGGSCQPSGKCICPAGFSGTRCETNIDD

(19) United States
(12) Patent Application Publication
(10) Pub. No.: US 2015/0291681 A1
(43) Pub. Date: Oct. 15, 2015

(54) METHODS AND COMPOSITIONS FOR MODULATING NOTCH ACTIVITY

(71) Applicants: Robert J. Fleming, Manchester, CT (US); Spyridon Artavanis-Tsakonas, Brookline, MA (US)

(72) Inventors: Robert J. Fleming, Manchester, CT (US); Spyridon Artavanis-Tsakonas, Brookline, MA (US)

(21) Appl. No.: 14/388,598

(22) PCT Filed: Mar. 29, 2012
(86) PCT No.: PCT/US12/31213

§ 371 Date: Mar. 19, 2015

(51) Int. Cl. C07K 14/705; C07K 16/28

(52) U.S. Cl. CPC C07K 14/705; C07K 16/28; C07K 2319/30; A61K 38/00

(57) ABSTRACT

Provided herein are polypeptides and derivatives thereof that contain sequences of a Serrate protein and that inhibit Notch pathway function. Also provided herein are nucleic acids that encode the polypeptides, vectors and cells for producing the polypeptides, and related pharmaceutical compositions and kits. Additionally provided are antibodies which bind to the Notch inhibitory region of a Serrate protein. Therapeutic methods are also provided.

### 1. INTRODUCTION

The present invention relates to Serrate protein polypeptides and nucleic acids capable of inhibiting Notch signaling. Methods of producing the Serrate protein polypeptides are also provided. The invention further relates to methods of therapy and therapeutic compositions and kits.

### 2. BACKGROUND

In the development of multicellular organisms, the Notch signaling pathway is a fundamental mechanism that controls cell fates and, consequently, morphogenesis. This highly evolutionarily conserved signaling pathway is essential for proper embryonic development in all metazoan organisms and profoundly affects differentiation, proliferation and apoptotic events throughout development (Lai, 2004, Development 5:965-973). The central element of the pathway, the Notch receptor, transmits signals that are pleiotropic and affect the expression of hundreds of genes in a context-dependent manner (Louvi and Artavanis-Tsakonas, 2012, Semin. Cell Dev. Biol. [Epub ahead of print], PMID:22373641 [PubMed]).

Notch signaling regulates binary cell fate decisions in precursor cells and boundary formation between cell populations. This regulation requires direct interaction between cells. Therefore, Notch signaling controls cellular fates and the segregation of lineages by linking the fate of one cell to that of a neighbor through the interaction of the Notch surface receptor expressed on one cell with membrane bound ligands expressed on the surface of an adjacent cell (Louvi and Artavanis-Tsakonas, 2012, Semin. Cell Dev. Biol. [Epub ahead of print], PMID:22373641 [PubMed]).

The Notch receptor was first cloned and characterized in Drosophila, which has a single receptor (Wharton et al., 1985, Cell 43:567-581). The paradigmatic Drosophila Notch is composed of distinct domains that are essentially conserved across all species. Four Notch paralogues (designated Notchl-4) have been identified in mammals and display differences in primary sequence. However, they do have overlapping, yet individual, expression profiles and developmental functions, even though likely interchangeable biochemical functions (Artavanis-Tsakonas et al., 1999, Science 284:770-776; Kopan and Ilagan, 2009, Cell 137:216-233).

Notch family receptors are large single-pass type I transmembrane proteins, with a functional extracellular domain, a transmembrane domain, and an intracellular domain (NICD). The extracellular domain contains multiple epidermal growth factor (EGF)-like repeats (ELRs) followed by a negative regulatory region, and the intracellular region contains the RAM domain, ankyrin repeats, and a C-terminal PEST domain. For example, the Drosophila Notch protein is approximately 2,700 amino acids in length with a 1,700-aa, extracellular, cysteine-rich domain harboring 36 tandem ELRs (Wharton et al., 1985, Cell 43:567-581). The canonical signaling model has the Notch receptor being activated through a series of proteolytic events after it interacts with the ligands Delta (Dl) or Serrate (Ser) (also called Jagged in vertebrates) (Kopan and Ilagan, 2009, Cell 137:216-233; Bray, 2006, Nat. Rev. Mol. Cell Biol. 7:678-689). The crucial cleavage event for signaling depends on γ-secretase and results in releasing the NICD from the membrane. NICD then translocates to the nucleus, complexes with CSL (CBF1, RBPjκ, Suppressor of Hairless (Su(H)), Lag-1), displaces a histone deacetylase (HDAc)-co-repressor (CoR) complex, and recruits components of an activation complex, such as MAML1 and histone acetyltransferases (HAc), resulting in activation of gene transcription (Kopan and Ilagan, 2009, Cell 137:216-233; Bray, 2006, Nat. Rev. Mol. Cell Biol. 7:678-689; Bray and Furriols, 2001, Curr. Biol. 6:R217-221; Petcherski and Kimble, 2000, Nature 6784:364-368). Well-established target genes of this transcription activation complex include the Hes and Hey gene family.

Active Notch signals are usually generated when a ligand-expressing (sending cell) contacts Notch on an adjacent receiving cell. During this event, there are many cellular processes that regulate signal transmission. The prevailing notion is that in the sending cell, ligand endocytosis is required to trigger Notch activation in the receiving cell (Overstreet et al., 2004, Development 21:5355-5366; Parks et al., 2000, Development 7:1373-1385; Seugnet et al., 1997, Dev. Biol. 2:585-598; Le Borgne, 2006, Curr. Opin. Cell Biol. 2:213-222), an event associated with ligand monoubiquitination by neuralized or mindbomb (Le Borgne et al., 2005, Development 8:1751-1762; Wang and Struhl, 2005, Development 12:2883-2894). Cells deficient in specific endocytic components such as dynamin and epsin are incapable of signaling to an adjacent Notch expressing cell (Wang and Struhl, 2005, Development 12:2883-2894). In receiving cells, Notch molecules are apparently cleaved proteolytically by a furin-like protease prior to being placed in the cell membrane as a heteromeric receptor (Kidd and Lieber, 2002, Mech. Dev. 1-2:41-51; Logeat et al., 1998, Proc. Natl. Acad. Sci. U.S.A. 14:8108-8112). The interaction of Delta or Ser-like ligands with the Notch receptor is also regulated by modification of Notch by the fringe (fng) family of glycosyltransferases (Bruckneret al., 2000, Nature 6794:411-415; Moloney et al., 2000, Nature 6794:369-375). When Notch is modified by fng in Drosophila, it is preferentially activated by Delta but not Ser, providing a ligand-specific Notch response (Panin et al., 1997, Nature 6636:908-912). The steady state level of Notch receptor on cell surfaces is regulated by several ubiquitin ligases, the intracellular PEST domain and interactions with regulatory proteins such as Numb and α-adaptin (Le Borgne, 2006, Curr. Opin. Cell Biol. 2:213-222). All of the aforementioned regulatory events control Notch activity during trans-activation by its ligands. However, an important part of the intricate control of the Notch signaling pathway relies on the ability of the ligands to repress Notch activity when the receptor and ligand are co-expressed on the same cell (de Celis and Bray, 1997, Development 17:3241-3251; Micchelli et al., 1997, Development 8:1485-1495; del Alamo et al., 2011, Curr. Biol. 1:R40-47). This interaction, termed cis-inhibition, is highly dependent on the relative concentrations of ligand and receptor and remains enigmatic. Notch interactions with ligand generate a graded, activation response to ligand levels in trans but has a sharper, threshold type of inhibitory response to ligand interactions in cis (Sprinzak et al., 2010, Nature 7294:86-90). These differences are likely to be essential to signal directionality, particularly in regions where cells initially express both ligand and receptor as they establish signal-sending and signal-receiving cell types. An example is the case in the neurogenic region of the Drosophila ectoderm where Notch sending versus receiving cells are segregated from a field of developmentally equivalent cells expressing both the receptor and the ligand.

Inhibition of the Notch receptor by its ligands has also been reported in a non-cis-inhibitory context. Notch ligands lacking only the intracellular domain or lacking both the intracellular and transmembrane domains (i.e. secreted ligand forms) lose the ability to trans-activate Notch but retain strong inhibitory interactions with the receptor (Hukriede et al., 1997, Development 17:3427-3437; Hukriede and Fleming, 1997, Genetics 2:359-374; Sun and Artavanis-Tsakonas, 1997, Development 17:3439-3448; Sun and Artavanis-Tsakonas, 1996, Development 8:2465-2474). It has been reported that in Drosophila Ser demonstrates stronger cis-inhibitory properties than does Delta (Klein et al., 1997, Dev. Biol. 1:123-134; Li and Baker, 2004, BMC Dev. Biol. 5).

### 3. SUMMARY OF THE INVENTION

Provided herein are polypeptides and derivatives thereof that contain sequences of a Serrate protein and that inhibit Notch pathway function. Also provided herein are nucleic acids that encode the polypeptides and derivatives, vectors and cells for producing the polypeptides, and related pharmaceutical compositions and kits. Additionally provided are antibodies and fragments which specifically bind the therapeutic polypeptides and derivatives of the invention. Therapeutic methods are also provided.

The terms “Serrate” and “Jagged” are used interchangeably in this application, with Serrate therefore intended to encompass both mammalian Jagged and non-mammalian forms of the protein/gene.

In one embodiment, provided herein is a polypeptide comprising the DSL domain, the DOS domain, and the ELR4-6 domains of a Serrate protein, wherein said polypeptide lacks at least one of the following domains of said Serrate protein: ELR3, ELR7, ELR8, ELR9, ELR10, ELR11, or ELR12. In one embodiment, provided herein is a polypeptide comprising the amino terminus through the DOS domain of a Serrate protein, and the ELR4-6 domains of said Serrate protein, wherein said polypeptide lacks at least one of the following domains of said Serrate protein: ELR3, ELR7, ELR8, ELR9, ELR10, ELR11, or ELR12. In one embodiment, the polypeptide described herein lacks the ELR3 domain of said Serrate protein. In a specific embodiment, the polypeptide lacks each of the ELR3, ELR7, ELR8, ELR9, ELR10, ELR11, and ELR12 domains of said Serrate protein. In another embodiment, the polypeptide described herein lacks at least one of the following domains of said Serrate protein: ELR7, ELR8, ELR9, ELR10, ELR11, or ELR12. In a specific embodiment, the polypeptide lacks each of the ELR7, ELR8, ELR9, ELR10, ELR11, and ELR12 domains of said Serrate protein. In one embodiment, the polypeptide described herein lacks all ELRs except ELR4-6 of said Serrate protein. In another embodiment, the polypeptide described herein lacks all ELRs except ELR3-6 of said Serrate protein. In one embodiment, the polypeptide described herein further comprises ELR3 of said Serrate protein. In another embodiment, the polypeptide comprises a fragment of said Serrate protein that spans the amino terminus through ELR6, ELR7, ELR8, ELR9, ELR10 or ELR11. In one embodiment, the polypeptide described herein lacks the transmembrane domain and the intracellular domain of said Serrate protein.

In a specific embodiment, provided herein is a polypeptide consisting of the DSL domain, the DOS domain, and the ELR4-6 domains of a Serrate protein. In another embodiment, provided herein is a polypeptide consisting the DSL domain, the DOS domain, and the ELR3-6 domains of a Serrate protein.

In one embodiment, the Serrate protein described herein is a mammalian Serrate protein. In a specific embodiment, the mammalian Serrate protein is a human Serrate protein. In a specific embodiment, the human Serrate protein is human Serrate-1. In another embodiment, the human Serrate protein is human Serrate-2.

In one embodiment, the polypeptide described herein further comprises a non-Serrate protein amino acid sequence. In a specific embodiment, the non-Serrate protein amino acid sequence is of an immunoglobulin Fc (IgFc) domain. In one aspect, the invention relates to a derivative of the polypeptide described herein, wherein 1-5 conservative amino acid substitutions are present in the Serrate protein sequence of said polypeptide. In one embodiment, the polypeptide or derivative described herein is purified.

In one aspect, the invention relates to a nucleic acid that encodes the polypeptide or derivative thereof described herein. In one embodiment, the nucleic acid is a purified nucleic acid. In another aspect, the invention relates to a vector comprising a nucleotide sequence encoding the polypeptide or derivative thereof described herein operably linked to a promoter.

In one aspect, the invention relates to a host cell comprising the vector of the invention. In another aspect, the present invention relates to a host cell that expresses a polypeptide or derivative described herein. In one embodiment, the host cell expresses the polypeptide or derivative described herein from a transgene. In one embodiment, the host cell of this invention is an isolated host cell. In one aspect, the invention relates to a method of producing the polypeptide or derivative of this invention, comprising culturing a host cell comprising a recombinant nucleic acid encoding the polypeptide or derivative thereof operably linked to a promoter under conditions such that the polypeptide or derivative is produced by said host cell. In another aspect, the invention relates to a method of producing the polypeptide or derivative of this invention, comprising culturing a host cell comprising a recombinant nucleic acid encoding the polypeptide derivative operably linked to a promoter under conditions such that the polypeptide or derivative is produced by said host cell; and purifying the polypeptide or derivative.

In one aspect, the invention relates to a pharmaceutical composition comprising a therapeutically effective amount of the polypeptide described herein, the polypeptide derivative described herein, the nucleic acid described herein, the vector described herein, or the host cell described herein; and optionally further comprising a pharmaceutically acceptable carrier. In another aspect, the invention relates to a kit comprising in one or more containers the pharmaceutical composition described herein.

In one aspect, the invention relates to a method for inhibiting Notch activity in a subject, comprising administering to said subject the polypeptides or derivative of the invention. In another aspect, the invention relates to a method for inhibiting Notch activity in a subject, comprising administering to said subject a polypeptide comprising the DSL domain, the DOS domain, and the ELR4-6 domains of a Serrate protein. In one aspect, the invention relates to a method for inhibiting Notch activity in a subject, comprising administering to said subject a polypeptide comprising the amino terminus through the DOS domain of a Serrate protein, and the ELR4-6 domains of said Serrate protein. In a specific embodiment, the polypeptide lacks the transmembrane domain and the intracellular domain, and optionally an ELR other than ELRs 1-2 and ELRs 4-6.

In one aspect, the invention relates to a method for treating a disease involving increased Notch expression or activity relative to normal cells in a subject in need thereof, comprising administering to said subject the polypeptide described herein, the polypeptide derivative described herein, the nucleic acid described herein, the vector described herein, the host cell described herein, or the pharmaceutical composition described herein. In another aspect, the invention relates to a method for treating a disease involving increased Notch expression or activity relative to normal cells in a subject in need thereof, comprising administering to said subject a polypeptide comprising the DSL domain, the DOS domain, and the ELR4-6 domains of a Serrate protein, or a derivative of said polypeptide wherein 1-5 conservative amino acid substitutions are present in the Serrate protein sequence of said polypeptide. In one embodiment, the polypeptide comprises the amino terminus through the DOS domain of a Serrate protein, and the ELR4-6 domains of said Serrate protein. In a specific embodiment, the polypeptide lacks the transmembrane domain and the intracellular domain, and optionally an ELR other than ELRs 1-2 and ELRs 4-6. In one embodiment, the disease described herein is a cancer.

In one aspect, the invention relates to an antibody or fragment thereof that specifically binds an epitope containing at least a portion of ELR4-6 of a Serrate protein. In one embodiment, the antibody or fragment thereof is monoclonal.

In one aspect, the invention relates to a method for increasing the activity of Notch in a subject, comprising administering to the subject the antibody or fragment thereof of the invention.

In another aspect, the invention relates to a method for treating a disease involving decreased Notch expression or activity relative to normal cells in a subject in need thereof, comprising administering to said subject the antibody or fragment thereof of the invention. In one embodiment, the disease described herein is a cancer.

Additional advantageous features and functionalities associated with the compositions and methods of the present invention will be apparent from the detailed description. The publications and other materials used herein to illuminate the background of the invention, and in particular cases, to provide additional details respecting the practice, are incorporated herein by reference in their entireties.

### 4. BRIEF DESCRIPTION OF THE FIGURES

FIGS. 1A-L show the nucleotide sequence of Drosophila Serrate (Ser) mRNA, complete coding sequence (GenBank Accession No.: M35759.1; SEQ ID NO: 1) and the amino acid sequence of Drosophila Serrate (Ser) (GenBank Accession No.: AAA28938.1; SEQ ID NO: 2). The “*” indicates the stop codon.

FIG. 1M shows the amino acid sequence and domain structure of Drosophila Serrate (Ser) (GenBank Accession No.: AAA28938.1; SEQ ID NO: 2). The amino acid sequence of each domain is shown in square brackets, and the domain name is listed under each sequence.

FIGS. 2A-K show the nucleotide sequence of human Serrate-1 mRNA, complete coding sequence (GenBank Accession No.: U73936.1; SEQ ID NO: 3) and the amino acid sequence of human Serrate-1 (GenBank Accession No.: AAC52020.1; SEQ ID NO: 4). The “*” indicates the stop codon.

FIG. 2L shows the amino acid sequence and domain structure of human Serrate-1 (GenBank Accession No.: AAC52020.1; SEQ ID NO: 4). The amino acid sequence of each domain is shown in square brackets, and the domain name is listed under each sequence.

FIGS. 3A-K show the nucleotide sequence of human Serrate-2 mRNA, complete coding sequence (GenBank Accession No.: AF003521.1; SEQ ID NO: 5) and the amino acid sequence of human Serrate-2 (GenBank Accession No.: AAB61285.1; SEQ ID NO: 6). The “*” indicates the stop codon.

FIG. 3L shows the amino acid sequence and domain structure of human Serrate-2 (GenBank Accession No.: AAB61285.1; SEQ ID NO: 6). The amino acid sequence of each domain is shown in square brackets, and the domain name is listed under each sequence.

FIG. 4 shows Drosophila Serrate (Ser) protein structure and deletions. The central structure of this cartoon depicts the predicted Ser protein. The extracellular N-terminal regions show the DSL domain (named for Delta, Ser and Lag-2), followed by the 14 ELRs (numbered 1-14). ELR 1 and 2 constitute the DOS domain (Komatsu et al., 2008, Plos Biol. 8:e196) and ELRs 4, 6 and 10 of Ser are interrupted with non-EGF-like sequences (ovals) (Fleming et al., 1990, Genes Dev. 12A:2188-2201; Thomas et al., 1991, Development 3:749-761). The transmembrane segment and the intracellular segment are also depicted. Regions deleted by the individual constructs are shown adjacent to the central figure. For the Ser^{sec} molecule, the N-terminal region through amino acid 1020 is produced. The approximate location of the terminal amino acid for this construct is shown by the arrow at amino acid 1020 (amino acid numbers given in Fleming et al., 1990, Genes Dev. 12A:2188-2201).

FIGS. 5A-O show the results of ectopic expression of Ser deletion constructs via Gal4^{Ser2}. Ser constructs were expressed in the wing via Gal4^{Ser2} and assayed in the adult. A. Expression pattern of Gal4^{Ser2} in the late third instar imaginal wing disc (dorsal to left and posterior to the bottom) is mostly in the dorsal compartment along the dorsal/ventral boundary in the same pattern as endogenous Ser expression. B. Wild type wing. C. Ectopic expression of wild type Ser produces wings with serrated margins and delta-like wing veins. D. SerDel7 expression also demonstrates wing nicks similar to wild type expression. E. SerDel6 expression generates normal wing margins but incomplete wing venation. F. SerhydroΔ6 expression appears similar to expression of wild type Ser with wing margin nicks and delta-like wing veins. SerDel5 (G) and SerDel4 expression (H) produce wings indistinguishable from SerDel6 expression (see E of this figure). I. Expression of SerDel3 produces weak wing nicking and vein deltas similar to wild type Ser expression. J. Bright field of third instar wing imaginal disc. K. Expression of the NIRtom does not demonstrate any alteration of wild type wing morphology. L. Expression of Ser^{sec} demonstrates dominant-negativity as reduced wing size and exaggerated wing venation. (photo taken at same magnification as B of this figure). M. Expression of the Nterm6tom Ser form generates dominant-negative wings indistinguishable from expression of Ser^{sec}. Removal of the 6^{th} ELR from the Ser^{sec} form (Ser^{sec}Del6; N of this figure) or removal of ELR4 from Nterm6tom, (Nterm6Del4; O of this figure) greatly reduces the dominant-negativity of these constructs.

FIGS. 6A-C show the binding competition between cis- and trans-expressed forms of Ser in S2 cell aggregation. Stable lines of EGFP-labeled Notch cells were added to stable lines of tomato tagged Ser-expressing cells, allowed to mix for 24 hours at room temperature and were assayed for the formation of interacting cellular aggregates (Fehon et al., 1990, Cell 3:523-534). The Notch expressing cells were transiently transfected with tomato-tagged Ser DNA at concentrations ranging between 0.0 μg and 2.0 μg prior to mixing with Ser expressing cells to induce cis-inhibition by co-expressed Ser with Notch. When performed with wild type Ser DNA (A of this figure), effective inhibition of aggregation occurs in a dose dependent manner when the Ser expressing cells (Ser labeled columns) are mixed with the transfected Notch cells. When Ser expressing cells are not mixed with the transfected Notch cells (S2R+), self-aggregate formation is minimal. When SerDel6 is comparably used for this assay (B of this figure), aggregation is not significantly inhibited by cis-expression. When the SerDel3 form that does not activate Notch is used in this assay (C of this figure), efficient inhibition of aggregation is observed between these cell types.

FIGS. 7A-B show the Cis/Trans interactions of Notch and Serrate. Interactions between the Notch receptor and the Ser ligand are illustrated. Notch is represented as a dimer given the existing data, but can be a monomer or a multimer. An unoccupied receptor is illustrated in the left of A of this figure. The demonstrated binding region of Notch, ELRs 11 and 12 and the Ax region, ELRs 24-29, are illustrated. A. Trans activation. When Ser binds to Notch in trans, the NIR region is not involved but the N-terminal DSL region is used (Cordle et al., 2008, Nat. Struct. Mol. Biol. 8:849-857). Trans Notch-Ser engagement leads to a critical signal producing cleavage by metalloproteases (P), leading to Notch activation (Kopan and Ilagan, 2009, Cell 2:216-233). B. Cis-Inhibition. When Ser and Notch are co-expressed on the same cell, Ser interacts with Notch using both the DSL and DOS regions that interact with Notch ELRs 11 and 12 (Cordle et al., 2008, Nat. Struct. Mol. Biol. 8:849-857) and the NIR region that may interact with the Ax region of Notch. This interaction stabilizes the Notch dimer in an inactive state leading to signal inhibition.

FIG. 8 shows sequence alignments of the NIR region of Ser with Ser family and Delta homologs. ELR4 (SEQ ID NO: 22), ELR5 (SEQ ID NO: 28) and ELR6 (SEQ ID NO: 34) of Ser were used to find the best alignments with Human Jagged 1 (Jagged-1-1), Chicken Serrate 1 (C-Serrate-1), Xenopus Jagged 1 (X-Jagged-1), Zebrafish Jagged 1 (Jagged-1a) and Drosophila Delta. To perform the alignment, non-EGF-like interruptions in ELR4 (arrow) and ELR6 (arrowhead) were removed as they are not conserved in non-Drosophilid species and, at least, the interruption in ELR6 is not responsible for cis-inhibition. Sequence removed from ELR4 is: AQVVRTSHGRSNMGRPVRRSSSMRSLDHLRPEGQALNGSSSSGLVLGSLGLGGGLAPD (SEQ ID NO: 7) and the sequence removed from ELR6 is: HSAGIAANALLTTTATAIIGSNLSSTALLAALTSAVASTSLAIG (SEQ ID NO: 8). Strong conservation of sequences is seen between the Serrate and Jagged homologs but significantly less conservation is observed between Serrate and Delta.

### 5. DETAILED DESCRIPTION

Provided herein are polypeptides that comprise Serrate protein sequences and inhibit Notch signaling pathway activity, termed herein Notch inhibitory region containing (“NIR”) polypeptides. As described in the examples herein, the inventors have discovered that a region of the Serrate protein comprising the DSL and DOS domains (and optionally the sequences amino terminal to the DSL domain) together with ELR4-6 inhibits the Notch signaling pathway (i.e. Notch signal transduction).

The terms “Serrate” and “Jagged” are used interchangeably in this application, with Serrate therefore intended to encompass both mammalian Jagged and non-mammalian forms of the protein/gene.

Accordingly, in one aspect, provided herein is an NIR polypeptide or derivative thereof comprising the DSL domain and the DOS domain (or alternatively the amino terminus including or excluding the signal peptide through the DOS domain), as well as the ELR4-6 domains of a Serrate protein, wherein said polypeptide or derivative lacks at least one of the following domains of said Serrate protein: ELR3, ELR7, ELR8, ELR9, ELR10, ELR11, or ELR12. The invention also relates to nucleic acids that encode the NIR polypeptides or derivatives. The present invention further relates to host cells that express an NIR polypeptide or derivative thereof. The invention further relates to a method of producing the NIR polypeptides or derivatives. Also provided are antibodies and fragments thereof to ELR4-6 of a Serrate protein. The uses of the foregoing products are also provided, including therapeutic methods and compositions and kits based on the polypeptides and derivatives, nucleic acids, host cells and antibodies and fragments described herein.

5.1 NIR Polypeptides

Serrate is a type I transmembrane protein with a functional extracellular domain, a transmembrane domain, and an intracellular domain. Serrate exhibits a modular arrangement in its extracellular domain comprising an amino terminal domain followed by the DSL (Delta, Serrate, and Lag2) domain and multiple tandemly arrayed epidermal growth factor (EGF)-like repeats (ELRs). The DOS (Delta and OSM-11-like proteins) domain contains the first two ELRs (ELR1 and ELR2).

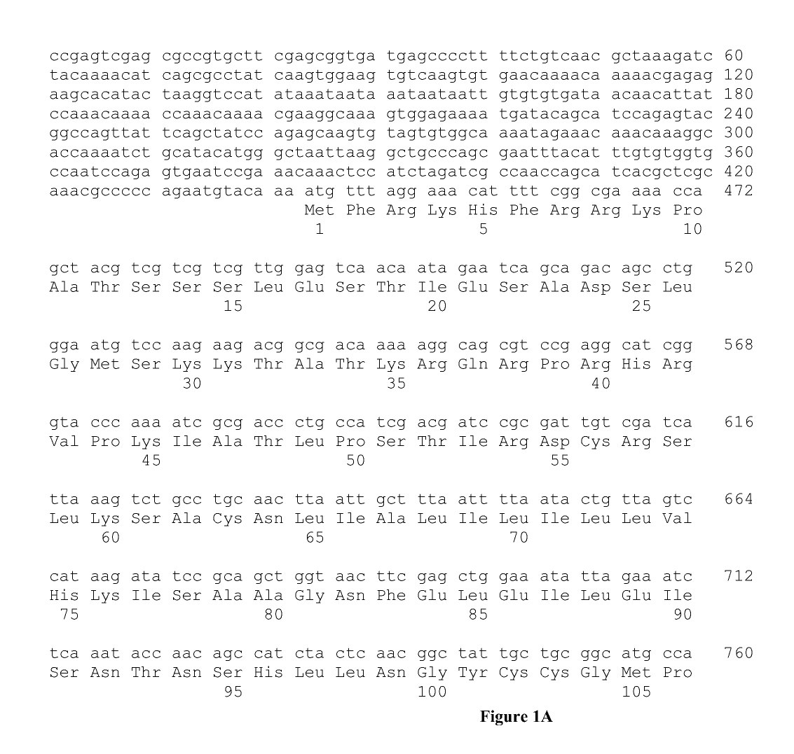

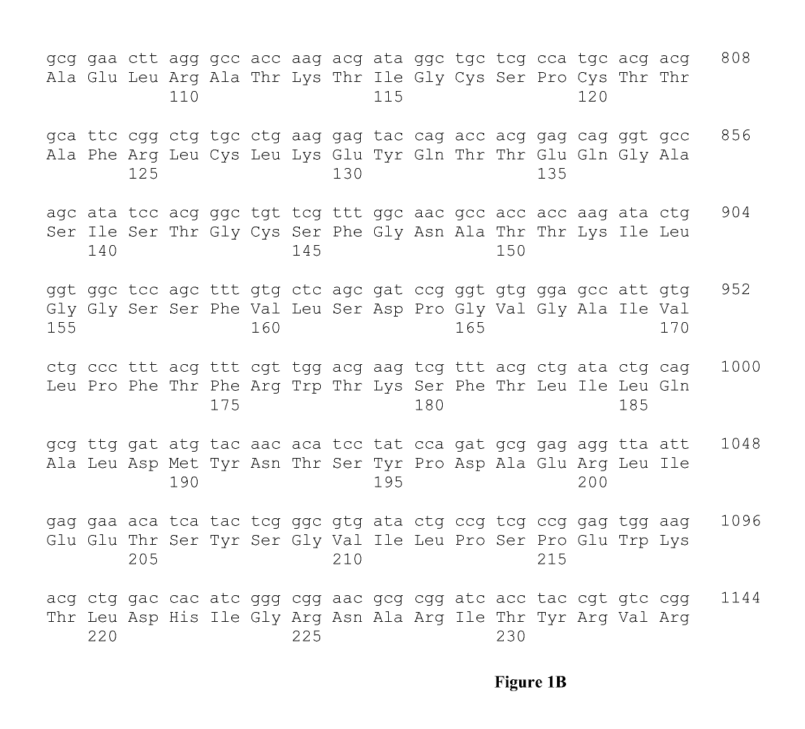

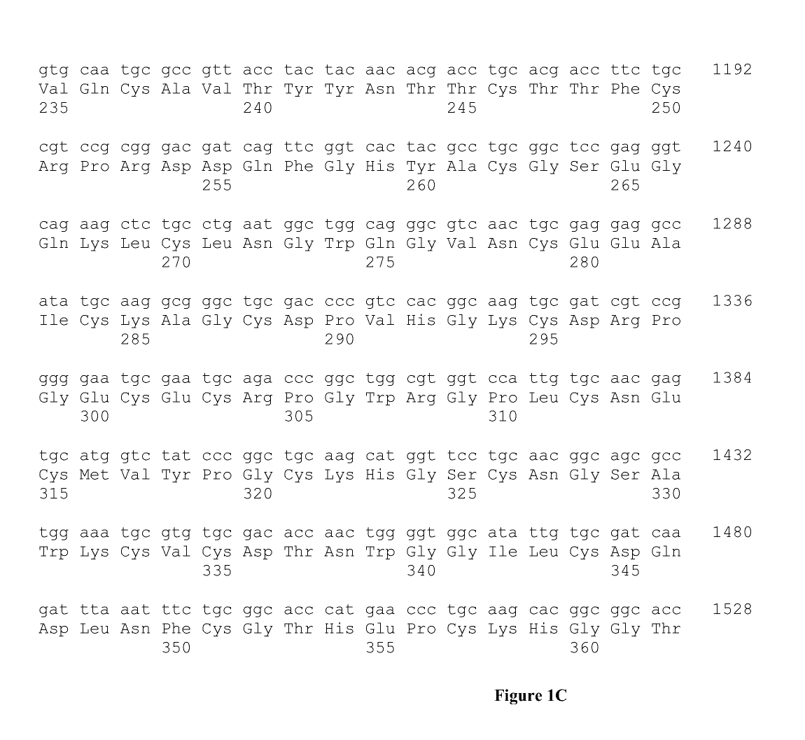

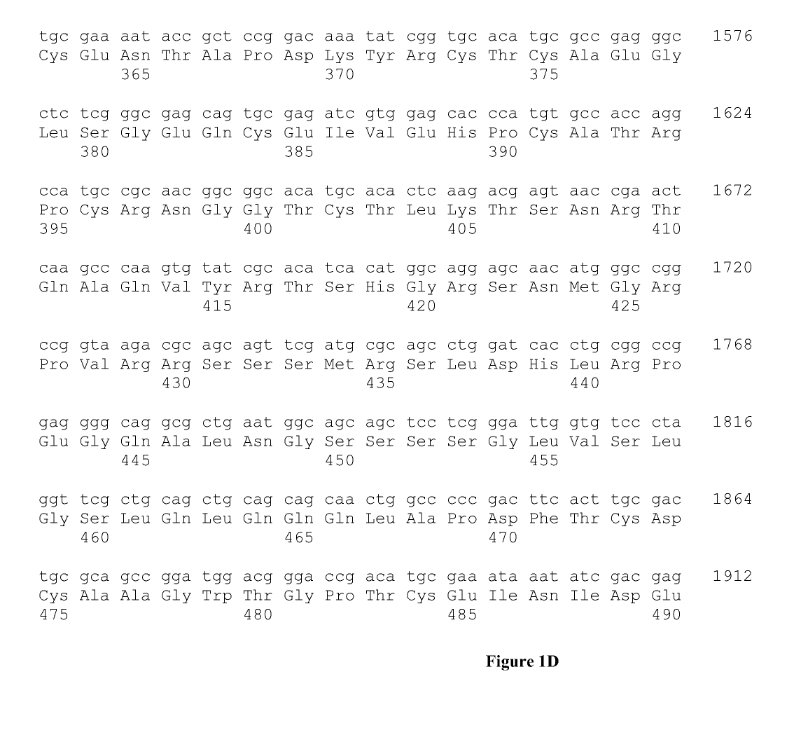

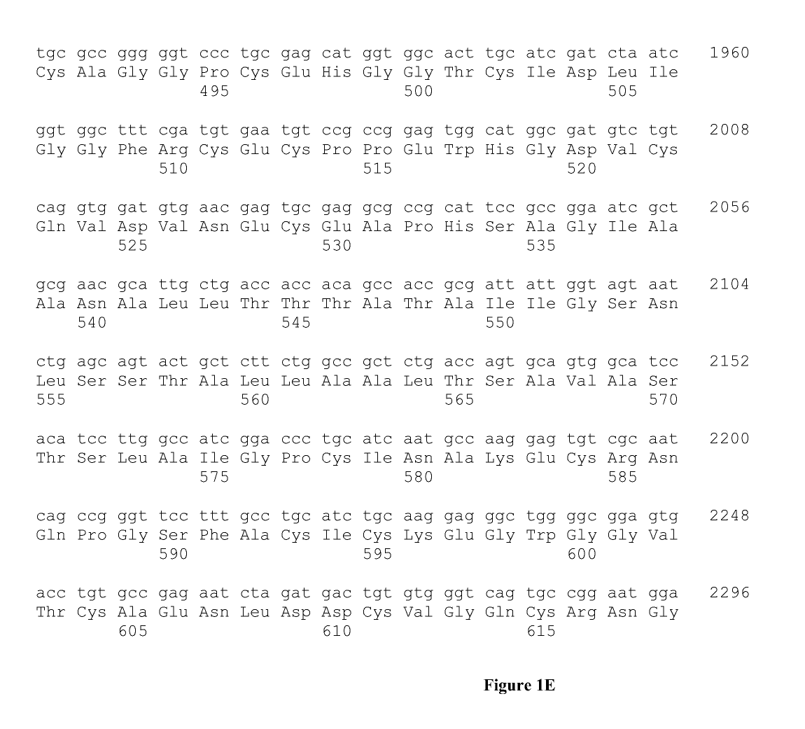

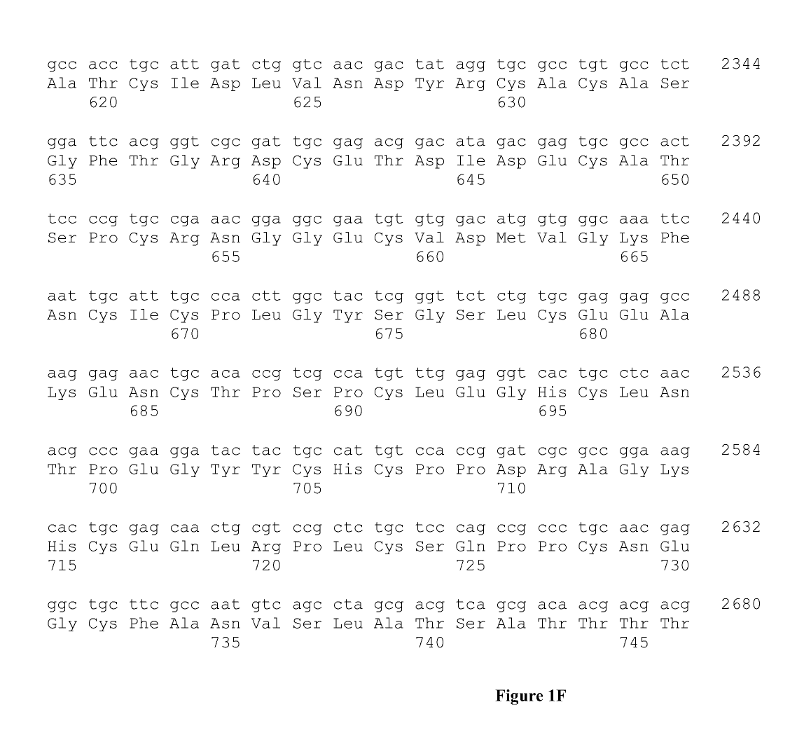

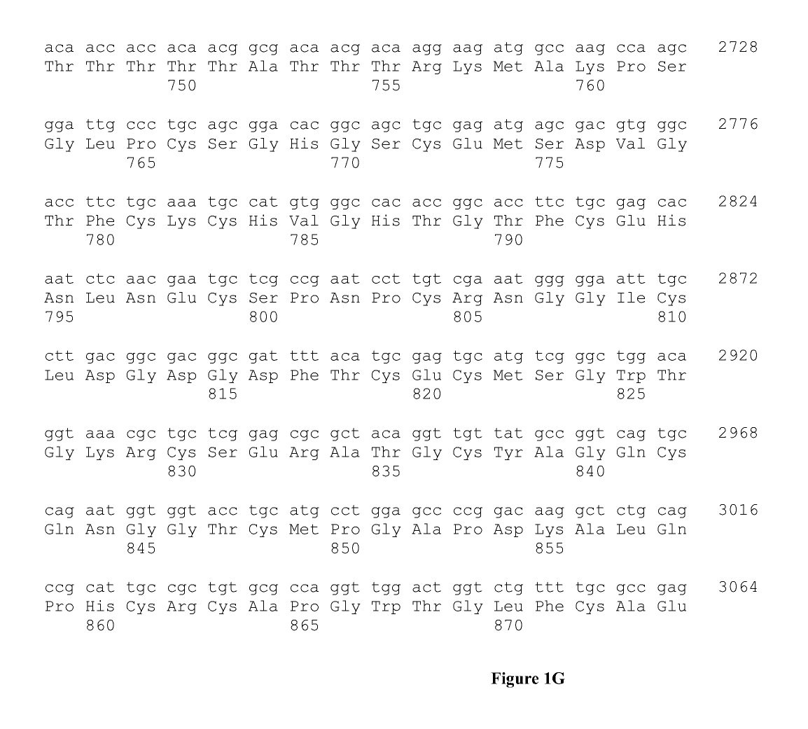

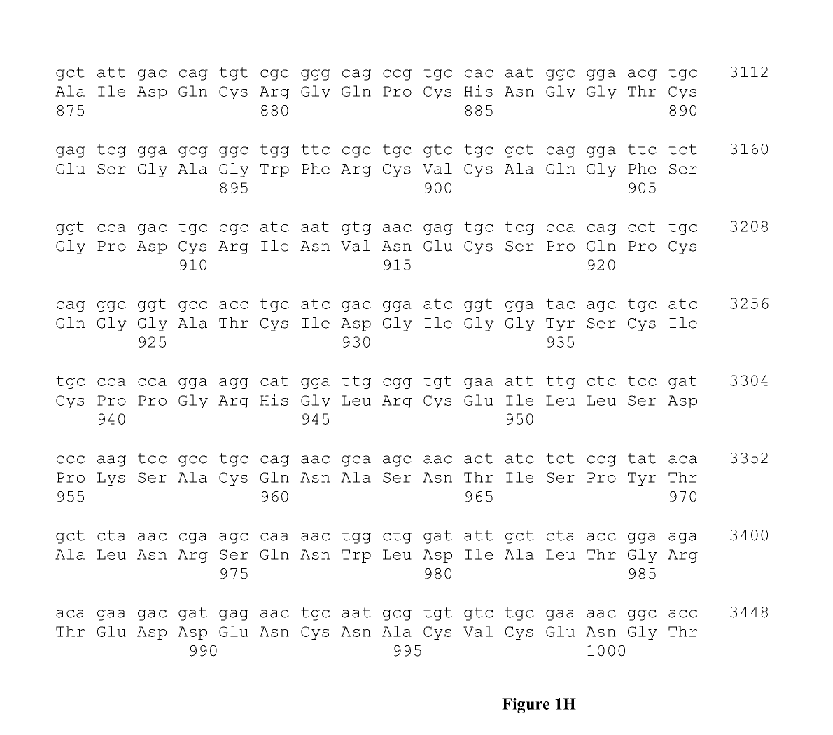

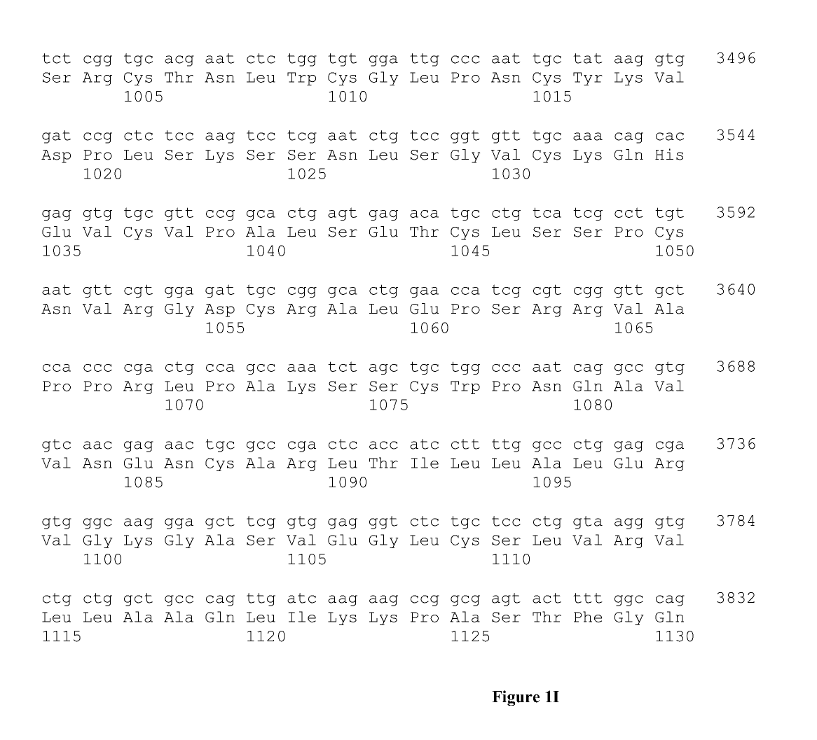

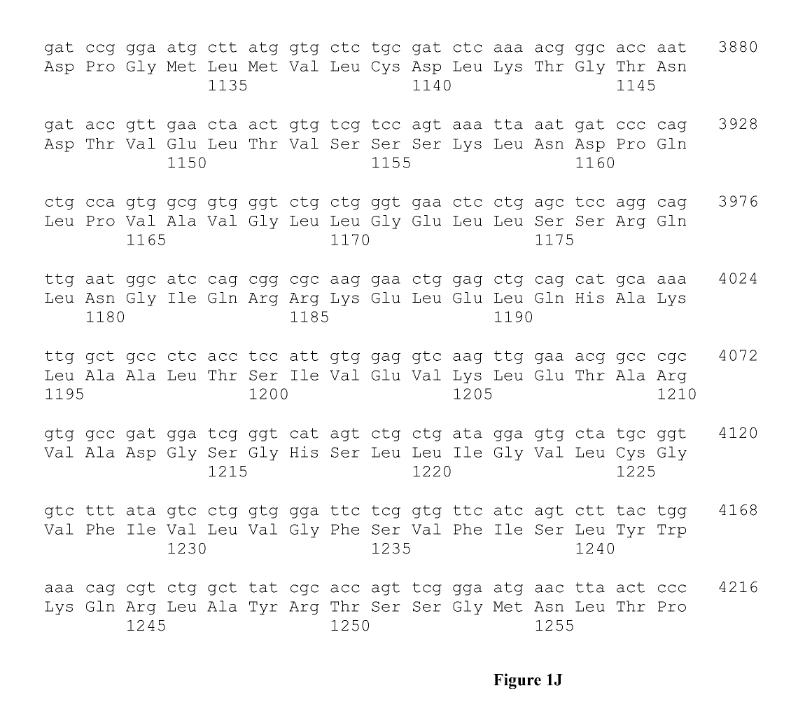

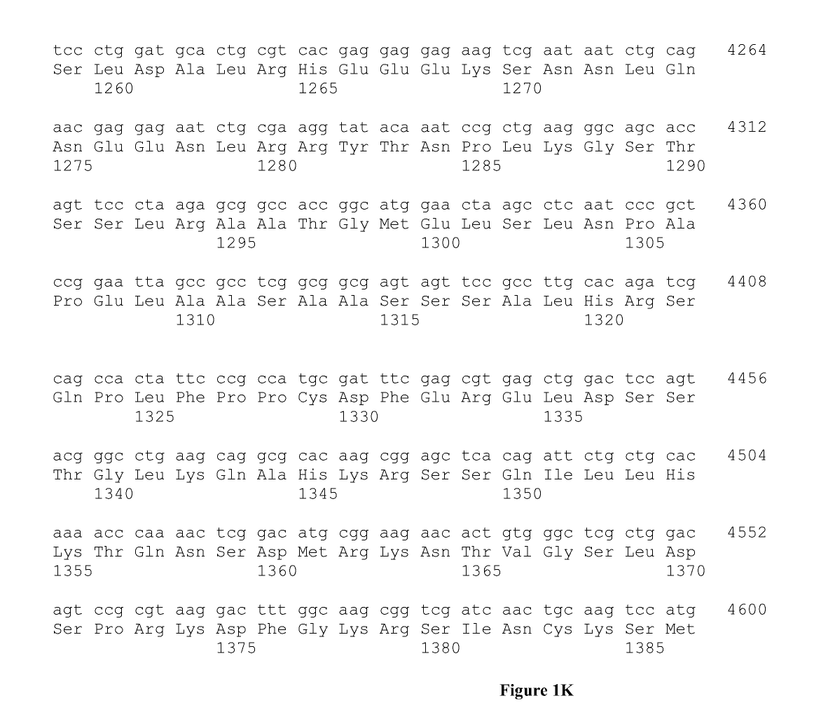

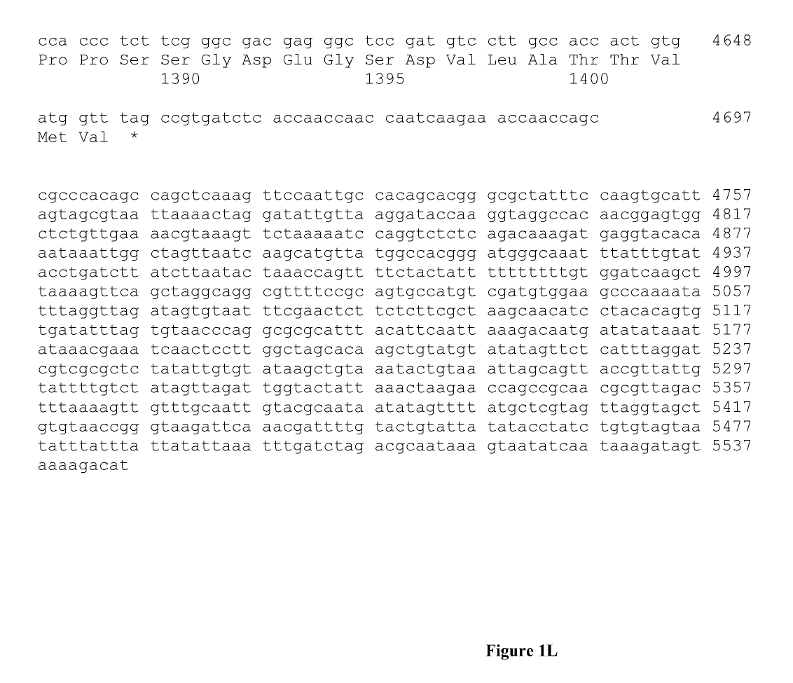

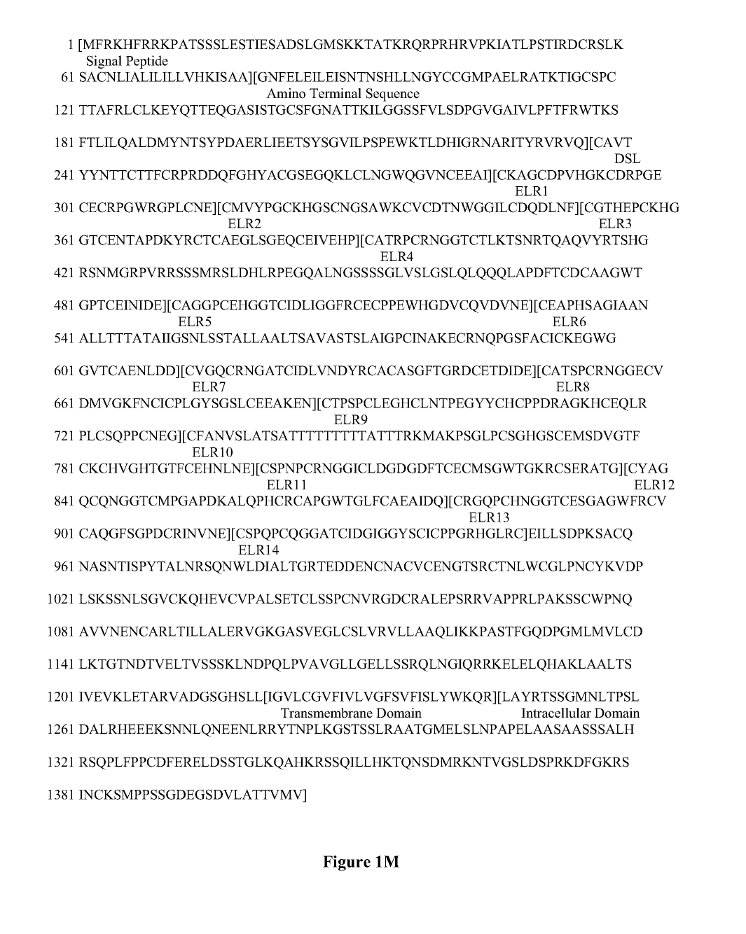

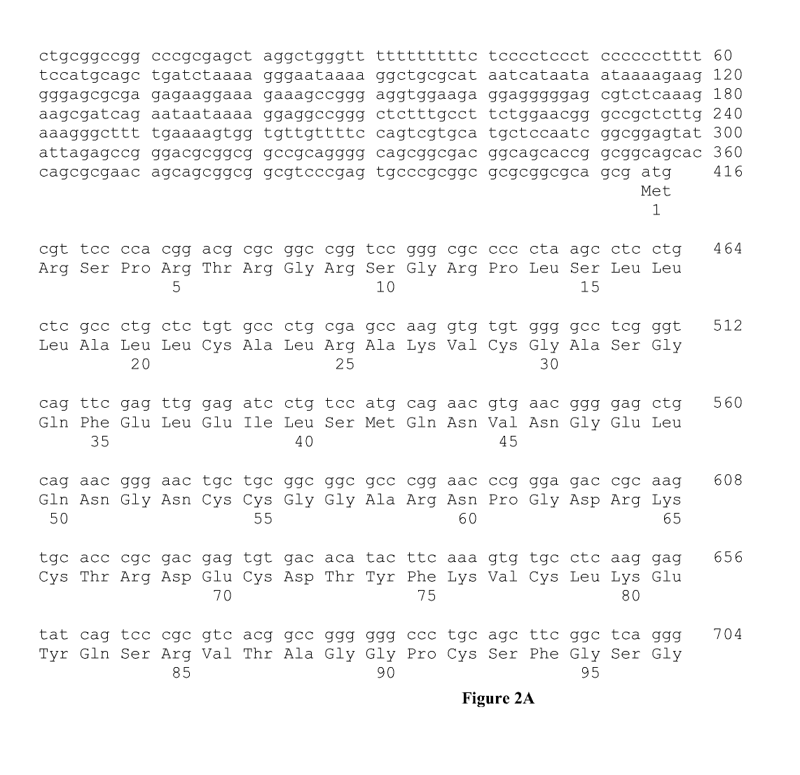

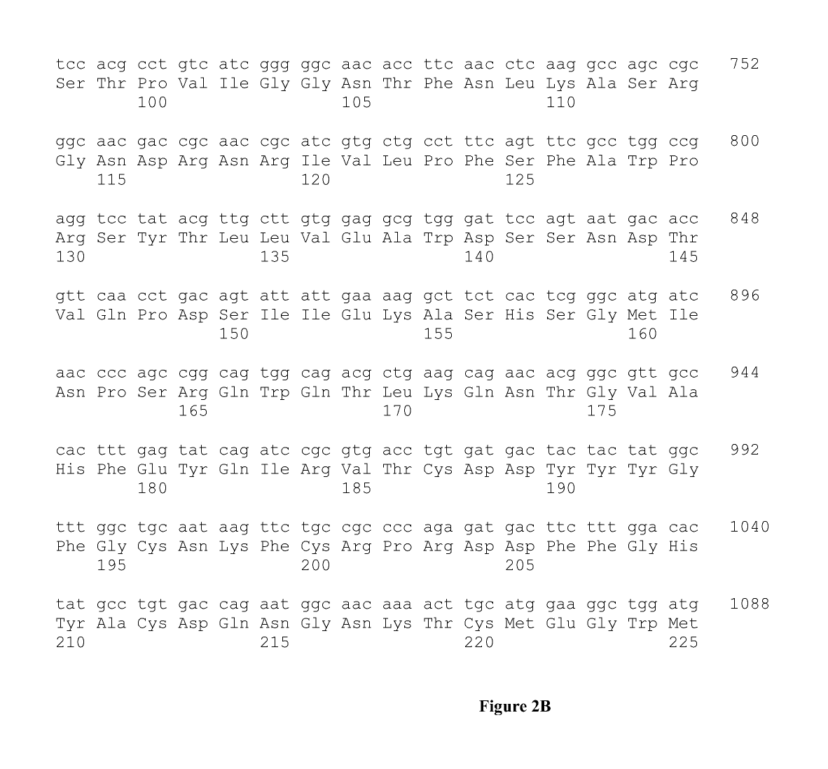

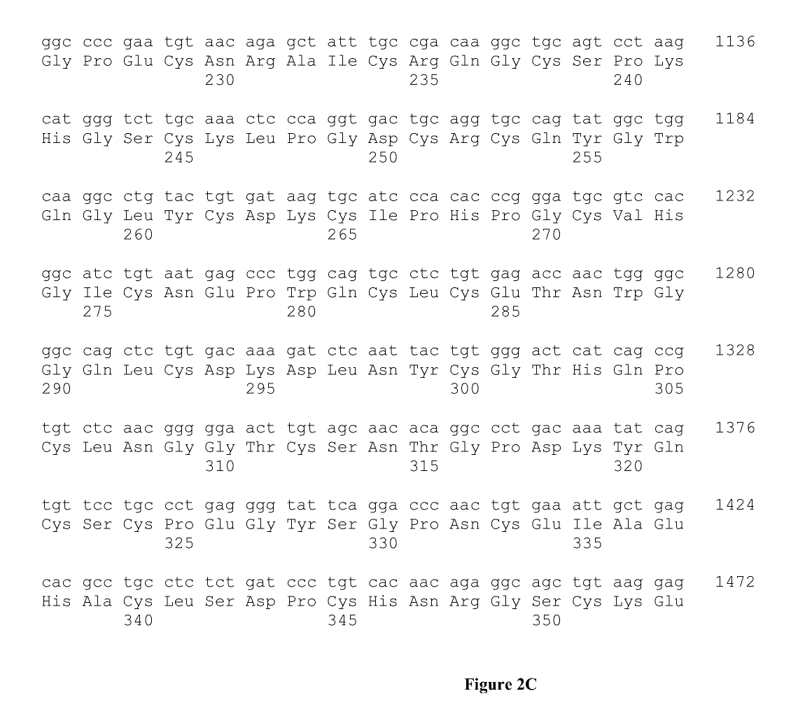

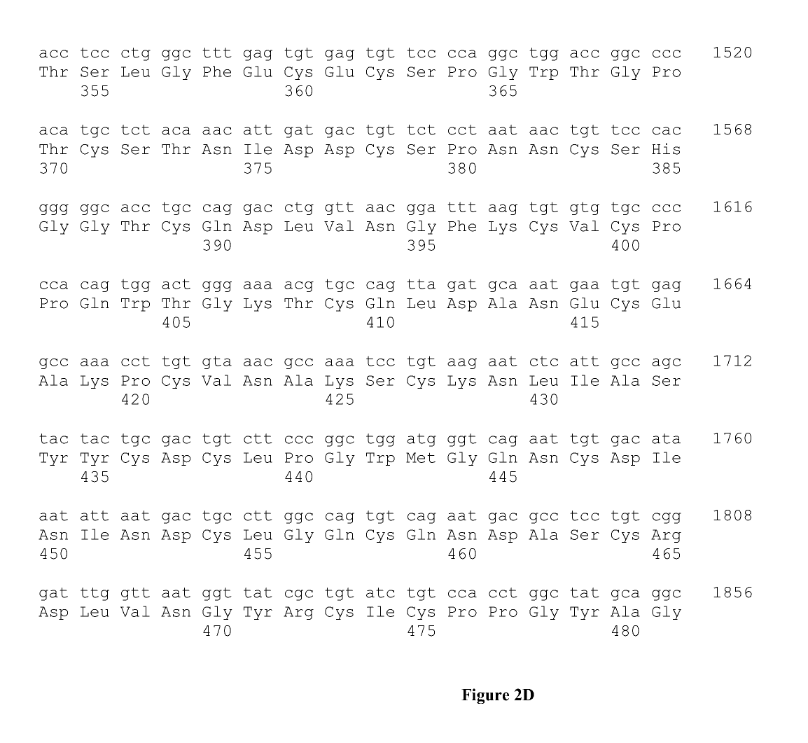

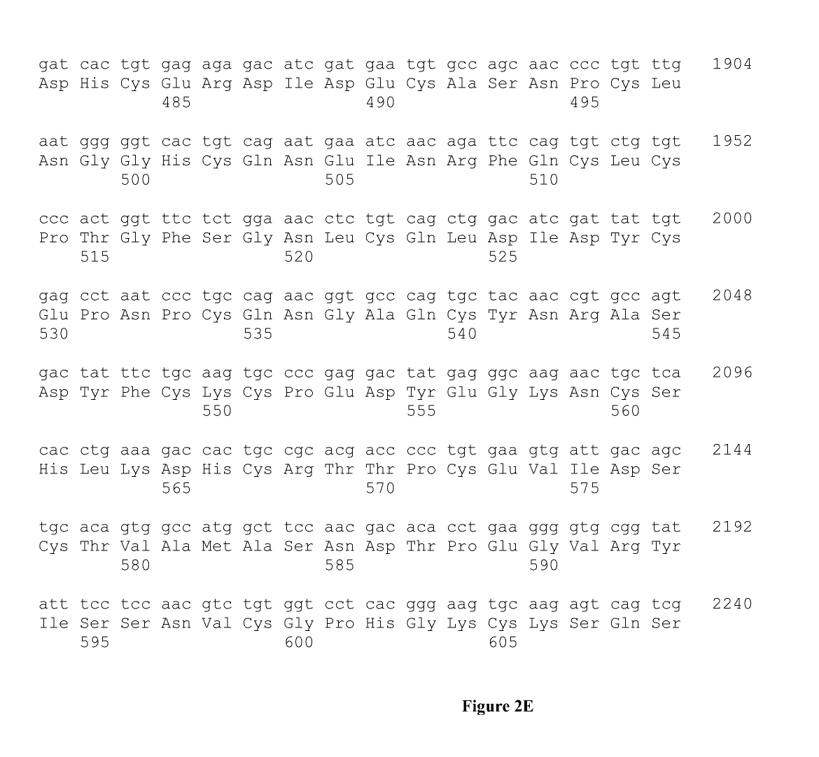

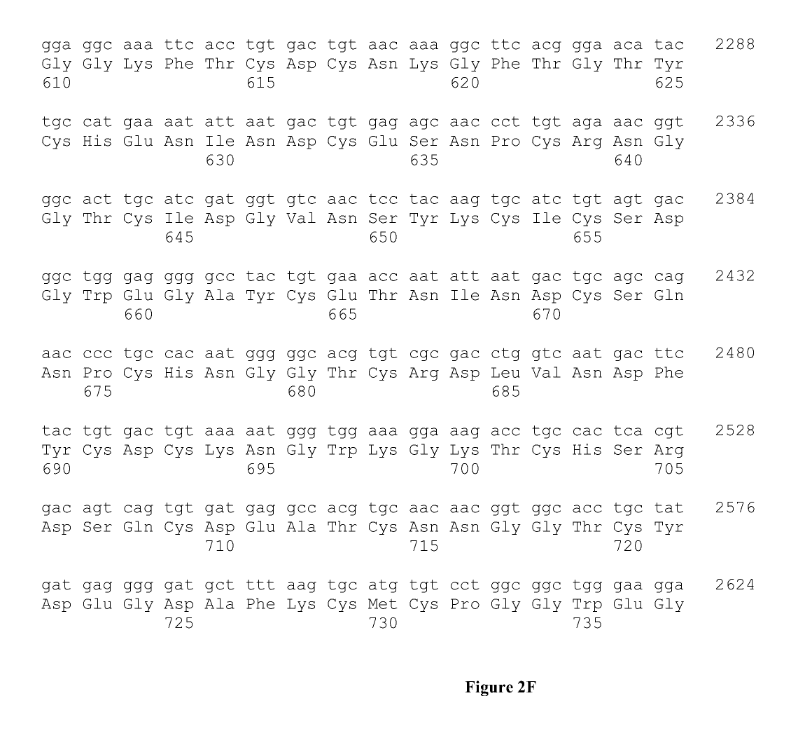

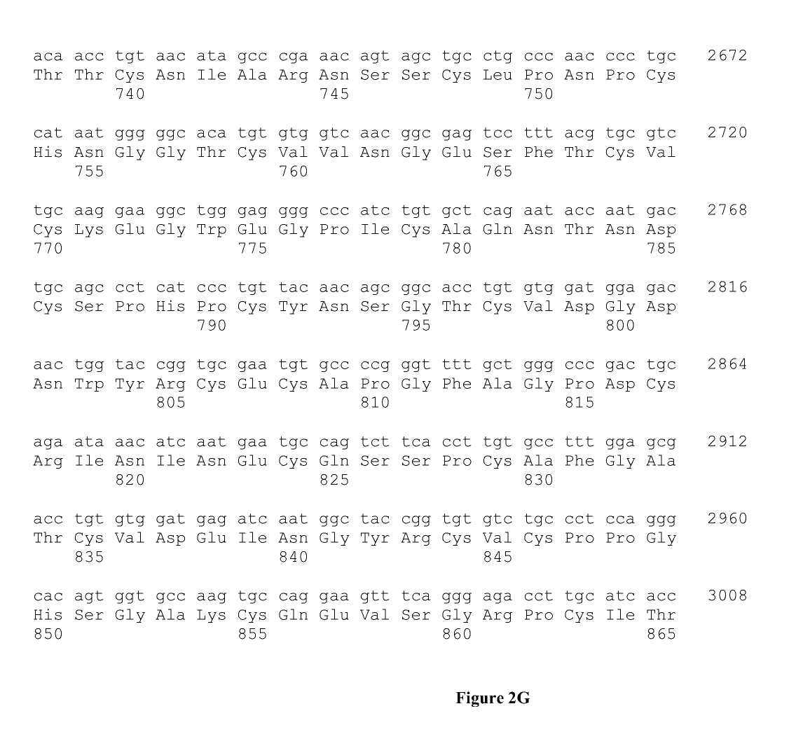

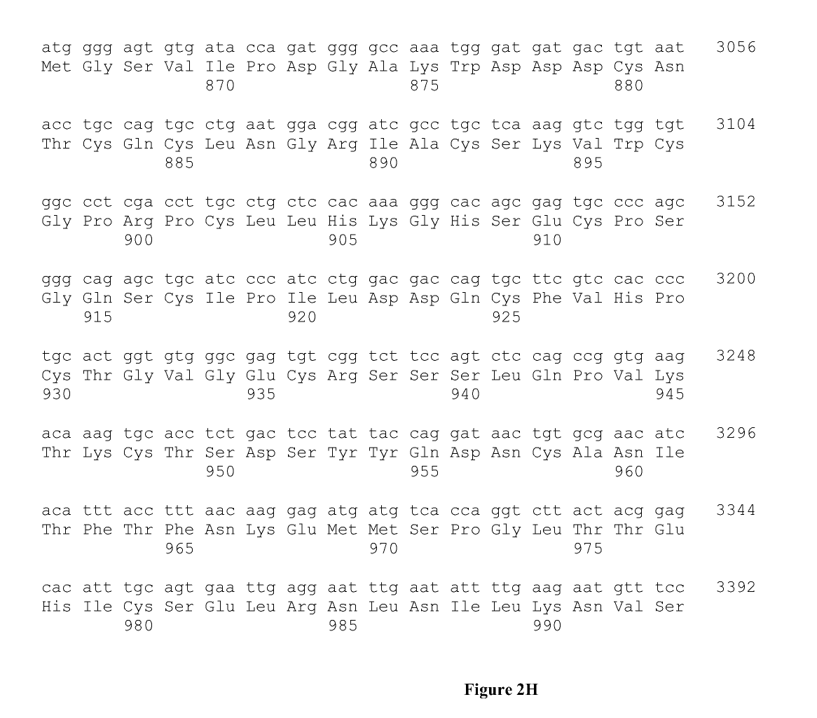

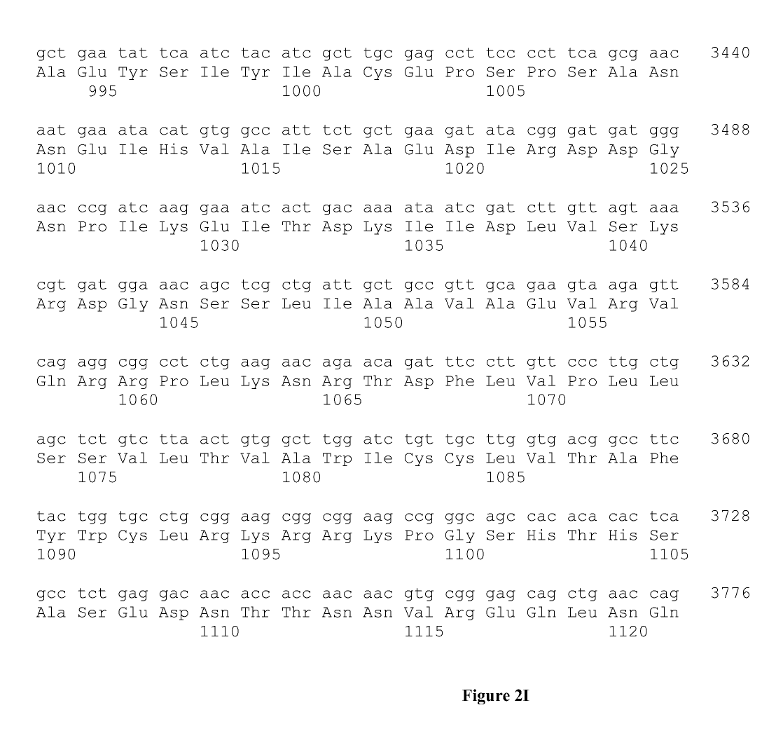

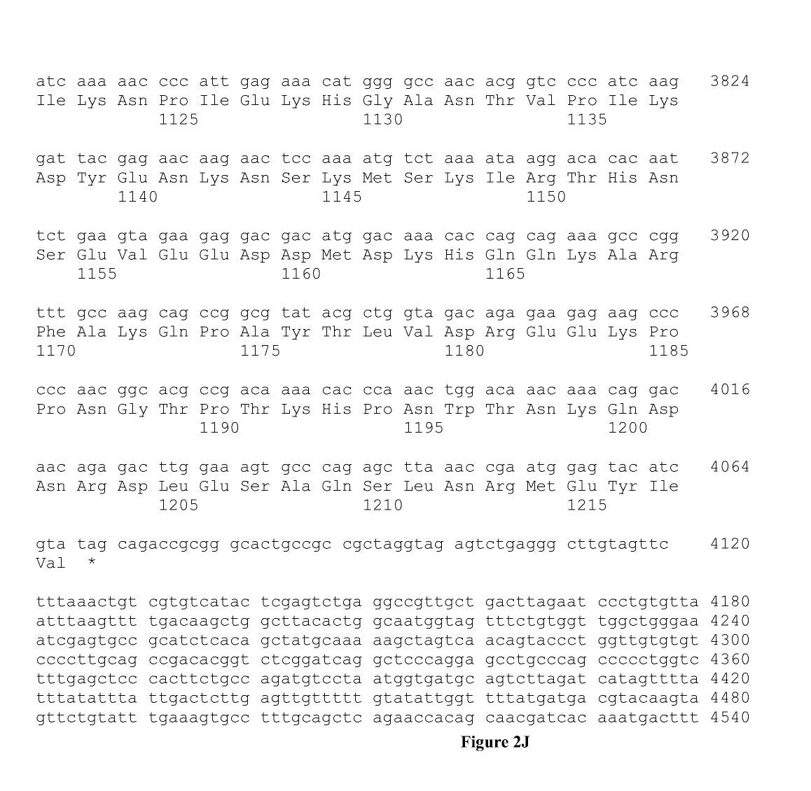

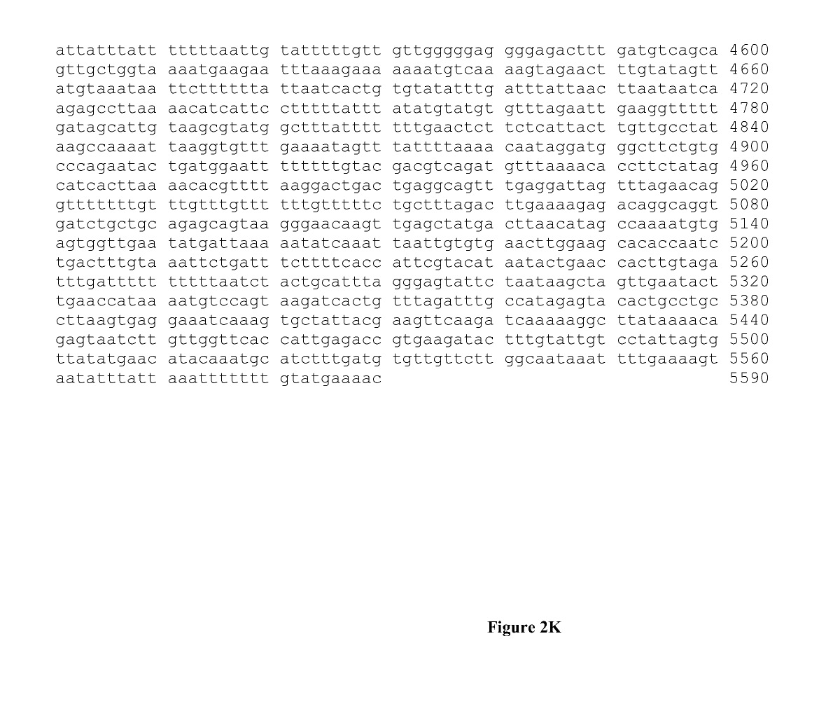

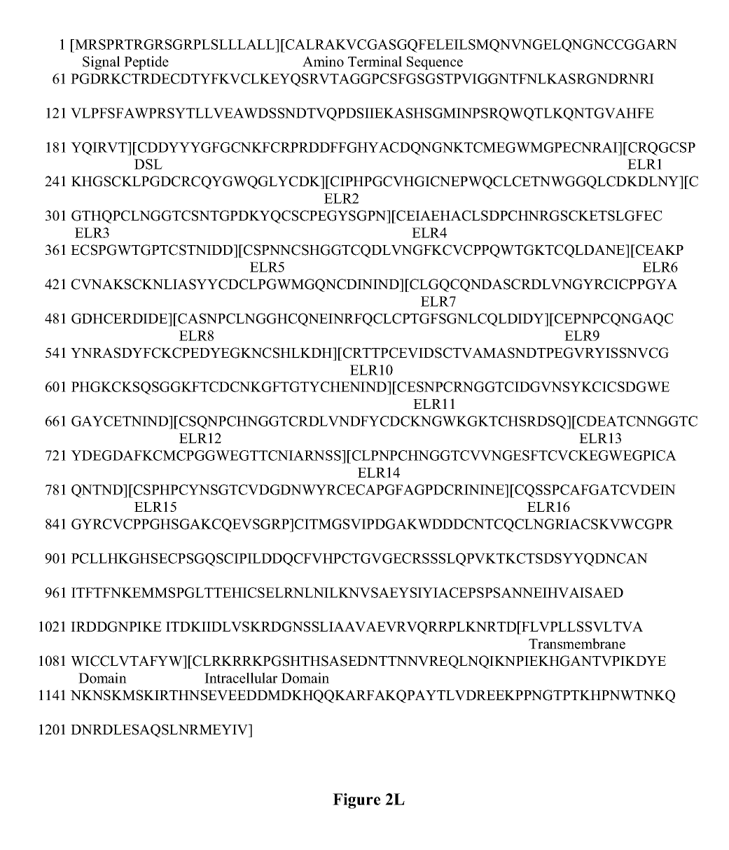

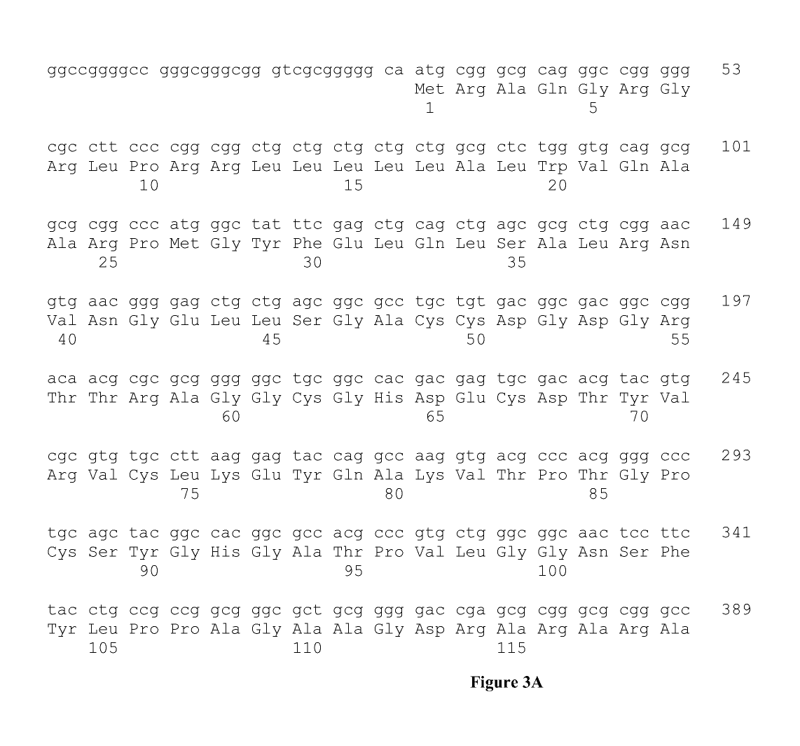

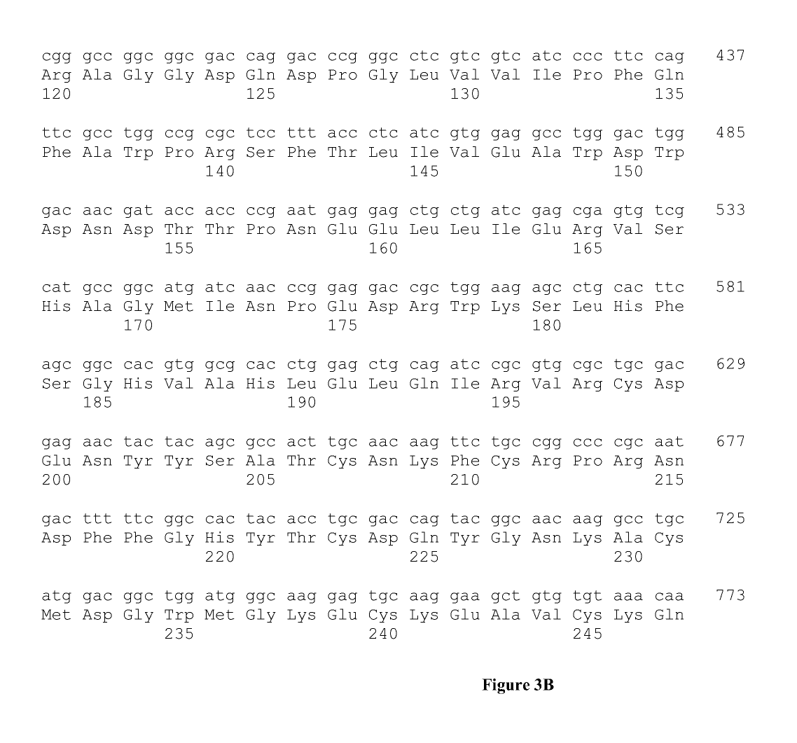

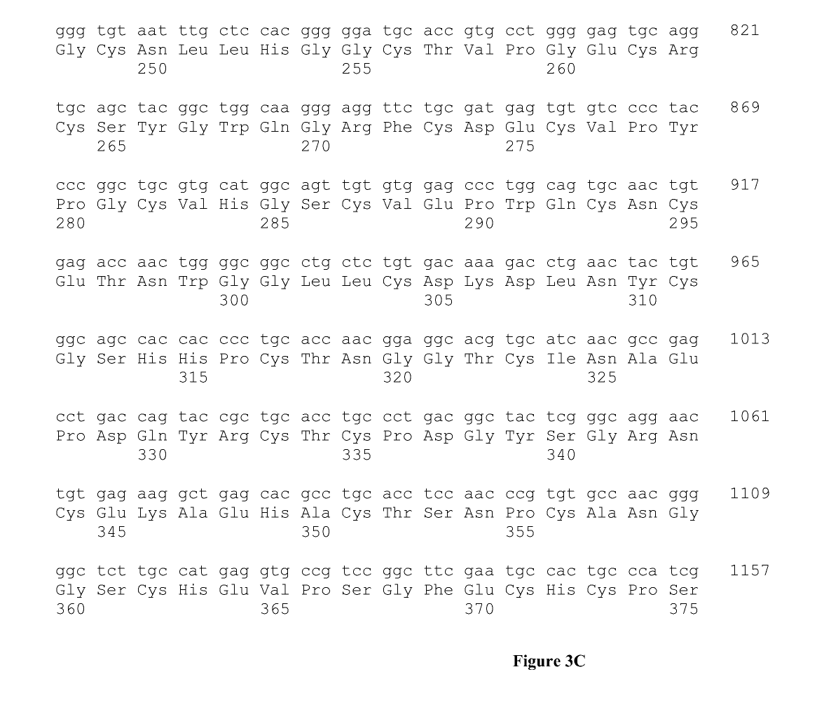

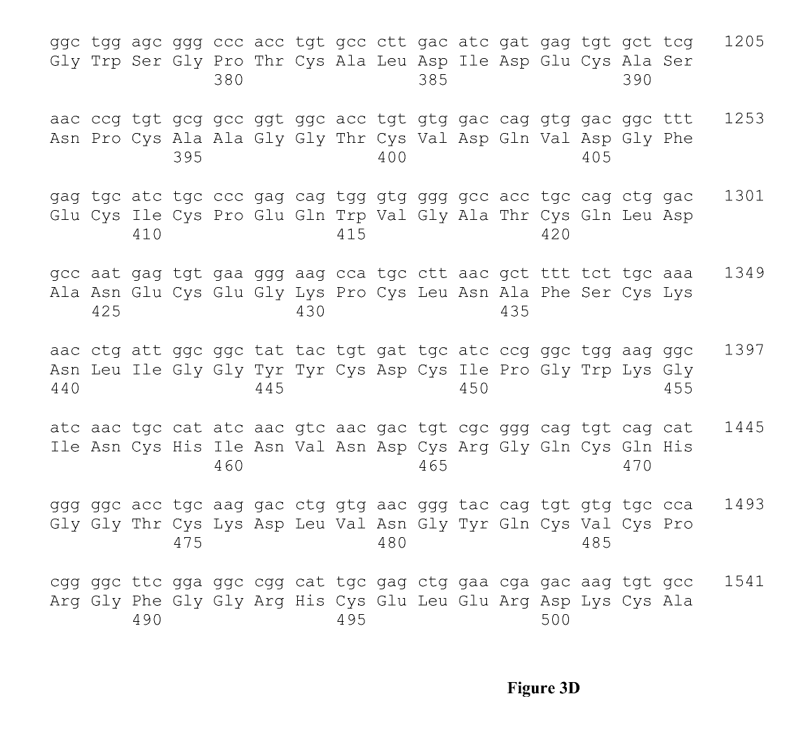

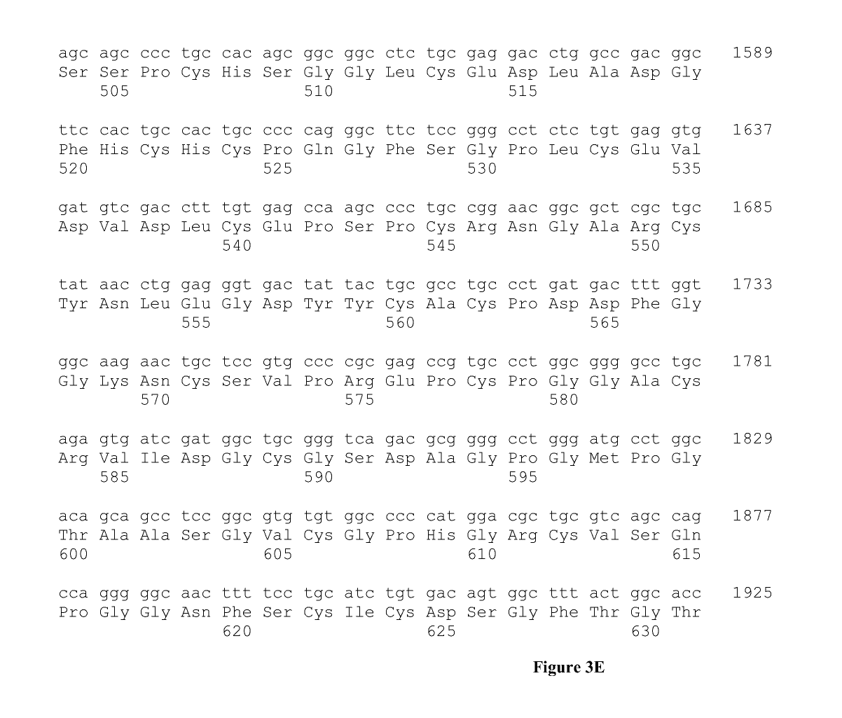

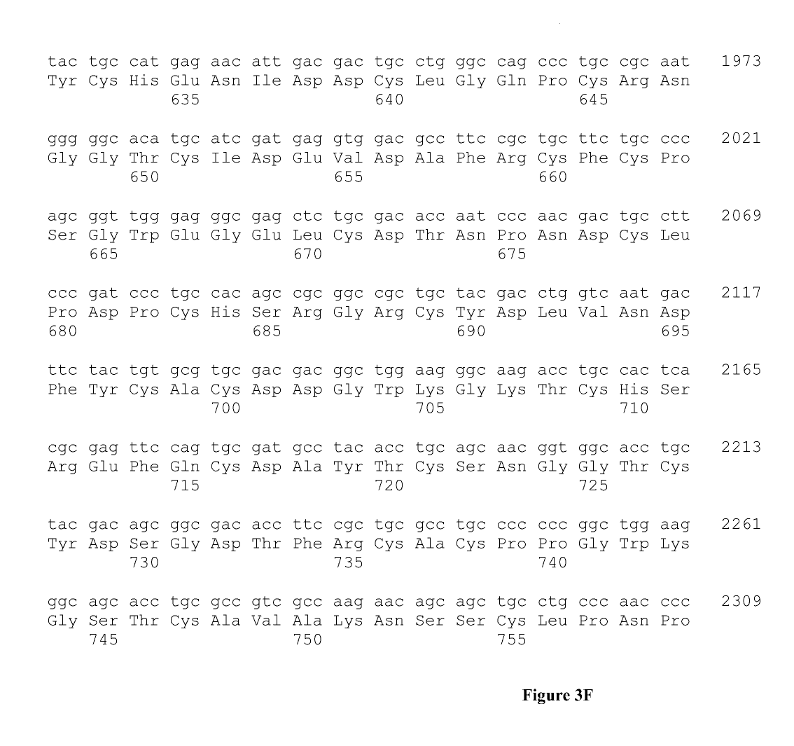

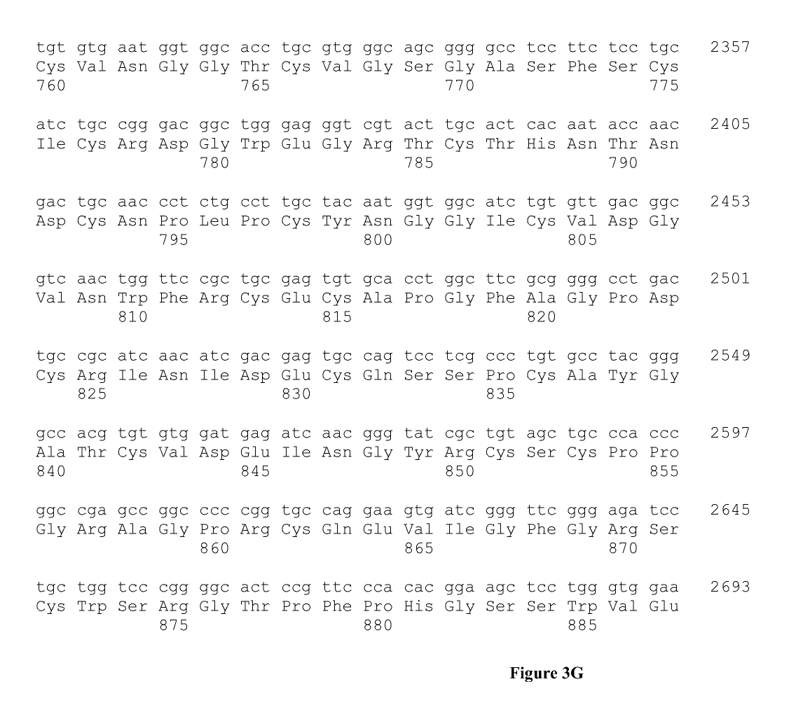

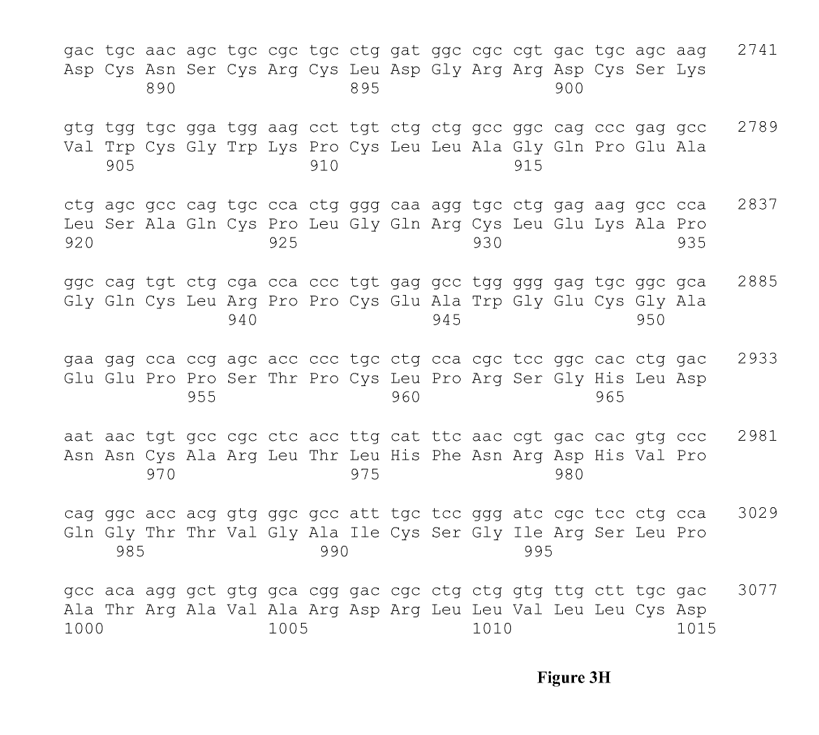

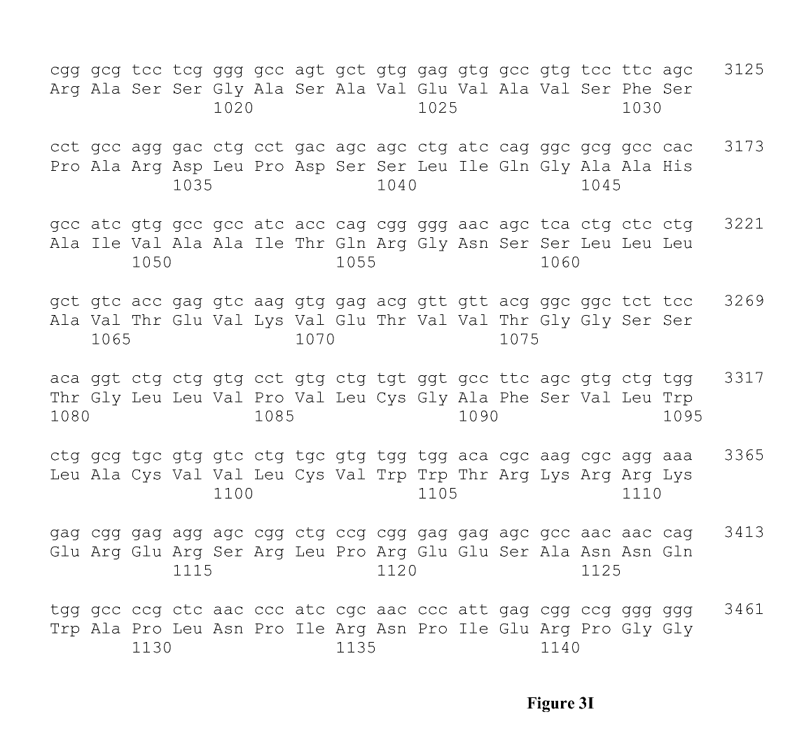

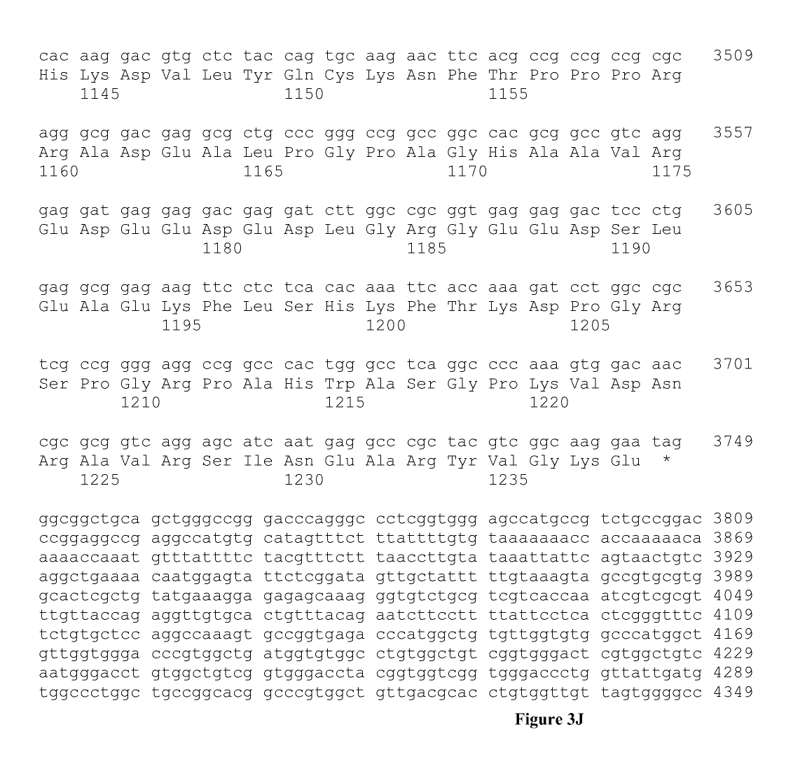

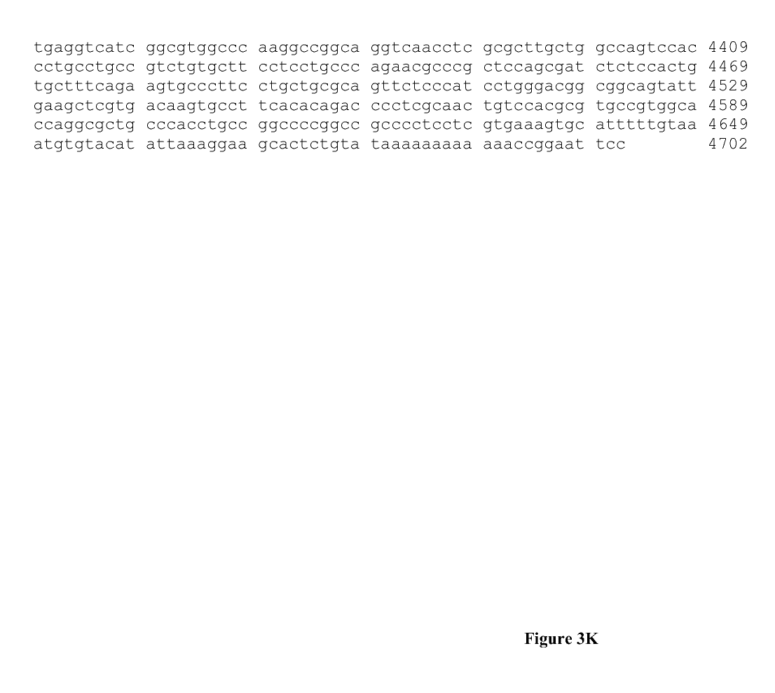

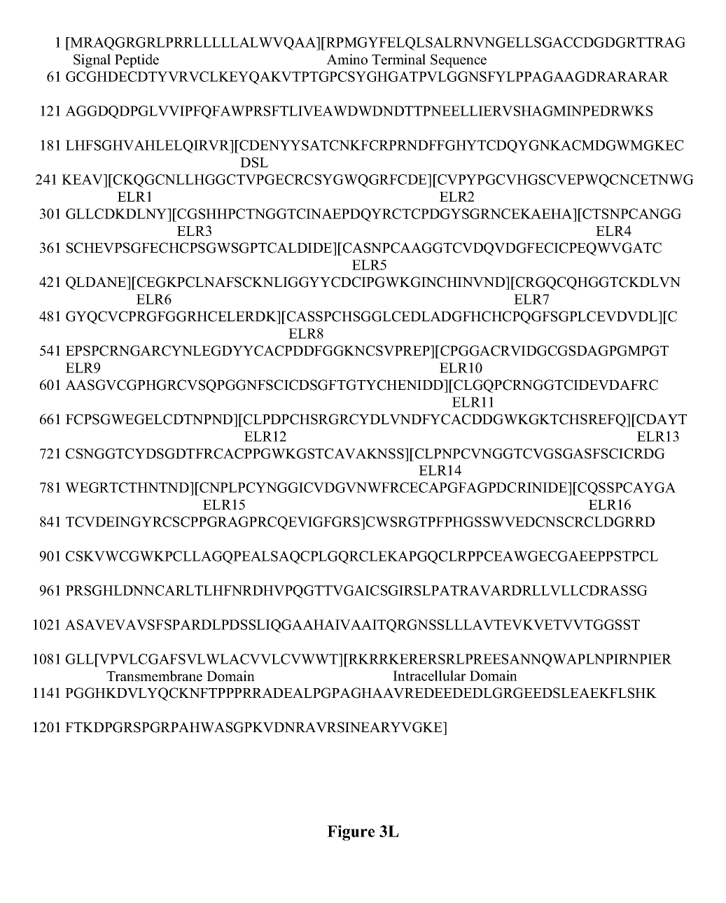

The amino acid sequences and domain structures of Drosophila Serrate (Ser), human Serrate-1 and human Serrate-2 are shown in FIGS. 1M, 2L and 3L, respectively.

From the amino terminus, the Drosophila Serrate (Ser) protein contains the signal peptide (amino acid numbers: 1-80) which can be cleaved and is thus believed not to be present in the mature protein, the amino terminal sequence (amino acid numbers: 81-236), the DSL domain (amino acid numbers: 237-283), ELR1 (amino acid numbers: 284-314), ELR2 (amino acid numbers: 315-350), ELR3 (amino acid numbers: 351-390), ELR4 (amino acid numbers: 391-490), ELR5 (amino acid numbers: 491-528), ELR6 (amino acid numbers: 529-610), ELR7 (amino acid numbers: 611-647), ELR8 (amino acid numbers: 648-685), ELR9 (amino acid numbers: 686-731), ELR10 (amino acid numbers: 732-798), ELR11 (amino acid numbers: 799-836), ELR12 (amino acid numbers: 837-878), ELR13 (amino acid numbers: 879-916), ELR14 (amino acid numbers: 917-948), transmembrane domain (amino acid numbers: 1221-1245) and intracellular domain (amino acid numbers: 1246-1404). All amino acid numbers in this paragraph are of SEQ ID NO: 2.

From the amino terminus, the human Serrate-1 protein contains the signal peptide (amino acid numbers: 1-21) which can be cleaved and is thus believed not to be present in the mature protein, the amino terminal sequence (amino acid numbers: 22-186), the DSL domain (amino acid numbers: 187-233), ELR1 (amino acid numbers: 234-264), ELR2 (amino acid numbers: 265-299), ELR3 (amino acid numbers: 300-338), ELR4 (amino acid numbers: 339-377), ELR5 (amino acid numbers: 378-415), ELR6 (amino acid numbers: 416-453), ELR7 (amino acid numbers: 454-490), ELR8 (amino acid numbers: 491-528), ELR9 (amino acid numbers: 529-566), ELR10 (amino acid numbers: 567-632), ELR11 (amino acid numbers: 633-670), ELR12 (amino acid numbers: 671-708), ELR13 (amino acid numbers: 709-747), ELR14 (amino acid numbers: 748-785), ELR15 (amino acid numbers: 786-823), ELR16 (amino acid numbers: 824-862), transmembrane domain (amino acid numbers: 1068-1091) and intracellular domain (amino acid numbers: 1092-1218). All amino acid numbers in this paragraph are of SEQ ID NO: 4.

From the amino terminus, the human Serrate-2 protein contains the signal peptide (amino acid numbers: 1-24) which can be cleaved and is thus believed not to be present in the mature protein, the amino terminal sequence (amino acid numbers: 25-197), the DSL domain (amino acid numbers: 198-244), ELR1 (amino acid numbers: 245-275), ELR2 (amino acid numbers: 276-310), ELR3 (amino acid numbers: 311-350), ELR4 (amino acid numbers: 351-388), ELR5 (amino acid numbers: 389-426), ELR6 (amino acid numbers: 427-464), ELR7 (amino acid numbers: 465-501), ELR8 (amino acid numbers: 502-539), ELR9 (amino acid numbers: 540-577), ELR10 (amino acid numbers: 578-639), ELR11 (amino acid numbers: 640-677), ELR12 (amino acid numbers: 678-715), ELR13 (amino acid numbers: 716-754), ELR14 (amino acid numbers: 755-792), ELR15 (amino acid numbers: 793-830), ELR16 (amino acid numbers: 831-871), transmembrane domain (amino acid numbers: 1084-1106) and intracellular domain (amino acid numbers: 1107-1238). All amino acid numbers in this paragraph are of SEQ ID No: 6.

In one embodiment, provided herein is an NIR polypeptide comprising the DSL domain, the DOS domain, and the ELR4-6 domains of a Serrate protein (e.g., human Serrate-1 or human Serrate-2), wherein said polypeptide lacks at least one of the following domains of said Serrate protein: ELR3, ELR7, ELR8, ELR9, ELR10, ELR11, or ELR12. In one embodiment, provided herein is an NIR polypeptide comprising the amino terminus through the DOS domain of a Serrate protein, and the ELR4-6 domains of said Serrate protein, wherein said polypeptide lacks at least one of the following domains of said Serrate protein: ELR3, ELR7, ELR8, ELR9, ELR10, ELR11, or ELR12. In one embodiment, the NIR polypeptide lacks the ELR3 domain of said Serrate protein. In a specific embodiment, the NIR polypeptide lacks each of the ELR3, ELR7, ELR8, ELR9, ELR10, ELR11, and ELR12 domains of said Serrate protein. In another embodiment, the NIR polypeptide lacks at least one of the following domains of said Serrate protein: ELR7, ELR8, ELR9, ELR10, ELR11, or ELR12. In a specific embodiment, the NIR polypeptide lacks each of the ELR7, ELR8, ELR9, ELR10, ELR11, and ELR12 domains of said Serrate protein. In one embodiment, the NIR polypeptide lacks all ELRs except ELR4-6 of said Serrate protein. In another embodiment, the NIR polypeptide lacks all ELRs except ELR3-6 of said Serrate protein. In one embodiment, the NIR polypeptide further comprises ELR3 of said Serrate protein. In another embodiment, the NIR polypeptide comprises a fragment of said Serrate protein that spans the amino terminus through ELR6, ELR7, ELR8, ELR9, ELR10 or ELR11. In one embodiment, the NIR polypeptide lacks the transmembrane domain and the intracellular domain of said Serrate protein.

The invention also provides an NIR polypeptide consisting of the DSL domain, the DOS domain, and the ELR4-6 domains of a Serrate protein. In another embodiment, an NIR polypeptide consists of the DSL domain, the DOS domain, and the ELR3-6 domains of a Serrate protein.

The NIR polypeptide can further comprise a non-Serrate protein amino acid sequence. The non-Serrate protein amino acid sequence can be a fusion protein sequence (e.g., His-tag, FLAG-Tag, GST, GFP, an antibody fragment, e.g., an antibody Fc region, signal peptides, sequences for selection purposes, etc.). In a specific embodiment, the non-Serrate protein amino acid sequence is of an immunoglobulin Fc (IgFc) domain.

The Serrate protein can be a mammalian Serrate protein. In a specific embodiment, the mammalian Serrate protein is a human Serrate protein (e.g., human Serrate-1 or human Serrate-2). In a specific embodiment, the human Serrate protein is human Serrate-1. In another embodiment, the human Serrate protein is human Serrate-2.

Also provided herein are NIR polypeptide derivatives. Preferably the derivatives retain function, as judged by any one or more criteria, including but not limited to the resulting biological effect, e.g., inhibition of Notch signal transduction. In one embodiment, amino acid residue substitutions, additions or deletions within the Serrate amino acid sequence of the NIR polypeptide result in a silent or conservative change and produce a functional product. In one embodiment, one amino acid addition to the Serrate amino acid sequence of the NIR polypeptide is made. In one embodiment, one amino acid deletion in the Serrate amino acid sequence of the NIR polypeptide is made. In one embodiment, one conservative amino acid substitution in the Serrate amino acid sequence of the NIR polypeptide is made. In one embodiment, one or more, preferably 1-5, conservative substitutions in the Serrate amino acid sequence of the NIR polypeptide is made. In one embodiment, a conservative amino acid substitution in the Serrate amino acid sequence of the NIR polypeptide is one in which the amino acid residue is replaced with an amino acid residue having a side chain with a similar charge. Families of amino acid residues having side chains with similar charges have been defined in the art. These families include amino acids with nonpolar side chains (e.g., alanine, valine, leucine, isoleucine, proline, phenylalanine, methionine, tryptophan), uncharged polar side chains (e.g., glycine, asparagine, glutamine, serine, threonine, tyrosine, cysteine), basic side chains (e.g., lysine, arginine, histidine), acidic side chains (e.g., aspartic acid, glutamic acid), beta-branched side chains (e.g., threonine, valine, isoleucine) and aromatic side chains (e.g., tyrosine, phenylalanine, tryptophan, histidine). In one embodiment, the NIR polypeptide has one, two, or up to five additions, deletions or conservative amino acid substitutions in the Serrate amino acid sequence contained therein. In one embodiment, all or part of the coding sequence of the NIR polypeptide is subjected to random mutagenesis (e.g., saturation mutagenesis), and the resultant mutants are screened to identify NIR polypeptide derivatives that retain function as described above.

In a specific embodiment, the NIR polypeptide or derivative is isolated or purified. As used herein, the terms “purified” and “isolated” when used in the context of a polypeptide or derivative that is obtained from a recombinant cell or organism, refers to a polypeptide or derivative, respectively, which is substantially free of contaminating materials, e.g., cell culture medium, and cellular materials from the source, such as but not limited to cell wall materials, cell debris, organelles, membranes, the bulk of the proteins, nucleic acids, carbohydrates, and/or lipids present in cells. In one embodiment, more than about 70%, 75%, 80%, 85%, 90%, 95%, 98% or 99% of total protein weight in the NIR polypeptide or derivative preparation obtained from a recombinant cell or organism is the NIR polypeptide or derivative, respectively. As used herein, the terms “purified” and “isolated” when used in the context of a polypeptide or derivative that is produced by chemical synthesis, refers to a polypeptide or derivative, respectively, which is substantially free of chemical precursors or other chemicals which are involved in the synthesis of the polypeptide or derivative. In one embodiment, more than about 70%, 75%, 80%, 85%, 90%, 95%, 98% or 99% of total protein weight in the NIR polypeptide or derivative preparation produced by chemical synthesis is the NIR polypeptide or derivative, respectively.

5.2 Nucleic Acids Encoding NIR Polypeptides and Recombinant Expression

Provided herein also are nucleic acids comprising nucleotide sequences encoding NIR polypeptides or derivatives thereof (termed herein “NIR nucleic acid”), and vectors, e.g., vectors comprising nucleotide sequences of the NIR nucleic acids for recombinant expression in host cells. In certain embodiments, the NIR nucleic acid is a DNA (e.g., a cDNA) or RNA. In certain embodiments, the NIR nucleic acid is double stranded or single stranded. In any of the above embodiments, the NIR nucleic acid can include conservative variations. In one embodiment, such conservative variations are degenerate codon substitutions. In any of the above embodiments, the NIR nucleic acid can include complementary sequences.

In one embodiment, the NIR nucleic acid is purified. In one embodiment, the purified NIR nucleic acid is a cDNA. In specific embodiments, the purified NIR nucleic acid is a modified form of DNA or RNA. Non-limiting examples of modifications include substitutions of a naturally-occurring base, modified backbones, sugar or internucleoside (backbone) linkage with a modified base, and modified sugars. In one embodiment, the purified NIR nucleic acid is a recombinant DNA incorporated into a vector. In one embodiment, the purified NIR nucleic acid is a recombinant DNA incorporated into an automatically replicating plasmid or virus. In certain embodiments, the purified NIR nucleic acid is a recombinant DNA incorporated into the genomic DNA of a prokaryotic or eukaryotic host cell.

In certain embodiments, the NIR nucleic acid comprises at least one additional nucleic acid segment. In one embodiment, the additional nucleic acid segment is a fusion protein sequence (e.g., His-tag, FLAG-Tag, GST, GFP, an antibody fragment, e.g., an antibody Fc region, signal peptides, sequences for selection purposes, etc.). In one embodiment, the additional nucleic acid segment is a transcription regulator sequence (e.g., promoters, enhancers, and the like). In one embodiment, the additional nucleic acid segment is a linker sequence present at the 5′ end, 3′ end or at a location within the polypeptide encoding sequence. In certain embodiments, the NIR nucleic acids comprise one or more additional nucleic acid segments described herein, or any combinations thereof.

In any of the embodiments described herein, the NIR nucleic acid may be present in a suitable vector, including but not limited to a viral vector, a bacterial plasmid, or an artificial chromosome suitable for cloning and/or expression in a eukaryotic cell or cell extract, prokaryotic cell or cell extract, and/or combinations thereof. In a specific embodiment, the vector is a plasmid. In a specific embodiment, the vector is a viral vector. In a specific embodiment, the vector is capable of autonomous replication in a host cell into which it is introduced (e.g., bacterial vectors having a bacterial origin of replication and episomal mammalian vectors). In one embodiment, the vector is integrated into the genome of a host cell as a transgene upon introduction into the host cell, and thereby is replicated along with the host genome. In one embodiment, the vector described herein is a transgene. In a specific embodiment, the vector is an expression vector.

The recombinant expression vectors of the invention comprise an NIR nucleic acid operably linked to one or more regulatory sequences. Non-limiting examples of regulatory sequences include promoters, enhancers and other expression control elements (e.g., polyadenylation signals). In one embodiment, the regulatory sequence is a tissue specific regulatory sequence. The selection of regulatory sequences is made on the basis of the host cells to be used for expression, the level of expression of protein desired, etc. In one embodiment, the vector comprises the nucleotide sequence of an NIR nucleic acid operably linked to a promoter. In one specific embodiment, the promoter is a constitutive promoter. In another specific embodiment, the promoter is an inducible promoter. In a further specific embodiment, the promoter directs constitutive expression of the NIR nucleic acid in many types of cells. In one embodiment, the promoter directs tissue specific expression of the NIR nucleic acid in a particular cell type.

In any of the embodiments, the NIR nucleic acid can be present as: one or more naked DNAs; one or more nucleic acids disposed in an appropriate expression vector and maintained episomally; one or more nucleic acids incorporated into the host cell's genome; a modified version of an endogenous gene encoding the polypeptide described herein; one or more nucleic acids in combination with one or more regulatory nucleic acid sequences; or combinations thereof.

A vector for the production of the polypeptide molecule can be produced by recombinant DNA technology using techniques well-known in the art based on the nucleotide sequence of an NIR nucleic acid. In one embodiment, the recombinant expression vector is transcribed and translated in vitro. An expression vector can also be transferred to cells (e.g., host cells) by conventional techniques and the resulting cells can then be cultured by conventional techniques to produce an NIR polypeptide. In specific embodiments, the expression vectors express the NIR polypeptides in prokaryotic or eukaryotic cells. In one embodiment, the expression vectors of the invention are introduced into host cells to produce fusion proteins or polypeptides from the NIR nucleic acid.

A variety of host-expression vector systems can be utilized to express the NIR polypeptides and derivatives. In one embodiment, the NIR polypeptides or derivatives are produced by the host-expression vector system and are subsequently isolated or purified. The host-expression systems that can be used include but are not limited to microorganisms such as bacteria (e.g., E. coli and B. subtilis) transformed with recombinant bacteriophage DNA, plasmid DNA or cosmid DNA expression vectors containing coding sequences for polypeptides; yeast (e.g., Saccharomyces Pichia) transformed with recombinant yeast expression vectors containing coding sequences for polypeptides; plant cell systems (e.g., Chlamydomonas reinhardtii and other green algae) infected with recombinant virus expression vectors (e.g., cauliflower mosaic virus, CaMV; tobacco mosaic virus, TMV) or transformed with recombinant plasmid expression vectors (e.g., Ti plasmid) containing coding sequences for polypeptides; insect cell systems infected with recombinant virus expression vectors (e.g., baculovirus) containing coding sequences for polypeptides; or mammalian cell systems (e.g., BHK, CHO, COS, CRL7O3O, HEK 293, HeLa, HsS78Bst, MDCK, NIH 3T3, NS0, PER.C6, and VERO cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, the expression of an NIR nucleic acid is regulated by a constitutive promoter, inducible promoter or tissue specific promoter.

In bacterial systems, a number of expression vectors can be advantageously selected depending upon the use intended for the NIR polypeptides and derivative being expressed. For example, when a large quantity of an NIR polypeptide or derivative is to be produced, for the generation of pharmaceutical compositions of such polypeptide or derivative, vectors which direct the expression of high levels of fusion polypeptide products that are readily purified can be desirable. Such vectors include, but are not limited to, the E. coli expression vector pUR278 (Ruther et al., 1983, EMBO 12:1791), in which the polypeptide coding sequence can be ligated individually into the vector in frame with the lacZ coding region so that a fusion polypeptide is produced; pIN vectors (Inouye and Inouye, 1985, Nucleic Acids Res. 13:3101-3109; Van Heeke and Schuster, 1989, J. Biol. Chem. 24:5503-5509); and the like. pGEX vectors can also be used to express foreign polypeptides as fusion polypeptides with glutathione 5-transferase (GST). In general, such fusion polypeptides are soluble and can easily be purified from lysed cells by adsorption and binding to matrix glutathione agarose beads followed by elution in the presence of free glutathione. The pGEX vectors are designed to include protease cleavage sites so that the cloned target gene product can be released from the GST moiety.

In mammalian host cells, a number of viral-based expression systems can be utilized. In cases where an adenovirus is used as an expression vector, the polypeptide coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the polypeptide molecule in infected hosts (e.g., see Logan and Shenk, 1984, Proc. Natl. Acad. Sci. USA 8 1:355-359). Specific initiation signals can also be required for efficient translation of inserted polypeptide coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bittner et al., 1987, Methods in Enzymol. 153:51-544).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used.

For long-term, high-yield production of recombinant proteins, stable expression is preferred. For example, host cells stably expressing the NIR polypeptide or derivative can be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the NIR polypeptide or derivative.

Once an NIR polypeptide or derivative thereof has been produced by recombinant expression, it can be purified by any method known in the art for purification of polypeptides, for example, by chromatography (e.g., ion exchange, affinity, and sizing column chromatography), centrifugation, differential solubility, or by any other standard technique for the purification of proteins and polypeptides. Further, the NIR polypeptide or derivative can be fused to non-Serrate protein sequences described herein or otherwise known in the art to facilitate purification.

The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Gait (ed.) (1984) Oligonucleotide Synthesis: A Practical Approach, IRL Press; Ausubel et al., Current Protocols in Molecular Biology, John Wiley & Sons (1987 and annual updates); Current Protocols in Immunology, John Wiley & Sons (1987 and annual updates); Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press; Sambrook et al. (2001) Molecular Cloning: A Laboratory Manual, Cold Spring Harbor Laboratory Press, Cold Spring Harbor, N.Y.

5.3 Chemical Synthesis of NIR Polypeptides

The NIR polypeptides and derivatives thereof described herein also can be produced by any method known in the art for the synthesis of polypeptides, for example, by chemical synthesis (e.g., using a peptide synthesizer according to standard methods).

5.4 Antibodies to Serrate NIR

Antibodies to Serrate ELR4-6, or that recognize an epitope containing at least a portion of ELR4-6, within the Notch inhibitory region (NIR) of a Serrate protein are also provided. ELRs 4-6, as described below, are necessary for cis-inhibition of Notch signaling. The Serrate protein can be any Serrate protein as described above. Such antibodies, termed herein NIR antibodies, can be used to reduce the Notch signaling inhibition mediated by the NIR and thus increase Notch pathway activity.

As used herein, the terms “specifically binds” and “specifically recognizes”, are analogous terms in the context of antibodies and refer to molecules that specifically bind to an antigen (e.g., epitope) as understood by one skilled in the art. In one embodiment, an NIR antibody that specifically binds to an NIR polypeptide can bind to other peptides or polypeptides, generally with lower affinity as determined by, e.g., immunoassays, Biacore™, KinExA 3000 instrument (Sapidyne Instruments, Boise, Id.), or other assays known in the art.

In one embodiment, the NIR antibody is an immunoglobulin. An NIR antibody can be any one of the five major classes of immunoglobulins: IgA, IgD, IgE, IgG, and IgM, or subclasses (isotypes) thereof (e.g., IgA1, IgA2, IgG1, IgG2, IgG3, and IgG4), based on the identity of its heavy-chain constant domains referred to as alpha (α), delta (δ), epsilon (ε), gamma (γ) and mu (μ), respectively.

In specific embodiments, the NIR antibodies can be any of the following: intact monoclonal antibodies, intact polyclonal antibodies, linear antibodies, single chain antibodies (scFv), multispecific antibodies such as bispecific antibodies generated from at least two intact antibodies, multispecific antibodies formed from antibody fragments, monovalent or monospecific antibodies, chimeric antibodies, human antibodies, humanized antibodies, fusion proteins comprising an antigen determination portion of an antibody, and any other modified immunoglobulin molecule comprising an antigen recognition site so long as the antibodies exhibit the desired biological activity. In specific embodiments, the NIR antibodies can be naked or conjugated to other molecules. In specific embodiments, the NIR antibodies are conjugated to toxins or radioisotopes.

The NIR antibodies can be from any animal origin, including but not limited to mammals (e.g., sheep, rabbit, goat, guinea pig, mouse, donkey, cat, pig, rat, monkey, camel, dog, cow, hamster, human, or horse). In one embodiment, an NIR antibody is a murine antibody. In one embodiment, an NIR antibody is a human antibody. In one embodiment, an NIR antibody is an engineered antibody, for example, an antibody recombinantly produced.

In one embodiment, an NIR antibody is a monoclonal antibody. In contrast to polyclonal antibodies that typically include different antibodies directed against different antigenic determinants, a monoclonal antibody is a homogenous antibody population involved in the highly specific recognition and binding of a single epitope or antigenic determinant. Monoclonal antibodies can be prepared using a wide variety of techniques known in the art, including but not limited to by hybridoma, phage selection, recombinant expression, transgenic animals, or a combination thereof. In a particular embodiment, an NIR antibody is produced by, or obtained from, a hybridoma cell.

In one embodiment, an NIR antibody is a chimeric antibody. In one embodiment, a chimeric NIR antibody comprises different portions of the antibody that are derived from different immunoglobulin molecules. In one embodiment, portions of a chimeric NIR antibody are derived from different immunoglobulin molecules from different animal species. In one embodiment, a chimeric NIR antibody contains a variable region of a human monoclonal antibody fused to a constant region of a mouse antibody. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415.

In one embodiment, an NIR antibody is a humanized antibody. A humanized NIR antibody can be any forms of non-human (e.g., hamster, mouse, rat, rabbit, etc.) antibodies that are specific to Serrate ELR4-6, or that recognize an epitope containing at least a portion of ELR4-6, and contain minimal non-human sequences. In one embodiment, a humanized NIR antibody is a human immunoglobulin in which residues from the complementary determining regions (CDRs) are replaced by residues from a CDR (or CDRs) of a non-human species (e.g., hamster, mouse, rat, rabbit, etc.). In one embodiment, a humanized NIR antibody is a human immunoglobulin in which the human CDR residues and optionally some framework region (FR) residues are substituted by residues from analogous sites in an antibody from a non-human species (e.g., hamster, mouse, rat, rabbit, etc.). In one embodiment, a humanized NIR antibody comprises one or more non-human (e.g., hamster, mouse, rat, rabbit, etc.) CDRs and one or more human framework regions, and optionally human heavy chain constant region and/or light chain constant region. In one embodiment, a humanized NIR antibody comprises one or more primate framework regions. In one embodiment, a humanized NIR antibody comprises one or more non-human primate framework regions. Production of humanized antibodies can be performed using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), and chain shuffling (U.S. Pat. No. 5,565,332).

Also provided herein are antibody fragments of the above-described antibodies that maintain the ability to specifically bind to the epitope, and thus comprise the idiotype, or immunospecific binding region, of the antibody. In specific embodiments, an antibody fragment can maintain at least 50%, at least 60%, at least 70%, at least 80%, at least 90%, or at least 95% of the ability to specifically bind to an epitope. Non-limiting examples of antibody fragments are Fab fragments, Fab′ fragments, F(ab′)₂ fragments, bispecific Fab, a single chain Fab, bispecific single chain antibody fragments, disulfide-linked Fv (dsFv), bispecific dsFv, camelized VH, diabodies and triabodies. A whole antibody can be enzymatically cleaved by pepsin to produce a F(ab′)₂ fragment, or can be enzymatically cleaved by papain to produce two Fab fragments.

In specific embodiments, an NIR antibody or fragment thereof is isolated or purified. The NIR antibody or fragment thereof can be separated from cellular components of the cells from which it is isolated or recombinantly produced. The NIR antibody or fragment thereof can also be produced by chemical synthesis, and separated from chemical precursors or other chemicals which are involved in the synthesis of the antibody or fragment. In specific embodiments, an isolated or purified NIR antibody or fragment thereof is substantially free of cellular material and/or chemical precursors or other chemicals. In specific embodiments, an isolated or purified NIR antibody or fragment thereof is substantially free of other antibodies or fragments with different antigenic specificities than the isolated or purified antibody or fragment thereof. In specific embodiments, an isolated or purified NIR antibody or fragment thereof has less than about 30%, 25%, 20%, 15%, 10%, 5%, 2%, 1%, 0.5%, or 0.1% (by weight) of contaminating proteins (i.e., proteins other than the NIR antibody or fragment). In specific embodiments, the NIR antibody or fragment thereof is recombinantly produced and is substantially free of culture medium. In specific embodiments, the NIR antibody or fragment thereof is recombinantly produced and the culture medium represents less than about 20%, 10%, 2%, 1%, 0.5%, or 0.1% of the volume of the NIR antibody preparation. In specific embodiments, the NIR antibody or fragment thereof is produced by chemical synthesis and is substantially free of chemical precursors or other chemicals. In specific embodiments, the NIR antibody or fragment thereof is produced by chemical synthesis and has less than about 20%, 10%, 2%, 1%, 0.5%, or 0.1% (by weight) of chemical precursors or chemicals other than the NIR antibody or fragment thereof.

In specific aspects of any of the above-described types of antibodies, provided herein are NIR antibodies (e.g., isolated antibodies, monoclonal antibodies, chimeric antibodies, and humanized antibodies) and fragments thereof, that specifically bind Serrate ELR4-6, or that specifically recognize an epitope containing at least a portion of ELR4-6 of a Serrate protein, e.g., human Serrate-1 or human Serrate-2. The NIR antibodies and fragments thereof can bind to any combinations of ELR 4, ELR 5 and ELR6, or any amino acid regions and combinations thereof within the ELR4-6 region, or to linear or discontinuous epitopes comprising at least a portion of ELR4-6 and another Serrate sequence. In one embodiment, an NIR antibody or fragment thereof specifically binds to the ELR 4 region of a Serrate protein. In one embodiment, an NIR antibody or fragment thereof specifically binds to the ELR 5 region of a Serrate protein. In one embodiment, an antibody or fragment thereof described herein specifically binds to the ELR 6 region of a Serrate protein.

In one embodiment, an NIR antibody or fragment thereof specifically binds to a polypeptide consisting essentially of the ELR4-6 region of a Serrate protein, e.g., human Serrate-1 or human Serrate-2.

In one embodiment, the NIR antibody or fragment thereof increases Notch activation. In specific embodiments, the NIR antibody or fragment increases Notch activation by at least about 20%, 40%, 60%, 80%, 100%, 200% or 300%. Notch activation levels can be assessed by methods known to one of skill in the art (e.g., measurement of transcriptional activation by quantitative PCR of Notch target genes such as Hes and Hey).

In certain embodiments, the NIR antibody or fragment thereof does not specifically bind to regions other than ELR4-6 of a Serrate protein, e.g., human Serrate-1 or human Serrate-2. In certain embodiments, the NIR antibody or fragment specifically binds to ELR4-6 of a Serrate protein (e.g., human Serrate-1 or human Serrate-2) with higher affinity than to other regions of the Serrate protein.

In specific embodiments, an NIR antibody or fragment thereof specifically binds to ELR4-6 of Serrate from an animal (e.g., frog, rat, mouse, monkey, goat, donkey, dog, cat, rabbit, pig, human, or bird). In particular embodiments, an NIR antibody or fragment specifically binds to ELR4-6 of a mammalian Serrate protein. In particular embodiments, an NIR antibody or fragment specifically binds to ELR4-6 of a primate Serrate protein. In particular embodiments, an NIR antibody or fragment specifically binds to ELR4-6 of a human Serrate protein (e.g., human Serrate-1 or human Serrate-2).

The NIR antibodies and fragments thereof can be produced by any method known in the art for the synthesis of antibodies, for example, by chemical synthesis or by recombinant expression techniques.

5.5 Methods of Use

Provided herein are methods of use of the NIR polypeptides and derivatives (e.g., as described hereinabove); NIR nucleic acids (e.g., as described hereinabove); vectors expressing the NIR polypeptides or derivatives (e.g., as described hereinabove); host cells comprising the vectors expressing the NIR polypeptides or derivatives (e.g., as described hereinabove); and the NIR antibodies (e.g., as described hereinabove). In certain aspects, methods described herein can be used to antagonize or inhibit Notch signaling. In certain aspects, methods described herein can be used to agonize or activate Notch signaling. In certain aspects, methods described herein can be for in vitro, in vivo, or ex vivo uses.

5.5.1 Regulation of Notch Signaling In Vitro or Ex Vivo

In certain aspects, provided herein are uses of the NIR antibody or fragment thereof to agonize or activate Notch signaling in a cell in vitro or ex vivo. In certain aspects, provided herein are used of the NIR polypeptide or derivative to antagonize or inhibit Notch signaling in a cell in vitro or ex vivo.

In one embodiment, the NIR antibodies or fragments thereof are used to activate Notch signaling and thereby inhibit differentiation in precursor cells. Such precursor cells can optionally be expanded. Notch regulates the competence of many different cell types to respond to more specific signals, with the particular cell fates chosen depending upon the developmental history of each cell type and the specific signaling pathways operating within it. When Notch function is activated in a precursor cell (e.g., progenitor or stem cell), the precursor cell can be prevented from differentiating even in the presence of the correct differentiation signals. Since Notch signaling activity may not destroy or, preferably, does not substantially impair, the ability of precursor cells to divide, precursor cells may be expanded, ex vivo in order to provide a source of precursors which are useful in gene therapy as well as tissue repair. Once, however, Notch function activation subsides, the cells can respond again to developmental cues. Methods of using Notch signaling to regulate cell differentiation in vitro or ex vivo are described in International Publication Number WO 97/11716, which is incorporated by reference herein in its entirety.

Precursor cells can be obtained by any method known in the art. Without being bound by any theory, the cells can be obtained directly from tissues of an individual or from cell lines or by production in vitro from less differentiated precursor cells, e.g., stem or progenitor cells. An example of obtaining precursor cells from less differentiated cells is described in Gilbert, 1991, Developmental Biology, 3rd Edition, Sinauer Associates, Inc., Sunderland Mass. Non-limiting examples of precursor cells are mesenchymal stem cells, neural stem cells, fetal cells, hematopoietic stem cells, liver stem cells, and kidney stem cells.

In one embodiment, the NIR antibody or fragment thereof is used to maintain a precursor cell in a particular differentiation state in order to provide indefinitely, or for a given period of time, a chemical produced by a cell of that differentiated state, to a particular tissue. In one embodiment, the NIR antibody or fragment thereof is administered to a precursor cell for a long period of time (e.g., hours or days). In one embodiment, the precursor cell is encapsulated with a solution of the NIR antibody or fragment thereof.

In one embodiment, the NIR antibody or fragment thereof is used to treat an ex vivo cell population, which is then allowed to proliferate in culture before being transplanted. In another embodiment, the NIR antibody or fragment thereof is used to treat a cell population ex vivo, which is then directly transplanted without necessarily being allowed to proliferate in vitro. In one embodiment, after treatment by the NIR antibody or fragment thereof, a Notch signaling antagonist is used to reverse or neutralize the action of the NIR antibody or fragment thereof. In one embodiment, the Notch signaling antagonist is an NIR polypeptide or derivative.

Regarding use of Notch agonists to inhibit differentiation in precursor cells, permitting expansion of the cells, see also U.S. Pat. No. 7,399,633, U.S. Pat. No. 6,337,387, Fre et al., 2005, Nature 435:964-968, Ohishi et al., 2002, J. Clin. Invest. 110:1165-1174, Pui et al., 1999, Immunity 11:299-308, Varnum-Finney et al., 1998, Blood 91:4084-4091, which are incorporated by reference herein in their entireties.

Notch signaling is involved in the pathogenesis of a variety of human tumors. As in differentiation, its effect is probably context-specific, inhibiting transformation in some tissues and promoting malignancy in others. Therefore, Notch can function as an oncoprotein in some human tumors and as a tumor suppressor in others. Some cancers are characterized by an increased expression of Notch and/or an increase in Notch pathway activity, compared to such expression or activity in normal, non-malignant cells. Such increases in the expression of Notch and/or Notch pathway activity in the tumors can be due to gain-of-function Notch mutations or ligand-mediated activation of the Notch pathway. Non-limiting examples of tumors with increased Notch pathway activity and/or expression of Notch include: T lymphoblastic leukemias/lymphomas (T-ALL) (Allenspach et al., 2002, Cancer Biol. Ther. 1:466-476; Louvi and Artavanis-Tsakonas, 2012, Semin. Cell Dev. Biol. [Epub ahead of print], PMID:22373641[PubMed]), a subset of diffuse large B-cell lymphomas, mature B-cell lymphomas and diffuse large B-cell lymphomas (Louvi and Artavanis-Tsakonas, 2012, Semin. Cell Dev. Biol. [Epub ahead of print], PMID:22373641 [PubMed]), breast cancer (e.g. mammary carcinomas) (Allenspach et al., 2002, Cancer Biol. Ther. 1:466-476; U.S. Pat. No. 6,083,904), lymphoproliferative disorders (e.g., chronic lymphocytic leukemias, Hodgkin's lymphomas and anaplastic large cell lymphomas) (Allenspach et al., 2002, Cancer Biol. Ther. 1:466-476), colon cancer (U.S. Pat. No. 6,083,904; PCT Publication No. WO 94/07474), some cervical cancers (U.S. Pat. No. 6,083,904; PCT Publication No. WO 94/07474), medulloblastomas (Koch and Radtke, 2007, Cell Mol. Life Sci. 64:2746-2762), renal cell carcinomas (Sjolund et al., 2008, J Clin Invest. 118:217-228) and pancreatic cancer (Koch and Radtke, 2007, Cell Mol. Life Sci. 64:2746-2762). In these tumors, strategies to antagonize or inhibit Notch pathway activity can be utilized in preclinical studies and/or cancer treatment.

In one embodiment, the NIR polypeptide or derivative is used to antagonize or inhibit Notch signaling in a cell in vitro for preclinical studies. In one embodiment, the cell is isolated from a patient tissue sample suspected of being pre-neoplastic. In one embodiment, the cell is isolated from a tissue sample from a patient diagnosed with cancer. In specific embodiments, the pre-neoplastic lesion or cancer is characterized by increased Notch pathway activity and/or expression of Notch. In specific embodiments, the cells isolated from a patient tissue sample are grown in vitro and exposed to an NIR polypeptide or derivative or nucleic acid, and responses of the cells to the NIR polypeptide or derivative or nucleic acid are evaluated. In one embodiment, where exposure to an NIR polypeptide or derivative or nucleic acid results in a cell phenotype that is more normal (i.e., less representative of a pre-neoplastic state, neoplastic state, malignant state, or transformed phenotype), that polypeptide or derivative or nucleic acid is indicated for therapeutic use.

There are also human cancers which are characterized by a decreased expression of Notch and/or a decrease (or downregulation) in Notch pathway activity, compared to such expression or activity in normal, non-malignant cells. Non-limiting examples of tumors with decreased Notch pathway activity and/or expression of Notch include: lung cancer (e.g., small-cell lung cancer), prostate cancer (e.g., prostate adenocarcinomas), skin cancer (e.g., basal cell cancer, cutaneous squamous cell carcinomas) and neuroblastomas (Allenspach et al., 2002, Cancer Biol. Ther. 1:466-476; Louvi and Artavanis-Tsakonas, 2012, Semin. Cell Dev. Biol. [Epub ahead of print], PMID:22373641 [PubMed]). In these tumors, strategies to agonize or activate Notch pathway activity can be utilized in preclinical studies and/or cancer treatment.

In one embodiment, the NIR antibody or fragment thereof is used to agonize or activate Notch signaling in a cell in vitro for preclinical studies. In one embodiment, the cell is isolated from a patient tissue sample suspected of being pre-neoplastic. In one embodiment, the cell is isolated from a tissue sample from a patient diagnosed with cancer. In specific embodiments, the pre-neoplastic lesion or cancer is characterized by decreased Notch pathway activity and/or expression of Notch. In specific embodiments, the cells isolated from a patient tissue sample are grown in vitro and exposed to an NIR antibody or fragment thereof, and responses of the cells to the NIR antibody or fragment thereof are evaluated. In one embodiment, where exposure to an NIR antibody or fragment thereof results in a cell phenotype that is more normal (i.e., less representative of a pre-neoplastic state, neoplastic state, malignant state, or transformed phenotype), that antibody or fragment thereof is indicated for therapeutic use.

Many assays standard in the art can be used to assess whether a pre-neoplastic state, neoplastic state, or a transformed or malignant phenotype, is present. A transformed phenotype can be a set of in vitro characteristics associated with a tumorigenic ability in vivo. Characteristics associated with a transformed phenotype can include a more rounded cell morphology, looser substratum attachment, loss of contact inhibition, loss of anchorage dependence, release of proteases such as plasminogen activator, increased sugar transport, decreased serum requirement, expression of fetal antigens, disappearance of the 250,000 dalton surface protein, etc. (see, Luria et al., 1978, General Virology, 3d Ed., John Wiley & Sons, New York pp. 436-446; U.S. Pat. No. 5,869,282; U.S. Pat. No. 6,083,904).

The preclinical results on cells of a patient tissue sample can indicate how the patient will respond to a certain therapeutic strategy, e.g., activation or inhibition of Notch signaling, and therefore can be used to guide selection and/or exclusion of patients for the therapeutic strategy.

In other specific embodiments, the in vitro assays described supra can be carried out using a cell line (see, U.S. Pat. No. 5,869,282; U.S. Pat. No. 6,083,904), rather than a cell sample derived from the specific patient to be treated, in which the cell line is derived from or displays characteristic(s) associated with the malignant, neoplastic or pre-neoplastic disorder desired to be treated or prevented, or is derived from other cell types upon which an effect is desired, according to the present invention.

In additional embodiments of the invention, in any or all of the ex vivo or in vitro methods described hereinabove, the following polypeptides and derivatives thereof (derivatives being as described in Section 5.1 above for the NIR polypeptides) can be used instead of or in addition to the NIR polypeptides or derivatives: polypeptides comprising the DSL domain, the DOS domain (ELRs 1-2), and the ELR4-6 domains of a Serrate protein; and in a specific embodiment, polypeptides comprising (i) the amino terminus through the DOS domain of a Serrate protein, and (ii) the ELR4-6 domains of said Serrate protein. In further embodiments, the foregoing polypeptides lack the transmembrane domain and the intracellular domain, and optionally an ELR other than ELRs 1-2 and ELRs 4-6.

5.5.2 Therapeutic and Prophylactic Uses

The invention provides for treatment or prevention of disorders in which Notch signaling is involved by administration of a therapeutic agent of the invention. Such therapeutic agents (termed herein “Therapeutics”) include: NIR polypeptides and derivatives (e.g., as described hereinabove); NIR nucleic acids (e.g., as described hereinabove); vectors comprising the NIR nucleic acids (e.g., as described hereinabove); host cells expressing the NIR nucleic acids (e.g., as described hereinabove); and NIR antibodies (e.g., as described hereinabove). Thus, the invention provides for treatment or prevention of diseases involving increased or decreased Notch expression or activity relative to normal cells in a subject in need thereof comprising administering a Therapeutic. Such normal cells can be obtained or derived from an individual (e.g., human) lacking the disease or from normal tissue of an individual having or suspected of having a disease in which Notch signaling is involved (as described below).

In one embodiment, Therapeutics which antagonize, or inhibit, Notch signaling (Notch pathway function) (termed herein “Antagonist Therapeutics”) are administered for therapeutic effect. In another embodiment, Therapeutics which agonize, or activate, Notch signaling (Notch pathway function) (termed herein “Agonist Therapeutics”) are administered for therapeutic effect.

In specific embodiments, the Antagonist Therapeutic is an NIR polypeptide or derivative, an NIR nucleic acid, a vector comprising an NIR nucleic acid, or a host cell expressing an NIR nucleic acid. In a specific embodiment, the Agonist Therapeutic is an NIR antibody or fragment thereof. Suitable in vitro or in vivo assays may be utilized to determine the effect of a specific Therapeutic and whether its administration is indicated for treatment of the affected tissue, since the developmental history of the tissue may determine whether an Antagonist or Agonist Therapeutic is desired.

As used herein, the terms “treat”, “treatment” and “treating” refer to the reduction or amelioration of the progression, severity, and/or duration of a disorder or disease (e.g., cancer) or symptom thereof.

The subject to be treated is preferably a mammal such as a non-primate (e.g., cows, pigs, horses, cats, dogs, goats, rabbits, rats, mice, etc.) or a primate (e.g., monkey and human), most preferably a human. In one embodiment, the subject is a mammal, preferably a human, which has a disorder or disease in which Notch signaling (Notch pathway function) is involved or which has been diagnosed with such a disorder or disease. In a specific embodiment, the subject is a non-human primate.

As used herein, the terms “a disorder in which Notch signaling is involved” or “a disease in which Notch signaling is involved” are used interchangeably and refer to any disease or its symptoms that is completely or partially caused by, is dependent on, or is the result of, or can be alleviated or inhibited by modulation of Notch pathway expression and/or activity.

In certain aspects, a disorder or disease in which Notch signaling is involved can be characterized by an increased expression of Notch, Delta or Serrate/Jagged, or an increase in Notch pathway activity, compared to such expression or activity in normal cells. In certain aspects, a disorder or disease in which Notch signaling is involved can be characterized by a decreased expression of Notch, Delta or Serrate/Jagged, or a decrease in Notch pathway activity, compared to such expression or activity in normal cells. In particular embodiments, Notch expression or activity which is higher or lower than the Notch expression or activity in normal cells leads to cellular transformation, neoplasia, and tumorogenesis. In certain aspects, a disorder or disease in which Notch signaling is involved is characterized by the presence of a mutation in Notch, Delta or Serrate/Jagged, or any of the other Notch pathway genetic components.

Protein expression levels in the affected cells from a subject with a disorder or disease in which Notch signaling is involved can be assessed by methods known to one of skill in the art. Many methods standard in the art can be thus employed, including but not limited to immunoassays to detect and/or visualize proteins (e.g., Western blot, immunoprecipitation followed by sodium dodecyl sulfate polyacrylamide gel electrophoresis, immunocytochemistry, etc.) and/or hybridization assays to detect protein expression by detecting and/or visualizing respectively mRNA (e.g., Northern assays, dot blots, in situ hybridization, etc.). In particular embodiments, Notch, Delta or Serrate/Jagged expression in an affected cell from a subject with a disorder or disease in which Notch signaling is involved is at least about 10%, 20%, 30%, 40%, 50%, 60%, 70%, 80%, 90%, or 100% higher or lower than that in a normal cell (e.g., a cell expressing normal levels of Notch, Delta or Serrate/Jagged). In particular embodiments, the average Notch, Delta or Serrate/Jagged expression in a population of affected cells from a subject with a disorder or disease in which Notch signaling is involved is at least about 10%, 20%, 30%, 40%, 50%, 60%, 70%, 80%, 90%, or 100% higher or lower than that in a normal cell population (e.g., a cell population expressing normal levels of Notch, Delta or Serrate/Jagged).

Notch activity levels can be assessed by methods known to one of skill in the art (e.g., measurement of transcriptional activation by quantitative PCR of Notch target genes such as Hes and Hey). In particular embodiments, Notch activity in an affected cell from a subject with a disorder or disease in which Notch signaling is involved is at least about 10%, 20%, 30%, 40%, 50%, 60%, 70%, 80%, 90%, or 100% higher or lower than that in a normal cell (e.g., a cell containing normal Notch activity). In particular embodiments, Notch activity in a population of affected cells from a subject with a disorder or disease in which Notch signaling is involved is at least about 10%, 20%, 30%, 40%, 50%, 60%, 70%, 80%, 90%, or 100% higher or lower than that in a normal cell population (e.g., a cell population containing normal Notch activity).

In one aspect, the invention relates to a method for inhibiting Notch activity in a subject, comprising administering to the subject the Antagonist Therapeutics of the invention. Non-limiting examples of Notch activity can include Notch pathway effects upon differentiation, Notch receptor signaling, and transcriptional activation of a Notch target gene (e.g., Hes and Hey). In a specific embodiment, administering an Antagonist Therapeutic to the subject is sufficient to inhibit Notch activity in the subject (e.g., in a cell or cell population of the subject) by at least about 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein and/or known to one of skill in the art (e.g., measurement of transcriptional activation by quantitative PCR of Notch target genes such as Hes and Hey).

In one aspect, the invention relates to a method for increasing Notch activity in a subject, comprising administering to the subject the Agonist Therapeutics of the invention. Non-limiting examples of Notch activity can include Notch pathway effects upon differentiation, Notch receptor signaling, and transcriptional activation of a Notch target gene (e.g., Hes and Hey). In a specific embodiment, administering an Agonist Therapeutic to the subject is sufficient to increase Notch activity in the subject (e.g., in a cell or cell population of the subject) by at least about 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein and/or known to one of skill in the art (e.g., measurement of transcriptional activation by quantitative PCR of Notch target genes such as Hes and Hey).

The Agonist and Antagonist Therapeutics of the invention have therapeutic utility for a disorder in which Notch signaling is involved. In one aspect, a disorder or disease in which Notch signaling is involved are known to one of skill in the art and include the diseases and disorders described below.

Some disorders or diseases are characterized by an increased expression of Notch, Delta or Serrate/Jagged, and/or an increase in Notch pathway activity, compared to such expression or activity in normal cells. Thus, in specific embodiments, such disorders or diseases are treated or prevented by administering an effective amount of an Antagonist Therapeutic, e.g., an NIR polypeptide or derivative, that antagonizes Notch pathway function. Non-limiting examples of disorders or diseases that can be treated or prevented by administering an effective amount of an Antagonist Therapeutic are: T lymphoblastic leukemias/lymphomas (T-ALL) (Allenspach et al., 2002, Cancer Biol. Ther. 1:466-476; Louvi and Artavanis-Tsakonas, 2012, Semin. Cell Dev. Biol. [Epub ahead of print], PMID:22373641 [PubMed]), a subset of diffuse large B-cell lymphomas, mature B-cell lymphomas and diffuse large B-cell lymphomas (Louvi and Artavanis-Tsakonas, 2012, Semin. Cell Dev. Biol. [Epub ahead of print], PMID:22373641 [PubMed]), breast cancer (e.g., mammary carcinomas) (Allenspach et al., 2002, Cancer Biol. Ther. 1:466-476; U.S. Pat. No. 6,083,904), lymphoproliferative disorders (e.g., chronic lymphocytic leukemias, Hodgkin's lymphomas and anaplastic large cell lymphomas) (Allenspach et al., 2002, Cancer Biol. Ther. 1:466-476), colon cancer (U.S. Pat. No. 6,083,904; PCT Publication No. WO 94/07474), some cervical cancers (U.S. Pat. No. 6,083,904; PCT Publication No. WO 94/07474), medulloblastomas (Koch and Radtke, 2007, Cell Mol. Life Sci. 64:2746-2762), renal cell carcinomas (Sjolund et al., 2008, J Clin Invest. 118:217-228), pancreatic cancer (Koch and Radtke, 2007, Cell Mol. Life Sci. 64:2746-2762); and metabolic bone diseases (e.g., Hajdu-Cheney syndrome and Serpentine fibula polycystic kidney syndrome) (Louvi and Artavanis-Tsakonas, 2012, Semin. Cell Dev. Biol. [Epub ahead of print], PMID:22373641 [PubMed]).

Some other disorders or diseases are characterized by a decreased expression of Notch, Delta or Serrate/Jagged, and/or a decrease in Notch pathway activity, compared to such expression or activity in normal cells. Thus, in specific embodiments, such disorders or diseases are treated or prevented by administering an effective amount of an Agonist Therapeutic, e.g., an NIR antibody or fragment thereof, that agonizes Notch pathway function. Non-limiting examples of disorders or diseases that can be treated or prevented by administering an effective amount of an Agonist Therapeutic are: lung cancer (e.g., small-cell lung cancer), prostate cancer (e.g., prostate adenocarcinomas), skin cancer (e.g., basal cell cancer, cutaneous squamous cell carcinomas), neuroblastomas (Allenspach et al., 2002, Cancer Biol. Ther. 1:466-476; Louvi and Artavanis-Tsakonas, 2012, Semin. Cell Dev. Biol. [Epub ahead of print], PMID:22373641 [PubMed]); hereditary pleiotropic diseases, e.g., Alagille syndrome (Louvi and Artavanis-Tsakonas, 2012, Semin. Cell Dev. Biol. [Epub ahead of print], PMID:22373641 [PubMed]); skeletal disorders, e.g., spondylocostal dysostosis (Chapman et al., 2011, Hum. Mol. Genet. 20:905-916); and cerebrovascular diseases, e.g., cerebral autosomal dominant arteriopathy with subcortical infarcts and leukoencephalopathy (CADASIL) (Louvi and Artavanis-Tsakonas, 2012, Semin. Cell Dev. Biol. [Epub ahead of print], PMID:22373641 [PubMed]).

Additional diseases or disorders, cells of which type can be tested in vitro (and/or in vivo) with an Antagonist or an Agonist Therapeutics described herein, and upon observing the appropriate assay result, can be treated or prevented by administering an effective amount of the Antagonist or the Agonist Therapeutic according to the present invention. These diseases or disorders include but are not limited to cancers, e.g., a subset of leukemias, acute leukemia, acute lymphocytic leukemia, acute myelocytic leukemia, myeloblastic leukemia, promyelocytic leukemia, myelomonocytic leukemia, monocytic leukemia, erythroleukemia, a subset of chronic leukemias, chronic myelocytic (granulocytic) leukemia, polycythemia vera, a subset of lymphomas, non-Hodgkin's disease, multiple myeloma, Waldenstrom's macroglobulinemia, heavy chain disease, a subset of solid tumors, a subset of sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, Leiomyosarcoma, rhabdomyosarcoma, ovarian cancer, a subset of squamous cell carcinomas, adenocarcinoma, sweat gland carcinoma, sebaceous gland carcinoma, papillary carcinoma, papillary adenocarcinomas, cystadenocarcinomna, medullary carcinoma, bronchogenic carcinoma, hepatoma, bile duct carcinoma, choriocarcinoma, seminoma, embryonal carcinoma, Wilms' tumor, testicular tumor, lung carcinoma, small cell lung carcinoma, bladder carcinoma, epithelial carcinoma, glioma, astrocytoma, craniopharyngioma, ependymoma, pinealoma, hemangioblastoma, acoustic neuroma, oligodendroglioma, menangioma, melanoma, and retinoblastoma (see, U.S. Pat. No. 5,869,282; for a review of such disorders, see Fishman et al., 1985, Medicine, 2d Ed., J. B. Lippincott Co., Philadelphia).

In certain embodiments, a cancer which is treated according to the present invention is metastatic, or an advanced cancer which has spread outside the organ or site of origin, either by local invasion or metastasis. In particular embodiments, the cancers which is treated according to the present invention is a recurrent cancer which has regrown, either at the initial site or at a distant site, after a response to initial therapy, e.g., after surgical removal of the tumor.

Non-limiting examples of non-cancer diseases or disorders in which Notch signaling is involved, and which can be treated according to the present invention include: hereditary pleiotropic diseases, skeletal disorders, metabolic bone diseases, cardiovascular diseases (e.g., tetralogy of Fallot and pulmonic stenosis), cerebrovascular diseases (e.g., schemic stroke and vascular dementia) and nervous system disorders (Louvi and Artavanis-Tsakonas, 2012, Semin. Cell Dev. Biol. [Epub ahead of print], PMID:22373641 [PubMed]; U.S. Pat. No. 5,869,282).

In certain embodiments, the nervous system disorder in which Notch signaling is involved, and which is treated according to the present invention is a nervous system injury, or a disease or disorder which result in either a disconnection of axons, a diminution or degeneration of neurons, or demyelination (see, U.S. Pat. No. 5,869,282). Nervous system lesions which may be treated in a patient (including human and non-human mammalian patients) according to the invention include but are not limited to the following lesions of either the central (including spinal cord, brain) or peripheral nervous systems: (1) traumatic lesions, including lesions caused by physical injury or associated with surgery, for example, lesions which sever a portion of the nervous system, or compression injuries; (2) ischemic lesions, in which a lack of oxygen in a portion of the nervous system results in neuronal injury or death, including cerebral infarction or ischemia, or spinal cord infarction or ischemia; (3) malignant lesions, in which a portion of the nervous system is destroyed or injured by malignant tissue which is either a nervous system associated malignancy or a malignancy derived from non-nervous system tissue; (4) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis; (5) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis; (6) lesions associated with nutritional diseases or disorders, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration; (7) neurological lesions associated with systemic diseases including but not limited to diabetes (diabetic neuropathy, Bell's palsy), systemic lupus erythematosus, carcinoma, or sarcoidosis; (8) lesions caused by toxic substances including alcohol, lead, or particular neurotoxins; and (9) demyelinated lesions in which a portion of the nervous system is destroyed or injured by a demyelinating disease including but not limited to multiple sclerosis, human immunodeficiency virus-associated myelopathy, transverse myelopathy or various etiologies, progressive multifocal leukoencephalopathy, and central pontine myelinolysis (see, U.S. Pat. No. 5,869,282).

For diseases or disorders that are not yet known whether an Agonist Therapeutic or an Antagonist Therapeutic should be used, in vitro assays can be performed to determine which Therapeutic has therapeutic utility. In one embodiment, a subject tissue sample is grown in culture, and exposed to a Therapeutic, and the effect of such Therapeutic upon the tissue sample is observed. In one embodiment, where the subject has a malignancy, a sample of cells from such malignancy is plated out or grown in culture, and the cells are then exposed to a Therapeutic. A Therapeutic which inhibits the survival or growth of the malignant cells (e.g., by promoting terminal differentiation) is selected for therapeutic use in vivo. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, etc. In a specific aspect, the malignant cell cultures are separately exposed to (1) an Agonist Therapeutic, and (2) an Antagonist Therapeutic; the result of the assay can indicate which type of Therapeutic has therapeutic efficacy (see, U.S. Pat. No. 5,869,282).

In one embodiment, the effective doses of the Therapeutics can be extrapolated from dose-response curves derived from in vitro or animal model test systems. In one embodiment, the effective doses of the Therapeutics to a subject are determined by the skilled physician. In some embodiments, a single dose is administered one or more times to a subject to prevent or treat a disorder or disease in which Notch is involved (e.g., cancer).

In some embodiments, the Therapeutics of the invention are administered, e.g., hourly, daily, weekly, biweekly (i.e., once every two weeks), monthly, bimonthly, or trimonthly. In some embodiments, the Therapeutics of the invention are administered to a subject in cycles, wherein the Therapeutics of the invention are administered for a period of time, followed by a period of time when the Therapeutics are not administered.

In some embodiments, the Therapeutics of the invention are administered in combination with one or more additional therapies to a subject in need thereof. In some embodiments, the therapies are administered serially, sequentially, concurrently, or concomitantly. In one embodiment, the therapies are administered in different compositions, separately. In some embodiments, the Therapeutics of the invention are administered in combination with one or more cancer therapies. The Therapeutics can be administered in combination with any therapeutic or prophylactic agent which is useful, has been used, or is currently being used for the prevention or treatment of cancer in a subject. Non-limiting examples of cancer therapies include chemotherapy, radiotherapy, surgery, gene therapy, immunotherapy, tyrosine kinase inhibitors, histone deacetylase inhibitor, and γ-secretase inhibitor. In a particular embodiment, the Therapeutic administered in combination with one or more cancer therapies achieves a synergistic effect.

In one aspect, the invention provides methods of gene therapy using the NIR nucleic acids and the vectors expressing the NIR nucleic acids. In specific embodiments, disorders or diseases characterized by an increased expression of Notch, Delta or Serrate/Jagged, and/or an increase in Notch pathway activity (such as those disorders or diseases described above) are treated or prevented by gene therapy comprising administering an effective amount of NIR nucleic acids or vectors expressing the NIR nucleic acids to a subject in need thereof. The NIR nucleic acids are expressed in the subject for therapeutic effect. Such disorders or diseases include but are not limited to various cancers as described above.

Introduction of the NIR nucleic acids or the vectors comprising the NIR nucleic acids into cells of a host can be achieved by various methods known in the art. Those methods include, but are not limited to, direct injection of naked DNA constructs, bombardment with gold particles loaded with constructs, and macromolecule-mediated gene transfer using, e.g., liposomes, biopolymers, and the like. Preferred methods use gene delivery vehicles derived from viruses, including but not limited to adenoviruses, retroviruses, lentiviruses, herpes simplex viruses, vaccinia viruses, pox viruses and adeno-associated viruses. Because of the much higher efficiency as compared to, e.g., vectors derived from retroviruses, vectors derived from adenoviruses (so-called adenoviral vectors) are the preferred gene delivery vehicles for transferring nucleic acid molecules into host cells in vivo. The gene therapy described herein can be a somatic gene therapy or a germ line gene therapy.

In a particular aspect, the present invention provides a recombinant adenoviral vector that carries an NIR nucleic acid that encodes an NIR polypeptide or derivative thereof. In one embodiment, the NIR nucleic acid is provided in a format that allows expression of the NIR polypeptide or derivative in the cells of a subject in need thereof. In one embodiment, an NIR nucleic acid is operably linked to a regulatory sequence (e.g., promoter and/or enhancer) upstream, and is operably linked to a eukaryotic polyadenylation signal downstream. In a specific embodiment, the regulatory sequence is derived from the adenovirus used to construct an adenoviral vector, or alternatively from a different adenovirus. In a specific embodiment, the regulatory sequence is of exogenous origin. Useful regulatory sequences can be derived from, e.g., the Cytomegalovirus Immediate Early promoter/enhancer or the Rous Sarcoma Virus LTR promoter/enhancer, but can also be derived from other promoters active in mammalian or human cells that are known in the art. In one preferred embodiment, the NIR nucleic acid encodes an NIR polypeptide or derivative thereof of a mammalian Serrate protein. In another preferred embodiment, the NIR nucleic encodes an NIR polypeptide or derivative thereof of a human Serrate protein. The recombinant adenoviral vectors can be derived from any wild-type adenovirus serotype that allows the expression of the NIR polypeptides or derivatives in a subject in need thereof. In one embodiment, the recombinant adenoviral vector is derived from human adenovirus type. Methods for the construction of recombinant adenoviral vectors according to the invention and for their propagation in useful packaging cells have been described in patent application publications EP 0 707 071 and WO97/00326, incorporated herein by reference in their entireties. Other examples of vectors and packaging systems useful in the invention include, but are not limited to, those given in patent application publications WO 93/19191, WO 94/28152, WO 96/10642, and WO 97/04119. In one embodiment, a recombinant adenoviral vector expressing an NIR polypeptide or derivative is used to treat cancer in a subject. In a specific embodiment, the recombinant adenoviral vector is administered to the circulation of a subject in need thereof, preferably a mammal, and further preferably a human.

The invention also provides prophylactic methods, wherein the Therapeutics are administered to prevent the diseases and disorders described above.

In additional embodiments of the invention, in any or all of the therapeutics and prophylactic methods described hereinabove, any of the following polypeptides and derivatives thereof (derivatives being as described in Section 5.1 above for the NIR polypeptides) can be used instead of or in addition to the NIR polypeptides or derivatives: polypeptides comprising the DSL domain, the DOS domain (ELRs 1-2), and the ELR4-6 domains of a Serrate protein; and in a specific embodiment, polypeptides comprising (i) the amino terminus through the DOS domain of a Serrate protein, and (ii) the ELR4-6 domains of said Serrate protein. In further embodiments, the foregoing polypeptides lack the transmembrane domain and the intracellular domain, and optionally an ELR other than ELRs 1-2 and ELRs 4-6.

5.6 Pharmaceutical Compositions and Kits

Provided herein are pharmaceutical compositions and kits comprising the Therapeutics described herein, or any combinations thereof. In specific embodiments, provided herein is a pharmaceutical composition comprising a prophylactically or therapeutically effective amount of the Therapeutics described herein, and further comprising a pharmaceutically acceptable carrier. In particular aspects, compositions described herein can be for in vitro, in vivo, or ex vivo uses.

As used herein, the term “pharmaceutically acceptable” means being approved by a regulatory agency of the Federal or a state government, or listed in the U.S. Pharmacopeia, European Pharmacopeia or other generally recognized Pharmacopeia for use in animals, and more particularly in humans.

The carrier in the pharmaceutical composition can be a diluent, adjuvant (e.g., MPL, immunostimulatory oligonucleotides, Freund's complete and incomplete, mineral gels such as aluminum hydroxide, surface active substances such as lysolecithin, pluronic polyols, polyanions, peptides, oil emulsions, keyhole limpet hemocyanins, dinitrophenol, and potentially useful adjuvants for humans such as BCG (Bacille Calmette-Guerin) and Corynebacterium parvum), excipient, or vehicle with which the Therapeutic is administered. Such pharmaceutical carriers can be sterile liquids, such as water and oils, including those of petroleum, animal, vegetable or synthetic origin, such as soybean oil, peanut oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents: These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of the Therapeutics, preferably in purified form, together with a suitable amount of carrier so as to provide the form for proper administration to the patient. The formulation should suit the mode of administration.

In one embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic to ease pain at the site of the injection.

Generally, the ingredients of compositions of the invention are supplied as a kit either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In another embodiment, a kit of the invention further comprises a needle or syringe, preferably packaged in sterile form, for injecting the composition, and/or a packaged alcohol pad. Instructions are optionally included for administration of the compositions of the invention by a clinician or by the patient.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the composition of the invention which will be effective in the treatment or prevention of one or more symptoms associated with a disease or a disorder in which Notch signaling is involved can be determined by standard clinical techniques. The precise dose to be employed in the formulation will depend on the route of administration, the age of the subject, and the seriousness of the disease or disorder, and should be decided according to the judgment of the practitioner and each patient's circumstances. Effective doses may be extrapolated from dose-response curves derived from in vitro or animal model test systems.

For antibodies or fragments thereof, the therapeutically or prophylactically effective dosage administered to a subject is typically 0.1 mg/kg to 200 mg/kg of the subject's body weight. Preferably, the dosage administered to a subject is between 0.1 mg/kg and 20 mg/kg of the subject's body weight and more preferably the dosage administered to a subject is between 1 mg/kg to 10 mg/kg of the subject's body weight.

Treatment of a subject with a therapeutically or prophylactically effective amount of a Therapeutic that is an NIR polypeptide or derivative can include a single treatment or, preferably, can include a series of treatments. The compositions described herein can be administered at once, or can be divided into a number of smaller doses to be administered at intervals of time. In a preferred example, a subject is treated with an NIR polypeptide or derivative Therapeutic in the range of between about 0.1 to 30 mg/kg body weight, one time per week for between about 1 to 10 weeks, preferably between 2 to 8 weeks, more preferably between about 3 to 7 weeks, and even more preferably for about 4, 5, or 6 weeks. In a preferred example, a subject is treated with an NIR polypeptide or derivative Therapeutic in the range of between about 0.1 to 30 mg/kg body weight, one time per week for between about 1 to 10 weeks, preferably between 2 to 8 weeks, more preferably between about 3 to 7 weeks, and even more preferably for about 4, 5, or 6 weeks. In other embodiments, the pharmaceutical composition of the invention is administered once a day, twice a day, or three times a day. In other embodiments, the pharmaceutical composition is administered once a week, twice a week, once every two weeks, once a month, once every six weeks, once every two months, twice a year or once per year. It will also be appreciated that the effective dosage of the Therapeutics used for treatment may increase or decrease over the course of a particular treatment.

More than one of the Therapeutics can be administered simultaneously. In one embodiment, the Therapeutics are administered at different times. Compositions, such as those described herein, can also contain more than one active agents (for example, molecules, e.g., polypeptides or antibodies described herein) as necessary for the particular disease or disorder being treated. In certain embodiments, compositions comprise the Therapeutics described herein and one or more active agents with complementary activities that do not adversely affect each other. Such agents are suitably present in combination in amounts that are effective for the purpose intended. Such combination therapy can be administered to the patient serially or simultaneously or in sequence.

In certain aspects, the Therapeutic provided herein is included in the pharmaceutically acceptable carrier in an effective amount sufficient to exert a therapeutically useful effect in the absence of, or with minimal or negligible, undesirable side effects on the patient treated. A therapeutically effective concentration can be determined empirically by testing in in vitro and in vivo systems using routine methods and then extrapolated therefrom for dosages for humans.

The compositions provided herein can also be formulated to be targeted to a particular tissue, receptor, or other area of the body of the subject to be treated. Many such targeting methods are well known to those of skill in the art. All such targeting methods are contemplated herein for use in the instant compositions.

Also provided herein is a pharmaceutical pack or kit comprising one or more containers filled with one or more of the ingredients of the pharmaceutical compositions described herein, such as one or more of the polypeptide or derivative, the nucleic acid, the vector, the antibody or the fragment thereof provided herein. Optionally associated with such container(s) can be a notice in the form prescribed by a governmental agency regulating the manufacture, use or sale of pharmaceuticals or biological products, which notice reflects approval by the agency of manufacture, use or sale for human administration.

### 6. EXAMPLE

This example shows that ELRs 4-6 define a Notch inhibitory region in Serrate.

A systematic analysis of the extracellular domain of Ser to localize sequences involved in Notch inhibition was performed. EGF-like repeats 4 through 6 (ELRs 4-6) were identified as sequences that are not necessary for Notch trans-activation but are indispensable for Notch cis-inhibition. Consistent with these observations, the region defined by ELRs 4, 5 and 6 is conserved among Ser (Jagged) family ligands in other species. The same EGF-like repeat region is responsible for both cis-inhibition and Notch inhibitory properties associated with secreted forms of the Ser ligand. Moreover, forms lacking the ability to inhibit Notch still require endocytosis in order to activate Notch, thereby indicating that these deleted forms do not mimic an activated ligand form.

6.1 Results

6.1.1 Targeted Deletion Analysis

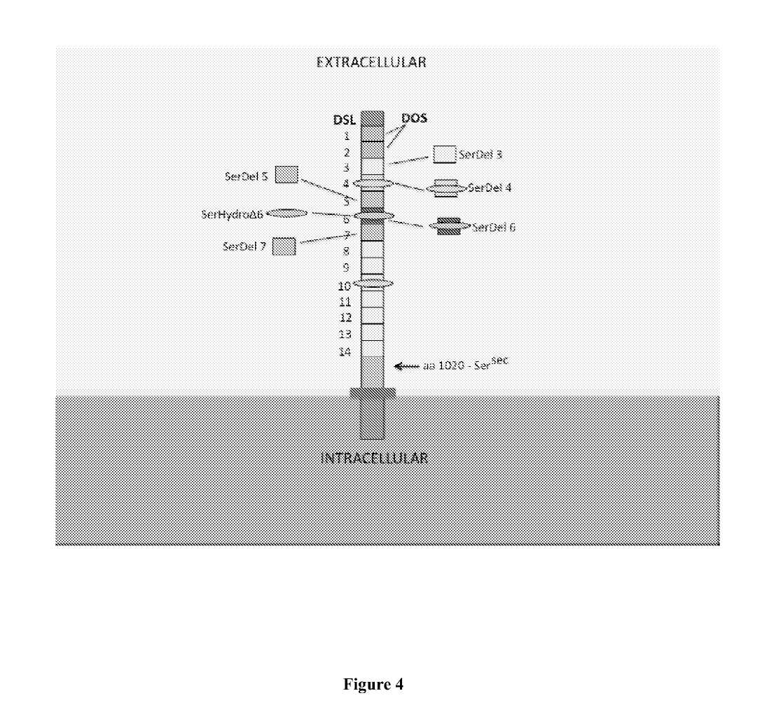

To define potential inhibitory domains in the extracellular domain of the Notch ligands, a systematic deletion analysis of an extracellular region of the Drosophila Notch ligand Ser was performed. Ser has 14 ELRs in its extracellular domain (FIG. 4) and has been the ligand best explored for its inhibitory properties (Klein et al., 1997, Dev. Biol. 1:123-134; Li and Baker, 2004, BMC Dev. Biol. 5). Transgenic fly lines carrying individual deletions for ELRs were generated and the constructs were expressed either via the patched Gal4 driver (Gal4ptc) (Hinz et al., 1994, Cell 1:77-87) at the anterior/posterior (AP) border of the wing or by a partial Ser gene promoter Gal4^{Ser2} (Hukriede et al., 1997, Development 17:3427-3437), which drives expression along the marginal region in the dorsal domain of the wing disc. Using the Gal4ptc promoter, the influence of ectopic Ser expression on Notch activity was measured by examining how it affects the expression of CUT, a transcriptional target of Notch signals and is a classic indicator of Notch activity in the dorsal/ventral boundary and ventral region of the wing disc (Doherty et al., 1996, Genes Dev. 4:421-434).

Ser constructs were expressed in the third instar imaginal wing disc via the ptc promoter using the Gal4 system (Brand and Perrimon, 1993, Development 2:401-415). Notch activity was assessed by examination of CUT expression induced in response to Notch activity (Neumann and Cohen, 1996, Development 11:3477-3485). Cells expressing Ser did not express CUT, in spite of having endogenous Notch expression. This is an indication that in these cells Ser expression inhibited Notch receptor activation (cis-inhibition; (de Celis and Bray, 1997, Development 17:3241-3251; Micchelli et al., 1997, Development 8:1485-1495); and see below). In contrast, Ser expression triggered Notch activity (trans-activation) on the anterior and posterior sides of the ptc stripe in the ventral region of the disc, i.e., in cells expressing the receptor and apposed to cells expressing the wild type ligand. Expression of the transgene carrying a deletion of ELR 7 (SerDel7), behaved indistinguishably from wild type. Therefore, ELR 7 is neither required for transactivation nor for cis-inhibition of Notch in this assay. A transgene lacking ELR 6 (SerDel6) driven by Gal4ptc trans-activated Notch comparably to expression of wild type Ser. However, cis-inhibition of Notch was nearly eliminated (CUT is expressed within the Ser expressing stripe). Thus, ELR 6 is necessary for cis-inhibition of Notch but not for trans-activation.

Within ELR 6 of Drosophila Ser there exists a stretch of non-EGF-like hydrophobic amino acids that is not conserved in Ser-like molecules outside of insects (Fleming et al., 1990, Genes Dev. 12A:2188-2201; Thomas et al., 1991, Development 3:749-761). To test if this hydrophobic region is required for cis-inhibition by Ser, the sequence of only this hydrophobic region was removed to generate the SerhydroΔ6 transgene (see, Materials and Methods in Section 6.3). When SerhydroΔ6 was expressed by Gal4ptc, trans-activation was observed adjacent to the ptc stripe but CUT activity was not observed within the ptc expression stripe. Thus, SerhydroΔ6 still retained both Notch trans-activation and cis-inhibition roles. To further define the cis-inhibiting region of Ser, ELR 5 or ELR 4 was deleted to generate the SerDel5 or SerDel4 transgene, and these transgenes were expressed via Gal4ptc. In both cases, transactivation of Notch occurred as with wild type Ser expression, but no cis-inhibition was observed as in the case of the ELR 6 deletion.

Transactivation of Notch was not observed when a deletion removing only ELR 3 (SerDel3tom) was generated and expressed driven with Gal4ptc. An inserted fluorescent tomato tag (Shaner et al., 2004, Nat. Biotechnol. 12:1567-1572) in the intracellular domain of SerDel3tom placed at an identical position to a tomato tag inserted into the wild type Ser construct demonstrates that the construct is expressed. Expression of SerDel3tom by Gal4ptc however did show reduced, variable Notch cis-inhibition. These results show that ELR3 is necessary for Notch trans-activation, and is involved, at least to some degree, in cis-inhibition.

It has been observed that weakly expressed forms of wild type Ser can lead to irregular activation of CUT within the ptc expression domain (Fleming et al., 1997, Development 15:2973-2981). Therefore, the expression of the deleted Ser forms was examined using the Gal4^{Ser2} promoter (Hukriede and Fleming, 1997, Genetics 2:359-374). Expression of either wild type Ser or SerDel7 via the Gal4^{Ser2} promoter (FIG. 5A) produced a notched margin phenotype and wing vein deltas in adult wings (FIGS. 5C, 5D). These phenotypes are characteristic of the intrinsic Notch-inhibiting dominant-negative effect that has been associated with excessive expression of wild type Ser, as seen in the Ser^{D} mutant allele or triggered by wild type Ser transgene mis-expression (Klein et al., 1997, Dev. Biol. 1:123-134; Thomas et al., 1995, Genetics 1:203-213). Similarly, expression by Gal4^{Ser2} of SerhydroΔ6, which retained the cis-inhibition property (see above), caused wing margin loss similar to that seen by wild type Ser (FIG. 5F). These findings support the conclusion that the hydrophobic amino acids within repeat six are not required for the cis-inhibition effect. In contrast, when SerDel6, SerDel5 or SelDel4 was expressed via Gal4^{Ser2}, no notching of the wing margins or wing vein deltas was observed; however, severe wing vein loss was observed within the expression domain of this promoter (FIG. 5E). Therefore, Gal4^{Ser2} expression of these constructs generated a novel phenotype distinct from the expression of wild type Ser forms. Given that only cis inhibition is affected by the ELR 6 deletion, this wing vein loss phenotype is likely due to abnormal activation of Notch within the vein forming territories leading to the specification of intervein cell fates (Huppert et al., 1997, Development 17:3283-3291). Finally, expression of SerDel3tom by Gal4^{Ser2}, demonstrates wing margin loss and delta formation of wing veins comparable to wild type expression in this pattern (FIG. 5I) supporting the conclusion that SerDel3tom retains the ability to cis inhibit Notch.

The above analyses limit the cis-inhibitory region of Ser to ELRs 4 through 6, and therefore repeats 4-6 of Ser were termed the Notch Inhibitory Region (NIR). To address whether or not the NIR is sufficient for the cis-inhibitory function, a construct (NIRtom; see Materials and Methods in Section 6.3) that expresses only ELRs 4, 5 and 6 followed by a tomato (Shaner et al., 2004, Nat. Biotechnol. 12:1567-1572) was expressed to follow expression efficiency. This construct produces a soluble protein that is secreted from cells (data not shown). Expression of NIRtom by the Gal4^{Ser2} promoter failed to generate any detectable phenotype in the adult wing (FIGS. 5J and 5K). These results demonstrate that the NIR is necessary but not sufficient for Notch inhibition.

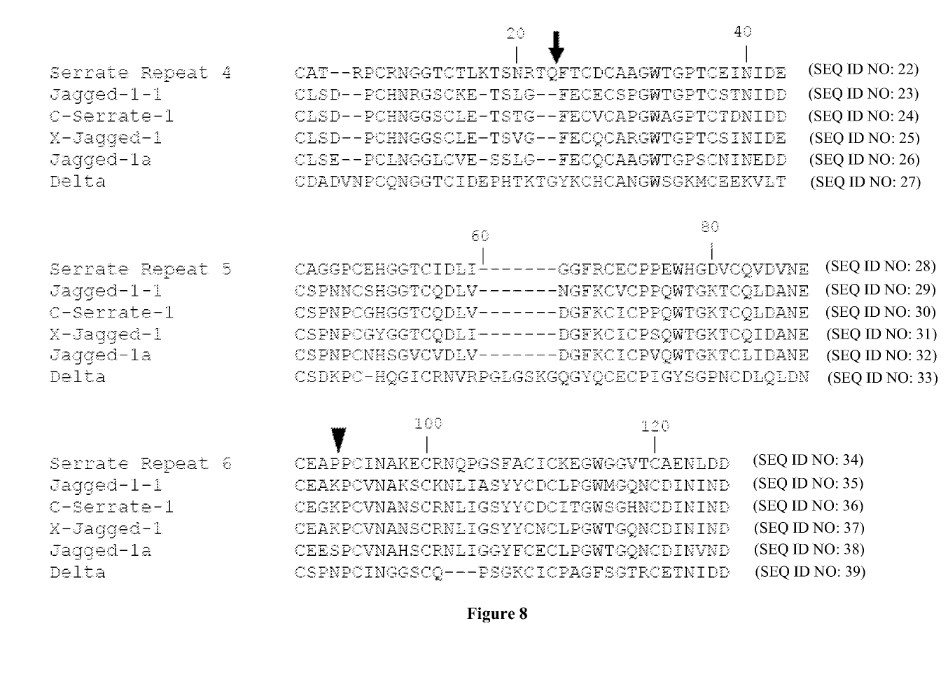

Given that Notch inhibition by its ligands is likely a general property and has been documented for both Ser and Delta ligands across species (Dorsky et al., 1997, Nature 6611:67-70; Ladi et al., 2005, J. Cell Biol. 6:983-992; Lowell and Watt, 2001, Mech. Dev. 1-2:133-140), comparison of the primary sequences around the NIR in the ligands was performed. Homology searches (FIG. 8), show that the sequences involved in cis-inhibition are well conserved within the Ser family of ligands but not between Ser and Delta.

6.1.2 Notch Activation by NIR Deficient Ser Depends on Endocytosis

Endocytosis seems to play a critical role within the signal-sending cell, as it may be essential for the re-cycling of ligands on the surface after they are presumably modified to generate a ligand form competent of interacting and activating the receptor (Wang and Struhl, 2004, Development 21:5367-5380). A critical element of the endocytic processes that allows ligand induced Notch trans-activation relies on the activity of epsin that is encoded by the liquid facets (lqf) gene in Drosophila (Overstreet et al., 2004, Development 21:5355-5366; Wang and Struhl, 2004, Development 21:5367-5380). Given that NIR-deleted Ser forms trans-activate but do not cis-inhibit Notch, we probed the potential role NIR plays in the lqf-dependent events that govern the generation of active, receptor competent ligands.

When wild type Ser is expressed in a normal lqf⁺ background, Notch is activated in the cells surrounding the clone but not within the clone, thereby demonstrating both trans-activation and cis-inhibition by the wild type form. In contrast, when wild type Ser is expressed in an lqf-deficient background, Notch activation is not observed around the clone. Epsin is thus, as expected, required in the ligand-expressing cell in order to activate Notch in the apposing cell (Overstreet et al., 2004, Development 21:5355-5366; Wang and Struhl, 2004, Development 21:5367-5380).

When analogous clones of the NIR-deficient SerDel6 form are expressed in an lqf⁺ background, Notch is activated both within and around the clone, consistent with retention of trans-activation and the loss of cis-inhibition for Notch by this form. Cells expressing SerDel6 in the lqf-deficient background can no longer activate Notch either within the same cell or in cells surrounding the clone. Thus, both the ability of the mutant ligand SerDel6, lacking NIR, to trans-activate as well as cis-inhibit still depends on the presence of lqf(epsin).

6.1.3 Dominant-Negative Ser Forms

Membrane bound or secreted mutant forms of Notch ligands that lack an intracellular (IC) domain have been shown to inhibit Notch signaling activity, although the mechanism(s) of such negative activity are not understood (Hukriede and Fleming, 1997, Genetics 2:359-374; Mishra-Gorur et al., 2002, J. Cell Biol. 2:313-324; Parks et al., 2000, Development 7:1373-1385; Qi et al., 1999, Science 5398:91-94; Sun and Artavanis-Tsakonas 1997, Development 17:3439-3448; Sun and Artavanis-Tsakonas 1996, Development 8:2465-2474). Given that the NIR region is retained in all known dominant-negative forms of Ser we sought to determine if this region is also responsible for the dominant-negative effects of Ser on Notch. We generated a Ser form termed Nterm6tom that expresses the N-terminal sequences of Ser through the end of ELR 6 followed by an in-frame fluorescent tomato tag to allow us to track expression of the construct (Shaner et al., 2004, Nat. Biotechnol. 12:1567-1572); see Materials and Methods in Section 6.3). As a control, we used a secreted, untagged, dominant-negative molecule called Ser^{sec} that is slightly longer than the known dominant-negative Bd^{G} form (encodes the N-terminus through amino acid 1020 of Ser and contains all 14 ELRs plus an additional 272 amino acids) (Hukriede and Fleming, 1997, Genetics 2:359-374; Hukriede et al., 1997, Development 17:3427-3437). The results of the expression of these molecules by the Gal4^{Ser2} promoter are shown in FIGS. 5L-O.

Expression of Ser^{sec} via the Gal4^{Ser2} driver (FIG. 5L) elicits a rudimentary wing, characteristic of dominant-negative ligand forms expressed in this pattern. Indistinguishable wing loss phenotypes are also associated with a similarly expressed NTerm6tom (FIG. 5M) transgene. This indicates that the dominant negative properties of this Ser form reside within the first 6 ELRs, an area encompassing the NIR. Given that ELRs 4 and 6 are necessary for cis inhibition, we refined the analysis by deleting either the 6^{th} (Ser^{sec}Del6) or the 4^{th} (Nterm6Del4) ELR of these secreted Ser molecules. Expression of these forms by Gal4^{Ser2} allowed us to determine that the loss of ELR 6 in Ser^{sec}Del6 greatly reduces the dominant-negative attributes of this molecule (FIG. 5N). Similarly, expression of Nterm6Del4 (FIG. 5O) demonstrates significantly reduced dominant-negativity of this secreted molecule. These findings demonstrate that the cis-inhibition domain NIR is critical for the dominant negative qualities of secreted Ser forms.

6.1.4 Cis- and Trans-Interactions Between Notch and Ser

Triggering the activation of the Notch receptor on the cell surface may depend on the competition between cis- and trans-interactions between Notch and its ligand. We chose to examine such competition by examining interactions between Notch and Ser expressing S2 tissue culture cells. It has been demonstrated that cells expressing Notch will physically bind and aggregate with cells expressing Ser or Delta (Fehon et al., 1990, Cell 3:523-534; Rebay et al., 1991, Cell 4:687-699). These interactions can be seen between adjacent cells (in trans) as judged by cell aggregation and by the co-localization of both molecules when expressed on the same cell (in cis). We reasoned that if the mechanism of Notch cis-inhibition results from competition between ligand and receptor in cis versus in trans, then interactions between Notch-expressing cells and Ser-expressing cells should be reduced or eliminated by co-expressing Ser in the cells that simultaneously express Notch.

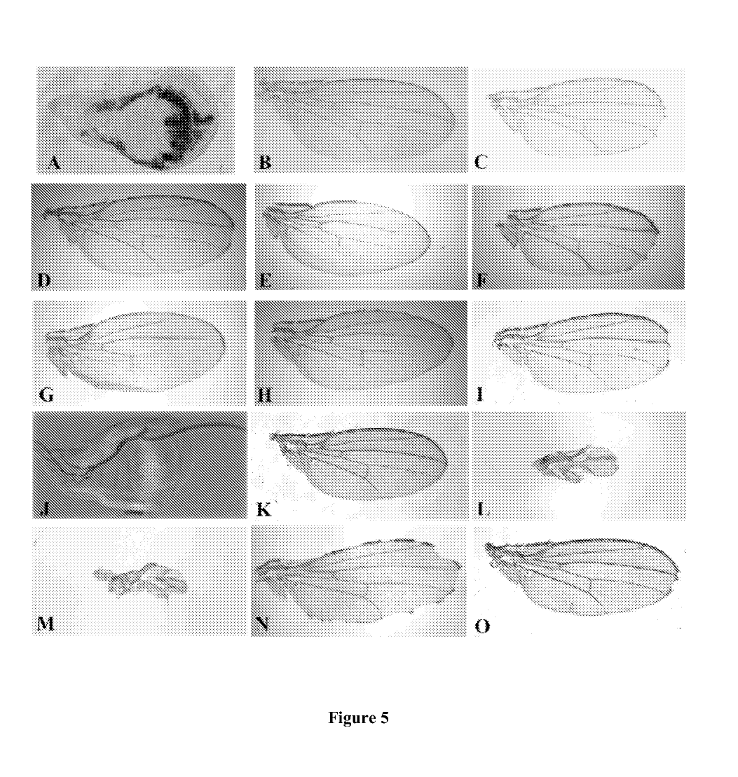

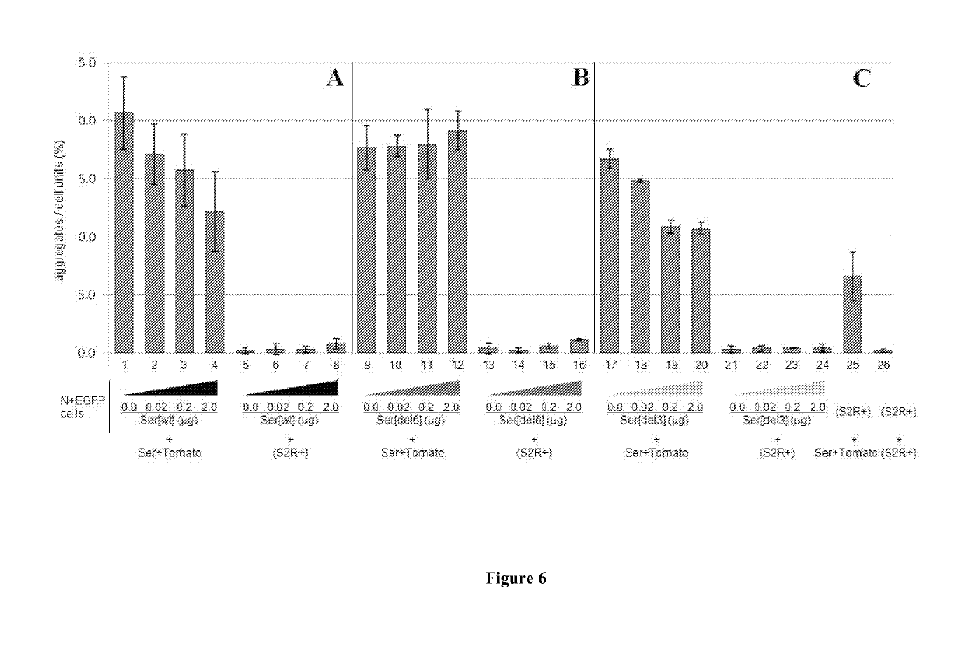

S2 cells stably expressing tomato-tagged Ser (Sertom) were mixed with S2 cells stably expressing EGFP labeled Notch and allowed to aggregate with one another to determine baseline levels of Notch-Ser interactions (FIG. 6). Subsequent experiments transiently transfected the stable Notch EGFP line with either Sertom (wild type), SerDel6tom or SerDel3tom transgenes at varying concentrations to promote cis-inhibition within the Notch-expressing cells. As seen in FIG. 6A, increasing levels of co-transfected wild type Sertom DNA in the Notch-expressing cells, effectively reduces the generation of cellular aggregates when these cells are mixed with Ser-expressing cells. This suggests that the cis-expression of Ser on Notch cells generates competition for Notch binding in trans, effectively reducing aggregation.

When SerDel6, a mutant that has little or no ability to cis-inhibit Notch in our wing expression assays (FIGS. 5A-O), is expressed in cis with Notch in S2 cells, aggregation is not inhibited (FIG. 6B). Thus this altered Ser form lacks the ability to compete in cis for Notch binding. To further examine this phenomenon, we repeated the experiment but used the SerDel3tom construct that fails to trans-activate Notch but demonstrates the ability to cis-inhibit Notch in vivo. When SerDel3tom was transfected into the Notch-expressing cells, we saw significantly reduced cellular aggregation (FIG. 6C) indicating that it can effectively compete in cis for Notch binding and demonstrates that the ability to trans-activate Notch can be separated from the ability to bind with Notch in trans. These observations also confirm that SerDel3 retains the functional NIR region of Ser.

6.1.5 Trans-Activation Versus Auto-Activation of Notch by SerDel6

Because both wild type Ser and the SerDel6 form appear to be capable of binding with and activating Notch, yet the SerDel6 form fails to cis-inhibit Notch, we sought to further examine the interaction of Notch and Ser in cis. It is reasonable to presume that the balance between cis and trans Notch-Ser interactions, between cells expressing both the ligand and the receptor, determines the directionality of the signal. The question of whether or not a ligand can activate the receptor on the same cell remains open and thus we used our constructs to probe this question. Although our studies clearly show that SerDel6 fails to cis-inhibit Notch, thereby allowing activation of Notch within the ligand-expressing stripe, there are always multiple, adjacent cells expressing ligand in these assays.

To determine if auto-activation of Notch is possible in this system, we generated single-cell clones in the wing disc capable of expressing wild type Ser or SerDel6 in an otherwise wild type background. As expected, a single cell expressing wild type Ser can only trans-activate Notch on adjacent cells, while keeping Notch inactive on its own cell surface. Thus, wild type Ser is incapable of auto-activating Notch when cis-inhibition is active in the ligand. When the non-inhibiting SerDel6 form is expressed in single-celled clones, we find that the results are comparable to wild type Ser expression. This demonstrates that Notch is not activated on the surface of a single cell expressing SerDel6. We conclude that a Ser molecule, whether it has an intact NIR or not, does not activate the Notch receptor when expressed in the same cell.

6.2 Discussion

In spite of the complexity of the genetic circuitry controlling Notch receptor activity, the developmental logic of the pathway is constant and linking, as a rule, the fate of one cell to that of the adjacent cellular neighbor. As two cells communicate utilizing Notch signals it is often, indeed usually the case that each cell initially expresses both the receptor and a ligand simultaneously. Therefore the pivotal decision that defines the receiving vs the signaling partner becomes a most important parameter. Elegant genetic data indicated that it is the ratio between receptor/ligand expressed in a cell that is guiding this decision and the possible functional significance of cis interactions was also first postulated on the basis of genetic evidence (Heitzler and Simpson, 1993, Development 3:1113-1123; Heitzler and Simpson, 1991, Cell 6:1083-1092). Early molecular indications (Fehon et al., 1990, Cell 3:523-534) pointed to trans as well as cis interactions between Notch and its ligands on the cell surface but the significance of the cis interactions or their relation to the trans mode of interactions remained unclear. Only recently, through the development of imaging approaches have we started gaining insight into how the receptor and the ligand interacts on the cell surface (Sprinzak et al., 2010, Nature 7294:86-90), reviewed in (D'Souza et al., 2010, Curr. Top. Dev. Biol. 73-129).

The importance of cis vs trans interactions and how these relationships serve the developmental logic of the Notch signaling pathway has been the central theme of the current study. This allowed us to define a molecular region in the extracellular domain of Ser, the ligand for which the most data exist implicating it in cis-interactions (Klein et al., 1997, Dev. Biol. 1:123-134; Li and Baker, 2004, BMC Dev. Biol. 5), which are essential for the cis-inhibitory signaling interaction with the Notch receptor. We show that cis interactions clearly inhibit the ability of the Notch receptor on the same cell to signal and that this property is dependent on the presence of the NIR that encompasses the extracellular area of Ser that includes ELRs 4, 5 and 6. Importantly, we find that the property of the ligand to inhibit in cis can be separated from its ability to activate in trans. Our results support the model where the interplay between cis and trans interactions between the ligand and the receptor on the same cell determine if this cell will be capable of receiving or transmitting the Notch signal.

6.2.1 Definition of the Notch Inhibitory Region

Our results indicate a noteworthy molecular modularity involving the ELRs in the extracellular domain of Ser. Our findings demonstrate that it is the ensemble of ELRs 4, 5 and 6 that is required for cis-inhibition of Notch yet removal of any of the individual repeats ELR 4 through EGF-like repeat 7 has no discernible effect on Notch trans-activation in our assays. This outcome does not strictly hold true for the SerhydroΔ6 construct. This construct, which removes only the hydrophobic, non-EGF-like sequences within EGF-like repeat 6, maintains the ability to cis-inhibit Notch but appears to have reduced Notch trans-activation capabilities as judged by the absence of anterior border staining when expressed via Gal4ptc. This reduction in Notch activation is not observed when the entirety of repeat 6 is deleted. We believe therefore that the reduction in Notch activation by SerhydroΔ6 is likely not to be physiologically significant.

While the cis-inhibitory properties of Ser seem to be determined by the NIR we must conclude that sequences mapping to the N-terminus of the NIR may also contribute to some degree. The first two ELRs of Ser constitute the DOS domain, a region shown to be necessary for Notch activation in other systems (Komatsu et al., 2008, PLoS Biol. 8: e196). Additionally, the N-terminal portion of human Jagged-1, which includes the DOS domain plus the third EGF-like repeat of that ligand, has been implicated in both cis- and trans-interactions with Notch (Cordle et al., 2008, Nat. Struct. Mol. Biol. 8:849-857). ELR3 is clearly required for Notch trans-activation in vivo and behaves accordingly in the cell aggregation assays. The lack of inhibition in the NIRtom construct demonstrates a requirement for at least some of these domains in addition to the NIR for Notch inhibition.

The above described results led to the finding that the NIR-deficient forms of Ser, like the wild type counterparts that are capable of trans-activation, still require endocytosis to activate Notch on the adjacent cell. Thus if the endocytosis-dependent step of the ligand that is thought to be necessary to produce a ligand form that can activate Notch modifies the ligand in some critical region (Wang and Struhl, 2004, Development 21:5367-5380), this modification must reside outside the NIR. Moreover, signal directionality from Ser does not depend on the NIR. Individual cells expressing either wild type or NIR-deleted Ser forms remain incapable of activating Notch on their own cell surfaces. We therefore presume that cis-inhibition does not reflect a need for the signal-sending cells to block auto-activation. It is rather likely that the requirement for endocytosis of ligand in signal-sending cells accounts for the directionality of the Notch signal.

The analysis of the Ser secreted forms implicates the NIR region in the dominant-negative effects of these molecules. We cannot however distinguish whether the inhibitory properties of the secreted dominant-negative ligand forms that lack the IC and transmembrane domains are limited to cis-interactions with Notch. Since they are secreted, it may be that these forms can interact with Notch on neighboring cells (in trans) as well as on their own cells (in cis) to produce Notch inhibition. While we do not know the cellular localization of the effects of secreted forms, we clearly associate the dominant-negative properties of these mutant forms to the same ELR region as cis-inhibition.

It has been demonstrated previously that Ser-induced Notch activation is limited to the ventral wing compartment due to the expression of fringe (fng) in the dorsal wing compartment (Panin et al., 1997, Nature 6636:908-912). Notch has been shown to be modified by fng such that Ser is unable to activate the fng-modified form of Notch dorsally but is able to activate unmodified Notch ventrally (Xu et al., 2007, J. Biol. Chem. 48:35153-35162). Conversely, Delta activates modified Notch dorsally but has greatly reduced ability to activate unmodified Notch ventrally. The specific regions of Ser and Delta that distinguish these interactions have not been fully defined but are at least in part encoded within the DSL domain of these ligands (Fleming et al., 1997, Development 15:2973-2981). Our findings revealed that individual removal of repeats 4 through 7 has no effect on the limitation of Ser activity to the ventral side. This suggests that the ligand function regulating interaction with fng-modified Notch either resides outside of repeats 4-7 or is redundant within these repeats since in no case of these deleted constructs do we observe Notch activation in the dorsal wing compartment.

6.2.2 Cis-Inhibition and Cell Aggregation

We extended the in vivo analyses by utilizing the classic Notch-ligand aggregation assays (Fleming et al., 1997, Development 15:2973-2981; Rebay et al., 1991, Cell 4:687-699) to examine whether or not ligand induced cis-inhibition interferes with cellular aggregation. The fact that wild type Ser DNA when transfected in a Notch-expressing cell inhibits the ability of this cell to aggregate with ligand expressing cells demonstrates that cis-expression of Ser on the Notch cell effectively competes with Notch-Ser interactions in trans. In contrast, the SerDel6 form that does not demonstrate cis-inhibition properties in vivo fails to inhibit aggregation of Ser and Delta expressing cells clearly indicating that the loss of cis-inhibition results from a failure of interaction between Ser and Notch on the same cell. Interestingly, when SerDel3, which fails to activate Notch in trans but retains its cis-inhibition properties, is used in this assay it can inhibit aggregation and hence it appears to retain the ability to compete for Notch interaction in cis.

The aggregation assays therefore support the notion that the activation and inhibition roles of Ser are separable properties of the ligand. They also raise the possibility that there exist two levels of Notch-ligand interaction. Aggregation likely depends on the most N-terminal regions of the ligands including the DSL and perhaps DOS domains (Cordle et al., 2008, Nat. Struct. Mol. Biol. 8:849-857; Komatsu et al., 2008, PLoS Biol. 8:e196). The possibility that both cis- and trans-interactions may be mediated by the N-terminal regions of the ligand in either of two orientations (Cordle et al., 2008, Nat. Struct. Mol. Biol. 8:849-857) could explain the cis/trans competition for binding that we observe in these assays. The first level of Notch-ligand interaction may simply mediate the association of ligand and receptor in either the cis or trans orientation. This interaction would be independent of either Notch activation (as demonstrated by SerDel3 competition) or Notch inhibition (as demonstrated by SerDel6 competition).

The second level of Notch-ligand interaction would entail the association of these ligands and receptors following binding. When associated in the trans-conformation, the alignment of ligand and Notch leads to dynamic Notch activation (requiring at least ELR3 of Ser). In contrast, when associated in the cis-conformation, the association of these molecules appears to generate a purposeful Notch inhibition that requires the presence of the NIR. This dual type interaction could explain the residual inhibition associated with secreted forms of Ser that lack NIR sequences (Ser^{sec}Del6 or Nterm6Del4). Clearly these secreted forms cannot activate Notch yet they must retain the ability to bind with Notch in order to produce their inhibitory effects. Both the Ser^{sec}Del6 and Nterm6Del4 constructs retain some degree of negative interaction with Notch even though ELR6 or ELR4 respectively, parts of the NIR region, have been removed. It is likely that the apparent remaining inhibitory activity associated with these molecules results from simple competition for Notch binding between these non-activating (and non-inhibiting) forms and endogenously expressed Notch ligands.

6.2.3 NIR Conservation in Ser but not in Delta Homologs

Cross species comparisons showed that ELRs 4 through 6 of Ser are conserved, consistent with the fact that the vertebrate ligands have been shown to cis-inhibit Notch in a manner similar to that seen in Drosophila (Chitnis et al., 1995, Nature 6534:761-766; Franklin et al., 1999, Curr. Biol. 24:1448-1457; Itoh et al., 2003, Dev. Cell. 1:67-82). Thus, we postulate that the NIR function is also likely to be conserved. These sequence comparisons failed to identify a comparable region in the Delta ligands even though Delta molecules display the common properties of Notch trans-activation and cis-inhibition (Jacobsen et al., 1998, Development 22:4531-4540; Klein et al., 1997, Dev. Biol. 1:123-134). Whether this reflects that a different region of Delta defines another functional equivalent of the Ser NIR or that the folded Delta protein has a structure that mimics the Ser NIR and adopts it function, remains to be determined.

The phenotypes we elicit when NIR-deleted forms are expressed in the wing discs using the Gal4^{Ser2} promoter, which is derived from the Ser promoter and expresses in most if not all of the cells that express Ser normally (Hukriede et al., 1997, Development 17:3427-3437) raise an intriguing possibility. The expression of these mutant forms cause loss of wing vein formation near the margins reminiscent of Notch Abruptex (Ax) mutations (Foster, 1975, Genetics 1:99-120; Portin, 1975, Genetics 1:121-133). The Ax mutations are often homozygous viable, dominant mutations that define ligand-dependent hyperactive gain of function Notch mutations (Heitzler and Simpson, 1993, Development 3:1113-1123; Portin, 1975, Genetics 1:121-133). The conspicuous and characteristic Ax phenotype is wing vein gapping similar to that seen when NIR deleted forms are expressed in the wing under the normal Ser expression pattern (Gal4^{Ser2}).

The similarity in phenotypes between the expression of these mutant forms of Ser and Notch Ax alleles, along with previous studies showing strong interactions between Ax alleles and Delta or Ser (de Celis and Garcia-Bellido, 1994, Genetics 1:183-194; de Celis and Bray, 2000, Development 6:1291-1302), raises the possibility that the NIR of Ser and the Ax regions of Notch are causally related. Ax mutations map to ELRs 24 through 29 of Notch (Hartley et al., 1987, EMBO J. 11:3407-3417; Kelley et al., 1987, Cell 4:539-548) and genetically fall into two classes: an enhancer class whose presence causes wing margin loss associated with Notch null alleles to become more pronounced when heterozygous to a Notch null allele and a suppressor class whose phenotype causes the wing margin loss to become less pronounced when heterozygous to a Notch null allele (Foster, 1975, Genetics 1: 99-120; Portin, 1975, Genetics 1:121-133). It is tempting to speculate that Ax mutations could represent Notch forms that are less responsive to ligand-induced cis-inhibition. If so, the Ax enhancer and suppressor classes of mutations could represent regions of Notch that interact with the Ser or the Delta NIR regions to cause inhibition. The mapped Ax enhancer mutations reside in ELR's 27-29 of Notch and the mapped Ax suppressor mutations map to ELRs 24 and 25 (Hartley et al., 1987, EMBO J. 11:3407-3417; Kelley et al., 1987, Cell 4:539-548). Furthermore if these different regions interact with separate ligands to induce Notch inhibition, then the ligand interaction domains of Ser and Delta need not show significant amino acid identity.

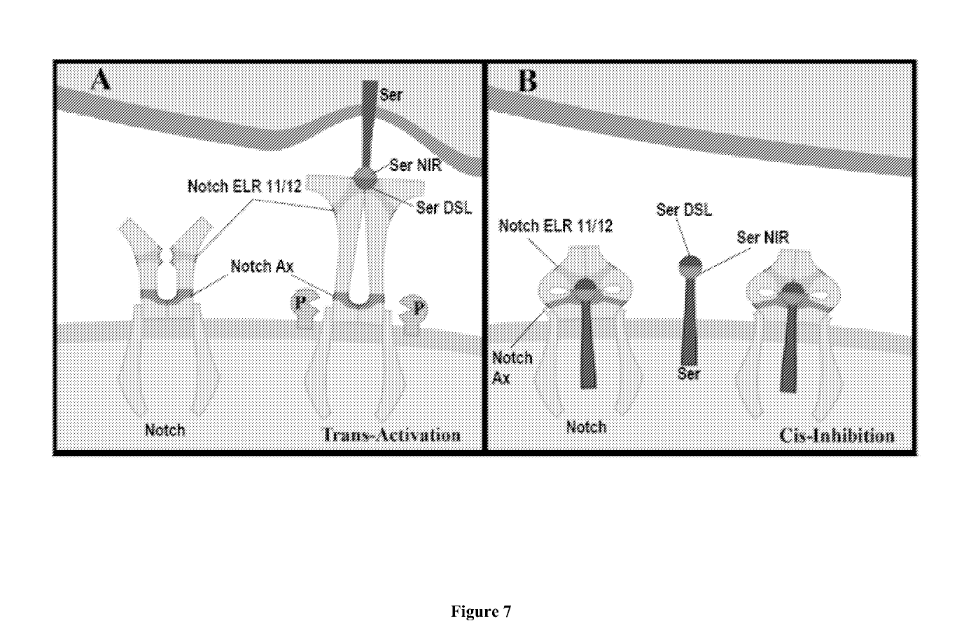

Our findings allow us to propose a simple model, which explains our observations and is consistent with the notion that the balance between cis and trans ligand interactions may ultimately provide directionality to the Notch signal. We interpret our findings to indicate differential interactions between Ser and Notch in cis and in trans as illustrated in FIG. 7. When Ser is expressed in trans to Notch, the interaction does not require the NIR and results in activation. In contrast, given that the NIR requires the DSL and N-terminal regions to mediate cis-inhibition, we propose that the N-terminal DSL region of Ser interacts with Notch, depicted as a dimer given the existing data, in cis and that this interaction is stabilized by further interaction of the NIR with Notch, possibly within the Ax domain. This stabilized cis-interaction is hypothesized to maintain Notch in an inactive state, recalcitrant to the crucial signal producing proteolytic cleavages.

6.3 Materials and Methods

6.3.1 Drosophila Cultures and Strains:

UAS-Ser expression constructs were generated and transgenic lines were produced by Genetic Services, Inc. Other stocks were obtained from the Bloomington Stock Center. Cultures were maintained on standard cornmeal/dextrose/agar medium supplemented with active dry yeast at 25° C. except when expressed by the Gal4ptc promoter (Hinz et al., 1994, Cell 1:77-87) or when generating single-celled clones where they were maintained at 18° C. Two copies of a Ser wing promoter (Gal4^{Ser2}; Hukriede and Fleming, 1997, Genetics 2:359-374) were use to express constructs along the dorsal/ventral wing boundary.

6.3.2 Construction of EGF-Like Repeat Deletions:

All constructs were generated from the wild type Ser cDNA sequence (Fleming et al., 1990, Genes Dev. 12A:2188-2201) and all sequence and amino acid numbers in the following constructs originate from that reference. Each construct was placed into the pUAST transformation vector (Brand and Perrimon, 1993, Development 2:401-415).

SerDel3:

Amino acids 351 through 390 deleted by cutting the Ser cDNA with Dra I (position 1484) and using the following primer to place an artificial Dra I site at position 1598 5′ CGAGATCGTTTAAATTTCTGTGCCACCAGGCCATGCCG CAACGGC 3′ (SEQ ID NO: 9) to create an in frame Dra I site at the beginning of EGF-like repeat 4.

SerDel4:

Amino acids 391 through 490 deleted by placing an artificial Ppu MI site at position 1619 of the Ser cDNA using the following primer: 5′ GCCGCCGTTGCGGCAGGGA CCCGTGGCACATGGGTGCTC 3′ (SEQ ID NO: 10) and fusing that with the Ppu MI site at position 1921.

SerDel5:

Amino acids 491 through 528 deleted by placing an artificial Kas I site at position 1916 of the Ser cDNA using the following primer: 5′ GCCACCATGCTCGCAGGGCG GCGCCTCGCACTCGTCGATATTTAT 3′ (SEQ ID NO: 11) and fusing that with the Kas I site at position 2031.

SerDel6:

Amino acids 529 through 610 deleted by placing an artificial Kas I site at position 2277 of the Ser cDNA using the following primer 5′ GACTGTGTGGCGC CGTGCCGGAATGGAGCC 3′ (SEQ ID NO: 12) and fusing that with the Kas I site at position 2031.

SerhydroΔ6:

Amino acids 532 through 574 deleted and Ile₅₇₅ changed to Leu₅₇₅ by using primer 5′ CCGGATCGATGCGGGAGCTCGCACTCGTTCACATCCA 3′ (SEQ ID NO: 13) to introduce a unique Sac I site at position 2029 and using primer 5′ CACATCCTTGGAGCTCGGACCCTGCATCAATGC 3′ (SEQ ID NO: 14) to introduce a unique Sac I site at position 2161 and then fusing the two halves of the cDNA at that Sac I site.

SerDel7:

Amino acids 611 through 647 deleted by placing an artificial Xba I site at position 2373 using primer 5′ GCGAGACGGATCTAGACGAGTGCGCCACTTCCC 3′ (SEQ ID NO: 15) and fusing that with the Xba I site at position 2262.

Ser^{sec} and Ser^{sec}Del6:

The region of Ser from the N-terminus through the first Bam HI site (position 3496) of the wild type cDNA was placed into the pUAST transformation to produce a secreted, extracellular form of Ser encoding all 14 ELRs plus an addition 72 amino acids. Ser^{sec}Del6 was produced by swapping the 5′ coding region of SerDel6 with the wild type 5′ end of Ser^{sec} at the Xba I site at position 2262.

Nterm6 and NTerm6Del4:

The N-terminal encoding region of Ser was taken from the wild type Ser cDNA and cut with Eco RI and Xba I at position 2262 near the end of EGF-like repeat 6 and fused in-frame with a tomato tag generated with the N-terminal encoding primer 5′ TGCCGAGAATCTAGATGACA TGGTGAGCAAGGGCGAGGAGGTC 3′ (SEQ ID NO: 16) containing a compatible 5′ Xba I site and a C-terminal encoding primer 5′ TGATCGGAATTCTTA CTTGTACAGCTCGTCCATGCC 3′ (SEQ ID NO: 17) with an in-frame stop codon followed by an Eco RI site. These two fragments were ligated into the pUAST vector for subsequent germ line transformation. The Deletion 4 Nterm6 construct was similarly constructed using the N-terminal encoding Eco RI to Xba I portion of the EGF-like Deletion 4 cDNA construct described above.

NIRtom:

A Bgl II site was introduced into the N-terminal region of Ser after the signal peptide using the primer 5′ GGTATTTGAGATTTCTAAGATCTCCAGCTCGAA GTTACC 3′ (SEQ ID NO: 18). The N-terminal segment was fused with the beginning of ELR 4 at amino acid 391 by generating a second Bgl II site with primer 5′ TGCGAGATCGTGGAGCAGATCTGTGCCACCAGGC CATGC 3′ (SEQ ID NO: 19). The construct was terminated following ELR 6 with the same tomato tag and fusion point as in the Nterm6 construct.

Tomato Tagged Ser:

A tomato tag (Shaner et al., 2004, Nat. Biotechnol. 12:1567-1572) was generated using the N-terminal primer 5′ GCTCCGGAAATGGTGAGCAAGGGCGAGGAG 3′ (SEQ ID NO: 20) and the C-terminal primer 5′ TAATTCCGGAGCCTTGTACAGCTCGTCCATGCC 3′ (SEQ ID NO: 21). The PCR fragment generated was cut with Bsp EI and inserted into the Bsp EI site of the Ser cDNA at position 4357.

6.3.3 Generation of Wing Disc Clones:

Epsin-Deficient Cell Clones:

Females of the genotype y w HsFLP1.22 TubP.Gal4 UAS.CD8-GFP/y w HsFLP1.22 TubP.Gal4 UAS.CD8-GFP; Tub.Gal80 FRT 2A/TM6B (provided by Dr. Gary Struhl) were crossed with either UAS-Ser*/UAS-Ser*; FRT 2A/FRT 2A males (where the * indicates either UAS-Ser (wild type) or UAS-SerDel6) to generate Ser expressing clones in a wild type background or to UAS-Ser*/UAS-Ser*; FRT 2A lqf^{SO11027}/TM6B males to generate similar clones in an epsin-background (Wang and Struhl 2005, Development 12, 2883-2894). Progeny of these crosses were heat shocked 24-36 hours after egg laying at 37° C. for 1 hour and then returned to 25° C. until crawling third instar stage for dissection and immunohistochemistry.

Single-Celled Clones:

By crossing w¹¹¹⁸; UASmCD8GFP; UASmCD8GFP; Act5C>y+>Gal4/UASmCD8GFP; Act5C>y+>Gal4 females (Lee and Luo, 1999, Neuron 3:451-461) to HsFLP1.22/Y; UAS-Ser*/UAS-Ser* males (where the * indicates either UAS-Ser (wild type) or UAS-SerDel6), animals capable of expressing a UAS-Ser form were produced. To induce single-celled clones, animals were raised at 18° C. and were heat-shocked at 37° C. for 1 hour during the early third instar stage. Wing discs were dissected from these animals 12-20 hours after heat shock and processed.

6.3.4 Immunohistochemistry:

Wing discs were dissected in PBS, fixed in 4% paraformaldehyde and blocked in 3% normal goat serum, 0.2% saponin in PBS. Primary antibodies were mouse anti-CUT (Developmental Studies Hybridoma Bank, DSHB) at 1:250. Secondary antibody was Alexa-Fluor 546 goat anti mouse IgG1 (Invitrogen) at 1:1000. GFP, EGFP and tomato expression were observed by intrinsic immunofluorescence.

6.3.5 S2 Aggregation Assays:

Cells stably expressing Notch+EGFP or Ser+Tomato were cultured in standard M3 medium (Gibco) supplemented with 10% fetal bovine serum and 100 mg/ml Hygromycin (Invitrogen) (Fehon et al., 1990, Cell 3:523-534). A different concentration (2.0, 0.2, 0.02, or 0.0 mg) of pMK33-Ser Bsp tom or pMK33-Del6 Ser Bsp tom was transfected into N+EGFP cells using Effectene (Qiagen). Equal amounts of plasmid were transfected by adding empty vector. One day after transfection, plasmid expression was induced with 0.35 mM CuSO₄. Four hours after induction began, the cells were mixed with Ser+Tomato cells in equal numbers, and allowed to aggregate overnight by rotating them on a cell rocker. Aggregates were defined as clusters of four or more cells. For all values, at least 100 cell units (single cells or cell clusters) were scored.

6.3.6 Sequence Alignments:

DNA sequences for Ser and Delta homologs were obtained through NCBI and were manually aligned using the CLC sequence Viewer (CLC Bio). The sequences used were Homo sapiens Jagged 1 (Jagged-1-1), U73936.1; Xenopus laevis jagged 1, NM_{—}001090307; Gallus gallus, C-Serrate-1, X95283; Danio rerio, jagged-1a, NM_{—}131861; Drosophila melanogaster delta, Y00222.

### INCORPORATION BY REFERENCE

Various references such as patents, patent applications, and publications are cited herein, the disclosures of which are hereby incorporated by reference herein in their entireties.

1. A polypeptide comprising the DSL domain, the DOS domain, and the ELR4-6 domains of a Serrate protein, wherein said polypeptide lacks at least one of the following domains of said Serrate protein: ELR3, ELR7, ELR8, ELR9, ELR10, ELR11, or ELR12.

2. A polypeptide comprising the amino terminus through the DOS domain of a Serrate protein, and the ELR4-6 domains of said Serrate protein, wherein said polypeptide lacks at least one of the following domains of said Serrate protein: ELR3, ELR7, ELR8, ELR9, ELR10, ELR11, or ELR12.

3. The polypeptide of claim 1 or 2, which lacks the ELR3 domain of said Serrate protein.

4. The polypeptide of claim 1 or 2, which lacks at least one of the following domains of said Serrate protein: ELR7, ELR8, ELR9, ELR10, ELR11, or ELR12.

5. The polypeptide of claim 3, wherein said polypeptide lacks each of the ELR3, ELR7, ELR8, ELR9, ELR10, ELR11, and ELR12 domains of said Serrate protein.

6. The polypeptide of claim 4, wherein said polypeptide lacks each of the ELR7, ELR8, ELR9, ELR10, ELR11, and ELR12 domains of said Serrate protein.

7. The polypeptide of claim 1 or 2, which lacks all ELRs except ELR4-6 of said Serrate protein.

8. The polypeptide of claim 1 or 2, which lacks all ELRs except ELR3-6 of said Serrate protein.

9. The polypeptide of claim 1 or 2, which further comprises ELR3 of said Serrate protein.

10. The polypeptide of claim 1 or 2, which comprises a fragment of said Serrate protein that spans the amino terminus through ELR6, ELR7, ELR8, ELR9, ELR10 or ELR11.

11. The polypeptide of claim 1, wherein said polypeptide lacks the transmembrane domain and the intracellular domain of said Serrate protein.

12. The polypeptide of claim 1, wherein the amino acid sequence of said polypeptide consists of the DSL domain, the DOS domain, and the ELR4-6 domains of a Serrate protein.

13. The polypeptide of claim 1, wherein the amino acid sequence of said polypeptide consists of the DSL domain, the DOS domain, and the ELR3-6 domains of a Serrate protein.

14. The polypeptide of claim 1, wherein said Serrate protein is a mammalian Serrate protein.

15. The polypeptide of claim 1, wherein said Serrate protein is a human Serrate protein.

16. The polypeptide of claim 15, wherein said human Serrate protein is human Serrate-1.

17. The polypeptide of claim 15, where said human Serrate protein is human Serrate-2.

18. The polypeptide of claim 1, which is a purified polypeptide.

19. The polypeptide of claim 1 further comprising a non-Serrate protein amino acid sequence.

20. The polypeptide of claim 19, wherein said non-Serrate protein amino acid sequence is of an immunoglobulin Fc (IgFc) domain.

21. A derivative of the polypeptide as defined in claim 1, wherein 1-5 conservative amino acid substitutions are present in the Serrate protein sequence of said polypeptide.

22. A nucleic acid encoding the polypeptide of claim 1 or the derivative of claim 21.

23. (canceled)

24. A vector comprising a nucleotide sequence encoding the polypeptide of claim 1 operably linked to a promoter.

25. A host cell comprising the vector of claim 24.

26. A host cell expressing the polypeptide of claim 1.

27. (canceled)

28. (canceled)

29. (canceled)

30. A method of producing the polypeptide of claim 1, comprising culturing a host cell comprising a recombinant nucleic acid encoding the polypeptide operably linked to a promoter under conditions such that the polypeptide is produced by said host cell.

31. (canceled)

32. A pharmaceutical composition comprising a therapeutically effective amount of the polypeptide of claim 1 or the derivative of claim 21, and optionally further comprising a pharmaceutically acceptable carrier.

33. A kit comprising in one or more containers the pharmaceutical composition of claim 32.

34. A method for inhibiting Notch activity in a subject, comprising administering to said subject the polypeptide of claim 1 or the derivative of claim 21.

35. A method for inhibiting Notch activity in a subject, comprising administering to said subject a polypeptide comprising the DSL domain, the DOS domain, and the ELR4-6 domains of a Serrate protein.

36. (canceled)

37. (canceled)

38. A method for treating a disease involving increased Notch expression or activity relative to normal cells in a subject in need thereof, comprising administering to said subject the polypeptide of claim 1 or the derivative of claim 21.

39. A method for treating a disease involving increased Notch expression or activity relative to normal cells in a subject in need thereof, comprising administering to said subject a polypeptide comprising the DSL domain, the DOS domain, and the ELR4-6 domains of a Serrate protein, or a derivative of said polypeptide wherein 1-5 conservative amino acid substitutions are present in the Serrate protein sequence of said polypeptide.

40. (canceled)

41. (canceled)

42. (canceled)

43. An isolated antibody or fragment thereof that specifically binds an epitope containing at least a portion of ELR4-6 of a Serrate protein.

44. (canceled)

45. A method for increasing the activity of Notch in a subject, comprising administering to the subject the antibody or fragment thereof of claim 43.

46. A method for treating a disease involving decreased Notch expression or activity relative to normal cells in a subject in need thereof, comprising administering to said subject the antibody or fragment thereof of claim 43.

47. (canceled)
